(19) United States
(12) Patent Application Publication
(10) Pub. No.: US 2011/0142842 A1
(43) Pub. Date: Jun. 16, 2011

(54) POLYPEPTIDES

(75) Inventors: Johanna Petra Olweus, Oslo (NO); Fridtjof Lund-Johansen, Oslo (NO); Philippe Sebastien Walchli, Oslo (NO); Erlend Stronen, Oslo (NO); Jorunn Johansen, Oslo (NO)

(73) Assignee: Medinnova AS

(21) Appl. No.: 12/741,573

(22) PCT Filed: Nov. 5, 2008
(86) PCT No.: PCT/EP08/09605

§ 371 Date: Jan. 14, 2011

(30) Foreign Application Priority Data
Nov. 5, 2007 (GB) 0721686.4

(51) Int. Cl. A61K 39/395; A61K 31/7088; A61K 35/12; A61P 35/00

(52) U.S. Cl. 424/139.1; 514/44 R; 424/93.71

(57) ABSTRACT

There is disclosed a polypeptide consisting of between 7 and 100 amino acids and comprising: the sequence of the peptide of any one of SEQ. ID NOS: 1 to 145. There is also disclosed a T-cell receptor, or a peptide-binding fragment thereof, wherein the CDR3 region of the beta chain of the T-cell receptor comprises a glycine residue at position 5 from the N-terminus. The T-cell receptor is capable of binding a peptide consisting of the sequence of SEQ. ID NO. 18, when the peptide is presented on an HLA molecule of a first HLA allele.

### FIELD OF THE INVENTION

The present invention relates to polypeptides and to polynucleotides. The invention also relates to T-cells, T-cell receptors and polynucleotides encoding T-cell receptors and to methods of preparing T-cells. In addition, the present invention relates to methods of treating a patient suffering a disease caused by dysfunctional hematopoietic cells, in particular cancer, and kits for use in such a method.

### BACKGROUND ART

A patient suffering from a cancer such as leukemia will typically be treated with chemotherapy and the transplantation of bone marrow or peripheral blood rich in hematopoietic stem cells and harvested from a donor, called allogeneic stem cell transplantation (AST). The AST not only replaces the bone marrow stem cells destroyed by the chemotherapeutic treatment but also results in a so-called “graft-vs-leukemia” (GVL) effect. The GVL effect is thought to occur because the transplant contains allogeneic donor T-cells which are reactive with tumor cells in the patient (Horowitz M M, Gale R P, Sondel P M, et al. Graft-versus-leukemia reactions after AST. Blood. 1990; 75:555-562.).

The problem with such stem cell allografts is that in around 75% of transplanted patients a side effect known as “graft-versus-host disease” (GVHD) occurs (Appelbaum, F. R., Haematopoietic cell transplantation as immunotherapy. Nature, 2001. 411(6835): p. 385-9). GVHD can arise due to a mismatch in HLA alleles, or due to differences in polymorphic proteins, between the stem cell donor and the patient. It is to be appreciated that in a healthy individual, antigenic peptides complexed with MHC molecules are presented on the surface of antigen presenting cells to the T-cell receptors of T-cells. If the T-cell receptor is specific for the specific peptide-MHC complex, then the T-cell will interact with, and be stimulated by, the antigen presenting cell. However, the individual will normally have few T-cells circulating which are specific for self-peptides (such T-cells are destroyed in the thymus during the selection process), as such T-cells could give rise to an autoimmune response. That is to say, an individual will normally be tolerant to peptides from self-antigens.

In the case of a AST where the donor and the recipient have mismatched HLA alleles, the self-peptides of the recipient are presented to the T-cells on MHC molecules of a different HLA allele from those to which the T-cells were presented during the thymic selection process. The transplanted T-cells will recognize many of the self-peptides of the recipient as being foreign as they are in complex with an MHC molecule of a different allele. Such interactions may cause GVL as well as GVHD.

WO02/44207 discloses various HLA-binding peptides of human CD45 and the production of cytotoxic T lymphocytes (CTLs) that are specific for the peptides when complexed with MHC. It reports that such CTLs can be used to treat a patient with a hematopoietic malignancy. It also discloses the provision of allogeneic cells in the preparation of CTLs, that is to say the CTL are from an individual who is negative for a particular HLA and the peptide is presented by that particular HLA molecule by the antigen-presenting cell.

Chronic Lymphoid Leukaemia (CLL) is a disease that is still largely considered incurable. Many patients develop resistance to chemotherapy, frequently related to loss of p53. Monotherapy with anti-CD20 (CD20 being a B-lymphoid restricted antigen) is inefficient, possibly due to lowered susceptibility to antibody dependent cell-mediated cytotoxicity (ADCC) and complement-mediated cytotoxicity (CDC) (Bannerji, J Clin Oncol 2003). AHSCT is the only established treatment for CLL that can provide long-term disease control even for poor-risk patients.

The high treatment related mortality and morbidity mainly as a result of GVHD, hinders wide-spread use of allo-HSCT and has triggered efforts to develop strategies that allow autologous T cells to attack and kill CLL cells. Tumor-associated antigens have been identified in CLL cells with the ability of inducing antigen-specific T cells, including MDM2, a number of epitopes identified by SEREX technology, fibromodulin, survivin and p53 (Krackhardt Serex, Mayr Blood, Mayr Blood). Similarly, T cell lines have been generated by stimulation with autologous CLL cells (Krackhardt Blood, Buhmann Blood 99). None of the T cells generated in these studies were, however, demonstrated capable of killing CLL cells. It has thus proven difficult to generate therapeutically efficient CTL restricted by autologous HLA. Most TAA are overexpressed self-proteins. The results of these studies probably reflect that high-avidity T cells reactive with peptides from self-proteins presented by autologous HLA-molecules are deleted during thymic development.

Another study has demonstrated that priming with allogeneic, HLA-matched CLL cells readily allowed the induction of T cell lines from healthy donors that were capable of lysing primary CLL cells. However, there is an inherent risk of GVHD with this approach. The target minor histocompatibility antigens (mHAgs) were unidentified and will vary for each CTL line, and the mHAg expression on non-hematopoietic cell types is not known (Falkenburg).

The present invention seeks to provide T-cell receptors and polynucleotides encoding T-cell receptors. It also seeks to provide peptides from self-proteins other than CD45, and improved methods of producing cytotoxic T cells and other products.

### SUMMARY OF THE INVENTION

According to one aspect of the present invention, there is provided a T-cell receptor, or a peptide-binding fragment thereof, wherein the CDR3 region of the beta chain of the T-cell receptor comprises a glycine residue at position 5 from the N-terminus and wherein the T-cell receptor is capable of binding a peptide consisting of the sequence of SEQ. ID NO. 18, when the peptide is presented on an HLA molecule of a first HLA allele.

Conveniently, the C-terminal residues of the CDR3 region of the beta chain of the T-cell receptor are encoded by a J-2 gene segment, preferably wherein at least the two C-terminal residues of the CDR3 region of the beta chain of the T-cell receptor are encoded by a J-2 gene segment. It is particularly preferred that the two or three C-terminal residues of the CDR3 region of the beta chain are present, consecutively, within the five or six residues at the N-terminal of a J-2 gene segment.

Preferably, the CDR3 region of the beta chain of the T-cell receptor comprises amino acids encoded by J-2 gene segment J2-7, J2-5, J2-1, J2-2, J2-3 or J2-4.

Advantageously, the C-terminal residue of the CDR3 region of the beta chain of the T-cell receptor is an aromatic amino acid.

Conveniently, the aromatic amino acid is tyrosine or phenylalanine.

Preferably, the two amino acid residues at the C-terminus of the CDR3 region of the beta chain of the T-cell receptor consist of the sequence Gln Tyr.

Advantageously, the three amino acid residues at the C-terminus of the CDR3 region of the beta chain of the T-cell receptor consist of the sequence Glu Gln Tyr.

Conveniently, the CDR3 region of the beta chain of the T-cell receptor consists of between 9 and 12 amino acid residues, preferably 11 amino acid residues.

Preferably, the CDR3 region of the β chain of the T-cell receptor comprises the sequence of SEQ. ID NO. 167, 168 or 189.

Advantageously, the CDR3 region of the β chain of the T-cell receptor comprises the sequence of SEQ. ID NO. 169.

Conveniently, the CDR3 region of the β chain of the T-cell receptor comprises the sequence of any one of SEQ. ID NOS. 148 to 155 or 170 to 174.

Preferably, the T-cell receptor comprises an α chain. However, it is not essential to the invention that the T-cell receptor or fragment thereof comprises an α chain.

Advantageously, the α chain in combination with a β chain is capable of binding a peptide of the CD20 protein when the peptide is presented on an HLA molecule of a first HLA allele.

Conveniently, wherein the peptide of the CD20 protein consists of the sequence of SEQ. ID NO. 18.

Preferably, the CDR3 region of the α chain of the T-cell receptor comprises the sequence of any one of SEQ. ID NOS. 175 to 188 or 190.

According to another aspect of the present invention there is provided a polynucleotide comprising a sequence encoding the T-cell receptor, or peptide-binding fragment thereof, of the invention.

According to a further aspect of the present invention there is provided a T-cell receptor, or a peptide-binding fragment thereof, for use in a method of treating cancer or a disease related to a dysfunction in hematopoietic cells, wherein the T-cell receptor is capable of binding a polypeptide consisting of the sequence of the peptide of SEQ ID NO: 18 when the peptide is presented on an HLA molecule of a first HLA allele, and wherein the T-cell receptor is obtainable from an individual who is HLA negative for said first HLA allele.

Conveniently, the T-cell receptor is a T-cell receptor of the invention.

According to another aspect of the present invention there is provided a polynucleotide for use in a method of treating cancer or a disease related to a dysfunction in hematopoietic cells, wherein the polynucleotide comprises a sequence encoding a T-cell receptor capable of binding a polypeptide consisting of the sequence of the peptide of SEQ ID NO: 18 when the peptide is presented on an HLA molecule of a first HLA allele, and wherein the T-cell receptor is obtainable from an individual who is HLA negative for said first HLA allele.

Preferably, the polynucleotide is a polynucleotide of the invention.

According to one aspect of the present invention there is provided a polypeptide consisting of between 7 and 100 amino acids and comprising: the sequence of the peptide of any one of SEQ ID NOS: 1 to 145; or a sequence with at least a specific level of identity to the peptide of any one of SEQ ID NOS: 1 to 145, wherein the specific level of identity is 60%.

Conveniently, the polypeptide consists of at least 8 or 9 amino acids.

Preferably, the polypeptide consists of less than 80, 70, 60, 50, 40, 30, 25, 20 or 15 amino acids.

Advantageously, the polypeptide consists of the sequence of the peptide of any one of SEQ ID NOS: 1 to 145 or a sequence with at least the specific level of identity to the peptide of any one of SEQ ID NOS: 1 to 145.

Conveniently, the specific level of identity is 70%, 80%, 90%, 95% or 99%.

Preferably, the polypeptide is naturally displayed by MHC molecules on the target cells.

Advantageously, the polypeptide is naturally displayed by MHC molecules on the target cells, as demonstrated by a cytotoxicity assay and/or degranulation assay using a CTL line specific for the polypeptide in complex with HLA-A*0201, preferably wherein the target cells are hematopoietic, more preferably peripheral blood leukocytes or a leukemia or lymphoma cell line.

Conveniently, the polypeptide comprises the sequence of any one of SEQ ID NOS: 1 to 48, 50 to 55, 58 to 66, 68, 70 to 84, 86 to 89, 91 to 100, 102 to 106, 108 to 117, 119 to 124 or 126 to 145 or a sequence with at least the specific level of identity thereto.

According to another aspect of the present invention there is provided a polynucleotide consisting of fewer than 300 nucleotides and encoding a polypeptide of the invention.

According to a further aspect of the present invention, there is provided a method of preparing T-cells suitable for delivery to a patient suffering from cancer, comprising the steps of:
(i) providing dendritic cells expressing an HLA molecule of a first HLA allele;
(ii) locating a peptide in the binding groove of the HLA molecule; and
(iii) priming T-cells with the dendritic cells, wherein the T-cells are obtained from an individual who is HLA negative for said first HLA allele.

Preferably, step (i) comprises providing a dendritic cell that is negative for the first HLA allele and transfecting the dendritic cell with a polynucleotide encoding an HLA molecule of said first HLA allele.

Advantageously, the T-cells are obtained from the same individual as the dendritic cells.

Alternatively, the dendritic cells are obtained from an individual having a single HLA mismatch with the individual from whom the T-cells are obtained.

Conveniently, in step (i), the polynucleotide encoding an HLA molecule of said first HLA allele is mRNA.

Alternatively, step (i) the polynucleotide encoding an HLA molecule of said first HLA allele is DNA.

Preferably, the first HLA allele is an HLA class I allele.

Advantageously, the first HLA allele is HLA-A*0201.

Conveniently, the method further comprises the step of:
(iv) enriching the primed T-cells by binding the T-cells to structures comprising HLA molecules of the first HLA allele presenting the peptide.

Preferably, the structures comprise multimers of recombinant HLA molecules, the HLA molecules having the peptide located in their binding grooves.

Advantageously, the multimers are tetramers or pentamers.

Conveniently, step (iv) further comprises the step of isolating the cells binding to the structures by binding the structures to magnetic beads or by flow cytometric cell sorting.

Preferably, step (ii) comprises loading the peptide in the binding groove of the HLA molecule.

Advantageously, step (ii) comprises transfecting the dendritic cells with a nucleic acid encoding a fusion protein comprising the peptide and the HLA molecule.

Conveniently, the peptide is a cell type specific peptide.

Preferably, the peptide is a polypeptide of the invention.

According to another aspect of the present invention, there is provided a T-cell receptor capable of binding a polypeptide of the invention when the polypeptide is presented on an HLA molecule of a first HLA allele.

Advantageously, the T-cell receptor is obtainable from an individual who is HLA negative for said first allele.

According to a further aspect of the present invention, there is provided a T-cell displaying a T-cell receptor of the invention.

According to another aspect of the present invention, there is provided a polynucleotide encoding a T-cell receptor of the invention.

According to a further aspect of the present invention, there is provided a method of treating a patient suffering from cancer or a disease related to a dysfunction in hematopoietic cells comprising the steps of:
(i) providing a T-cell of the invention or a T-cell prepared in accordance with the methods of the invention; and
(ii) administering the T-cells to the patient.

Preferably, step (i) comprises the step of transfecting a cell with a polynucleotide of the invention and expressing the polynucleotide in order to provide the T-cell.

According to another aspect of the present invention, there is provided the use of a T-cell of the invention, a polynucleotide of the invention or a T-cell prepared in accordance with the methods of the invention for the manufacture of a medicament for the treatment of cancer or a disease related to a dysfunction in hematopoietic cells.

Conveniently, the patient is HLA positive for the first HLA allele.

Preferably, step (i) comprises providing a plurality of sets of T-cells as defined, each set of T-cells being capable of binding a different peptide.

Advantageously, the T-cells comprise a plurality of sets of T-cells, each set of T-cells being capable of binding a different peptide.

According to a further aspect of the present invention, there is provided a method of treating a patient suffering from cancer or a disease related to a dysfunction in hematopoietic cells, the patient being HLA positive for a first HLA allele, the method comprising the steps of:
(i) providing a first T-cell displaying a first T-cell receptor capable of binding a first peptide, present in the patient, when presented by an HLA molecule of the first allele, the first T-cell receptor being obtainable from an individual who is HLA negative for the first HLA allele;
(ii) providing a second T-cell which displays a second T-cell receptor capable of binding a second peptide, present in the patient, when presented by an HLA molecule of the first HLA allele, the second T-cell receptor being obtainable from an individual who is HLA negative for the first HLA allele; and
(iii) administering the first and second T-cells simultaneously or sequentially.

Conveniently, said first and second T-cells are the same, the first and second T-cell receptors being displayed on the same cells.

Preferably, the first HLA allele is an HLA class I allele.

Advantageously, the first HLA allele is HLA-A*0201.

Conveniently, the method further comprises providing at least a third T-cell which displays a third T-cell receptor, the third T-cell receptor being capable of binding a third peptide, present in the patient, when presented by an HLA molecule of the first HLA allele, the third T-cell receptor being obtainable from an individual who is HLA negative for the first HLA allele

According to another aspect of the present invention, there is provided a kit suitable for treating a patient suffering from cancer or a disease related to a dysfunction in hematopoietic cells the patient being HLA positive for a first HLA allele, the kit comprising:
(i) a first T-cell receptor according to the invention; and
(ii) a second T-cell receptor according to the invention wherein the first T-cell receptor is capable of binding a different peptide from the second T-cell receptor.

Conveniently, the kit further comprises at least a third T-cell receptor according to the invention wherein the third T-cell receptor is capable of binding a different peptide from the first and second T-cell receptors.

Preferably, each of said first, second and optionally third T-cell receptors are displayed by first, second and optionally third T-cells, respectively, or by a single T cell population.

According to a further aspect of the present invention, there is provided the use of a kit of the invention for the manufacture of a medicament for the treatment of cancer or a disease related to a dysfunction in hematopoietic cells.

Preferably, the cancer is a cancer of the hematopoietic cells, more preferably leukemia, lymphoma or multiple myeloma.

Alternatively, the disease related to a dysfunction in hematopoietic cells is an autoimmune disease, preferably multiple sclerosis, rheumatoid arthritis, systemic lupus erythematosus, immune thrombocytopenic purpura, chronic cold agglutinin disease, IgM-mediated neuropathies or mixed cryoglobulinemia, or an inherited immunodeficiency, or bone marrow failure, preferably aplastic anemia or paroxysmal nocturnal hemoglobinuria.

According to another aspect of the present invention there is provided a polynucleotide encoding a fusion protein, the fusion protein comprising a polypeptide of the invention and an HLA class I alpha chain molecule.

In this specification, where a peptide is said to bind an MHC molecule that is to say the peptide is bound with a particular binding affinity of, for example, less than 1 μM.

In this specification, a “peptide-binding fragment” of a T-cell receptor is any fragment (including non-contiguous fragments) of a T-cell receptor which binds the same peptide as the complete receptor. Such fragments comprise the same CDR3 region of at least one chain of the T-cell receptor and typically also comprise the same CDR1 and 2 regions. The fragment may be all or part of a T-cell receptor α chain or β chain.

In this specification, where a T-cell receptor is described as “obtainable” from an individual this means that at least the minimum defining components of the T-cell receptor (e.g. the CDR3 region of the β chain) are of a sequence that is present in the T-cell receptors of T-cells that are naturally produced by the individual.

In this specification, the following abbreviations are used.

“AHSCT” means “Allogeneic Hematopoietic Stem Cell Transplantation”.

“AST” means “Allogeneic Stem Cell Transplantation”.

“GVL” means “Graft-vs-Leukemia”.

“GVHD” means “Graft-Versus-Host Disease”.

“moDC” means “Monocyte-Derived Dendritic Cells”.

“TAA” means “Tumor-Associated Antigen”.

### FIGURES

FIG. 1A is a graph showing expression of HLA-A*0201 of moDC transfected before or after maturation.

FIG. 1B is a graph showing expression of HLA-A*0201 of moDC transfected with varying concentrations of mRNA.

FIG. 1C is a graph showing expression of HLA-A*0201 at time points after transfection. Tests using two different concentrations of mRNA were carried out.

FIG. 1D is a panel of four graphs showing expression of the maturation markers HLA-DR, CD80, CD83 and CD86 of moDC transfected with HLA-A*0201.

FIG. 2A is a graph showing cell viability of moDC 24 or 48 hours after transfection with HLA-A*0201 mRNA with varying pulse lengths, relative to non-transfected cells.

FIG. 2B is a graph showing expression of HLA-A*0201 by moDC 24 or 48 hours after transfection with HLA-A*0201 mRNA with varying pulse lengths.

FIG. 3A is a graph showing relative proportions of pentamer positive and CD8⁺ T cells from two HLA-A*0201-negative donors co-cultured with autologous moDC transfected with HLA-A*0201 and pulsed with peptide.

FIG. 3B is a graph showing relative proportions of pentamer positive, CD8⁺ T cells following sequential purification steps.

FIG. 3C is a graph showing the expansion of pentamer positive cells against time.

FIG. 4A is a graph showing relative proportions of CD8⁺ T cells positive for IFN-gamma and/or CD107a/b following stimulation with HLA-A*0201 positive and negative cells, with and without relevant peptide.

FIG. 4B is bar graph showing production of IFN-gamma and/or degranulation in CD8⁺ T cells in response to stimulation with autologous EBV-transformed lymphoblastoid cell lines (EBV-LCL) with and without HLA-A*0201-transfection and pulsed with relevant or non-relevant peptide.

FIG. 4C is a graph showing the proportion of T2-cells lysed by pentamer positive cells after loading of the T2 cells, at varying concentrations, with relevant (MART-1) or non-relevant (CMV) peptide.

FIGS. 5A, 5B and 5C are graphs showing CTL responses to EBV-LCL or tumor cell lines that are positive or negative for HLA-A*0201 and positive or negative for MART-1 expression with and without pulsing with MART-1 or CMV-peptide (control) and/or blocking of MHC class I prior to stimulation.

FIGS. 6A, 6B, 6C, 6D and 6E are graphs showing CTL responses to EBV-LCL or tumor cell lines that are positive or negative for HLA-A*0201 and positive or negative for CD20 expression with and without pulsing with CD20 peptide (#19 in Table 1, referred to as pep 19) and/or blocking of MHC class I prior to stimulation.

FIG. 7 shows graphs demonstrating the optimization of mRNA transfection of moDCs with HLA-A*0201. A. MoDCs from an HLA-A*0201⁻ individual were electoporated (1.25 kV/cm for 3 ms) in the presence (black histogram) or absence of HLA-A*0201 mRNA, cultured for 18 h and labelled with anti-HLA-A*0201 antibody. B moDCs were transfected before (immature) or after maturation with LPS and TNF-α. Relative mean fluorescence intensity (MFI) was calculated by dividing the MFI of the cells electroporated with mRNA with the MFI of cells electroporated without mRNA. Grey lines; individual experiments, black line; mean of 4 experiments. C Mature MoDCs were electroporated for the indicated time in the presence of HLA-A*0201 mRNA, and the expression of HLA-A*0201 (left diagram) and viability (right diagram) was analyzed by flow cytometry at 24 (white bars) and 48 (black bars) hours post-transfection. Graphs display mean+SD, and * indicates p<0.05, n=4. D HLA-A*0201 expression on moDC, analyzed at 4, 12 and 24 hours after transfection. Grey lines; individual experiments, black line; mean of 4 experiments.

FIG. 8 relates to the induction of MART-1 positive CTL from HLA-A2 negative donors A PBMC from HLA-A*0201⁻ donors were stained with anti-CD3, CD8 and A2/MART-1 pentamer immediately after isolation (left panels) or after 12 days of co-culture with MART-1 peptide-pulsed A2-moDC (right panels). Cultured cells were also labeled with a control pentamer (HLA-A*0201-CMVpp65₄₉₅₋₅₀₃) (middle panels). A total of 100,000-300,000 CD3⁺CD8⁺ events were analyzed by flow cytometry, and all acquired events are displayed. The numbers refer to the frequency of pentamer⁺ cells in the CD3⁺CD8⁺ population. Each row represents one out of 11 separate donors. B A2/MART-1 pentamer⁺ cells sorted on day 12 were reanalyzed immediately after sorting (left panel). Sorted cells were expanded for 4 weeks and stained with control pentamer (middle panel) and MART-1 pentamer (right panel). One cell line representative of 5 is shown. C Sorted, pentamer⁺ cells were expanded in the presence of PHA, IL-2, and IL-15 on irradiated, allogeneic PBMCs. Absolute numbers are extrapolated from a starting population of 0.5×10⁶ cells (right y-axis, filled circles). The data are representative of five experiments. D A2/MART-1 pentamer⁺ cell lines were labeled with indicated antibodies to TCR Vβ chains. The bars indicate percentage of positive cells in each of four T cell lines derived from four separate donors.

FIG. 9 relates to allo-restricted, MART-1 specific T cell lines produce IFN-γ and mobilize CD107a/b upon antigen-specific stimulation. A and B A2/MART-1 pentamer⁺ T cell lines were co-cultured for five hours with autologous EBV-LCL transfected with HLA-A*0201, loaded with peptide (MART-1 or control) and washed, at an E:T ratio of 2:1. Production of IFN-γ and mobilization of CD107a/b was measured by flow cytometry. In A the results from one representative experiment is shown, whereas B displays data from T cell lines generated from four different donors. Responses to EBV-LCL pulsed with different peptide concentrations were significantly higher than those to HLA-A*0201-transfected EBV-LCL loaded with control peptide (*;p<0.05) Error bars represent SD of four experiments. C Lysis of T2 cells loaded with MART-1 peptide or control peptide and washed before co-culture with an A2/MART-1 pentamer⁺ T cell line in a standard 4-hour ⁵¹Cr-release assay at an E:T ratio of 10:1. Data from one representative of 2 experiments are shown.

FIG. 10 relates to allo-restricted, MART-1 specific T cells kill HLA-A*0201 positive melanoma cells. A MART-1 specific, HLA-A*0201-restricted T cell lines were stimulated for 5 hours with autologous EBV-LCL or the melanoma tumor cell lines FM-57, Malme3M and Mel202 at an E:T ratio of 2:1, and the production of IFN-γ and degranulation (mobilization of CD107a/b) was measured by flow cytometry.

Summarized data from 4 individual donors and experiments are shown (mean+SD). B MART-1/HLA-A*0201-restricted T cell lines were stimulated for 5 hours with allogeneic EBV-LCL or melanoma cells at an E:T ratio of 2:1. Percent specific lysis was calculated by flow cytometric counting of the absolute number of live cells and comparing this to the number of live cells in control plates where no effector cells had been added (for details see materials and methods of Example 9). Data from 1 representative of 3 individual donors and 4 experiments is shown. In A and B target cells are transfected with HLA-A*0201, loaded with 10 μM peptide and washed, and blocked by anti-HLA class I antibodies as indicated.

FIG. 11 relates to allo-restricted, CD33₉₋₁₇ peptide specific T cells produce IFN-γ and degranulate in response to peptide-pulsed, HLA-A*0201 positive targets. A PBMCs from HLA-A*0201⁻ donors were stained with anti-CD3, CD8 and A2/CD33 pentamer or control pentamer following 12 days of co-culture with CD33₉₋₁₇ peptide-pulsed A2-moDC, and re-stimulation with peptide-pulsed A2-EBV-LCL for an additional week (upper two panels). Numbers refer to the frequency of pentamer⁺ cells in the CD3⁺CD8⁺ population. One of three representative donors is shown. Sorted, A2/CD33 pentamer⁺ cells were expanded for 19 days in the presence of PHA, IL-2, and IL-15 on allogeneic PBMC, and stained with anti-CD3, CD8 and A2/CD33 pentamer or control pentamer (lower two panels). B An A2/CD33 pentamer⁺ T cell line was co-cultured for five hours with autologous EBV-LCL transfected with HLA-A*0201, loaded with 10 μM peptide (CD33₉₋₁₇ or control) and washed, at an E:T ratio of 2:1. Numbers represent the frequencies of cells producing IFN-γ and/or mobilizing CD107a/b, as measured by flow cytometry.

FIG. 12 relates to the induction of CTLs specific for CD20 and HLA-A*0201 by dendritic cells engineered to express allo-HLA-peptide complexes. PBMC from 6 different HLA-A*0201 negative donors and autologous HLA-A*0201-transfected moDCs pulsed with CD20 peptide, were co-cultured for 19 days (A), or PBMC from 2 HLA-A*0201 positive donors and autologous moDC pulsed with CD20 peptide were co-cultured for 33 days (B), harvested and stained with mAbs to CD3 and CD8 and A2/CD20 pentamer or control pentamer (HLA-A*0201-CMVpp65₄₉₅₋₅₀₃). All dot plots are gated on CD3⁺ cells, and PI positive cells are excluded. Numbers represent the frequency of pentamer⁺ cells in the CD3⁺CD8⁺population.

FIG. 13 relates to the efficient expansion of A2/CD20 pentamer positive T cell lines with an early effector memory phenotype. A A2/CD20 pentamer positive cells sorted on day 19 were reanalyzed immediately after sorting (left). Sorted cells were expanded for 5 weeks and stained with A2/CD20 pentamer or control pentamer. One cell line representative of 5 is shown. B Pentamer positive cells were expanded in the presence of PHA, IL-2, IL-15 and irradiated allogeneic PBMCs. Cells from 5 donors expanded well, whereas cells from the remaining donor failed to expand. Absolute numbers are extrapolated from the starting population and weekly fold-expansion. C T cell lines were immuno-phenotyped following 6 weeks of culture. Plots show results from one representative donor on day 41 and are representative of the 3 cell lines tested. Black and gray lines represents staining with the indicated antibodies and isotype control, respectively, and numbers represent percentage positive cells.

FIG. 14 relates to allo-restricted CD20-specific T cells which produce interferon-γ and mobilize CD107a/b upon antigen-specific stimulation. A and B A2/CD20 pentamer⁺ T cell lines were co-cultured for five hours with EBV-LCL or leukemia cell lines JVM-2 or THP-1 that were transfected or not with HLA-A*0201 and/or loaded with peptide where indicated. E:T ratio was 2:1 and anti-class I antibodies were added as indicated. Production of IFN-γ and mobilization of CD107a/b was measured by flow cytometry. A and B show results from two separate T cell lines and are representative of 4 T cell lines and 7 experiments. In B data are shown as mean and standard deviation (SD) of triplicates.

FIG. 15 relates to allo-restricted CD20-specific T cells which kill HLA-A*0201 positive CD20 positive target cells. CD20-specific, HLA-A*0201-restricted T cell lines were co-cultured with EBV-LCL or leukemia cell lines for 5 hours at an E:T ratio of 2:1. Percent specific lysis was calculated by flow cytometric counting of the reduction in absolute counts of live target cells compared to the number of live cells in control plates without effector cells. Results are shown as mean and SD of triplicates using one CTL line representative of 4 T cell lines and 5 experiments.

FIG. 16 relates to allo-restricted CD20-specific T cells which efficiently kill primary CLL cells. A CD20-specific, HLA-A*0201-restricted CTL lines were co-cultured with

CLL cells from four HLA-A*0201 positive or 2 HLA-A*0201 negative patients or CD4+ T cells from healthy HLA-A*0201 positive donors, at an E:T ratio of 1:1 for 5 hours. Specific lysis was measured as the reduction in percentage of live cells compared to the percentage of live cells in control plates without effector cells. Data show mean values for specific lysis in 4 separate experiments and 3 different CTL lines, error bars indicate SD, stars indicate significance relative to lysis of CD4+ T cells (**p<0.01, ***p<0.001) B Histograms gated on CD19+CD5+ CLL cells from 6 patients showing expression of HLA-A*0201, CD20, CD80 and CD86, respectively.

FIG. 17 is the sequence of an exemplary TCR α chain where CDRs 1, 2 and 3 are shown together with the gene segments from the chain is formed.

FIG. 18 is the sequence of an exemplary TCR β chain where CDRs 1, 2 and 3 are shown together with the gene segments from the chain is formed.

FIG. 19 shows the alignment of the sequences of TRBC 1 and 2.

### DETAILED DESCRIPTION

In embodiments of the present invention there are provided polypeptides consisting of between 7 and 100 amino acids and comprising one of SEQ ID NOS: 1 to 145. The peptides are derived from proteins specific for hematopoietic cells. In preferred embodiments, “specific for” means that the proteins exist in concentrations at least 10 times higher than the mean concentration in other cell types, such as epithelial cells, endothelial cells and/or hepatocytes.

SEQ ID NOS: 1 to 48, 50 to 55, 58 to 66, 68, 70 to 84, 86 to 89, 91 to 100, 102 to 106, 108 to 117, 119 to 124 or 126 to 145 are particularly preferred since these peptides have MHC binding assay scores higher than a control peptide (see Table 1). However, it is to be understood that in other embodiments a different control peptide may be chosen (e.g. a MART-1 peptide) and preferred peptides may be selected with reference to the MHC binding assay scores relative to that control peptide.

In some alternative embodiments, the peptides do not correspond precisely to those set out in SEQ ID NOS: 1 to 145 but are instead proteins with at least 60% identity thereto, or preferably, 70%, 80%, 90%, 95% or 99% identity.

In this specification, the percentage “identity” between two sequences is determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

Thus in some embodiments, one or more amino acids of the peptides are omitted or are substituted for a similar amino acid. A similar amino acid is one which has a side chain moiety with related properties and the naturally occurring amino acids may be categorized into the following group. The group having basic side chains: lysine, arginine, histidine. The group having acidic side chains: aspartic acid and glutamic acid. The group having uncharged polar side chains: asparagine, glutamine, serine, threonine and tyrosine. The group having non-polar side chains: glycine, alanine, valine, leucine, isoleucine, proline, phenylalanine, methionine, tryptophan and cysteine. Therefore it is preferred to substitute amino acids within these groups.

The peptides are capable of binding MHC molecules and therefore it is also preferred that any amino acid substitutions maintain or improve the capacity to bind MHC molecules. For example, if the peptide is capable of binding MHC molecules of the HLA-A*0201 allele then it is preferred that the amino acids at position 2 of the peptide (i.e. the second amino acid from the N-terminus) are leucine or methionine, although isoleucine, valine, alanine and threonine are also tolerated. It is also preferred that the amino acid at position 9 or 10 is valine, leucine or isoleucine, although alanine, methionine and threonine are also tolerated. The preferred MHC binding motifs or other HLA alleles are disclosed in Celis et al., Molecular Immunology, Vol. 31, 8, Dec. 1994, pages 1423 to 1430.

It is generally preferred that the polypeptide conforms with the chemistry of naturally occurring polypeptides (although it may be synthesized in vitro) but in some alternative embodiments the polypeptide is a peptidomimetic, that is to say a modification of a polypeptide in a manner that will not naturally occur. Such peptidomimetics include the replacement of naturally occurring amino acids with synthetic amino acids and/or a modification of the polypeptide backbone. For example in some embodiments, the peptide bonds are replaced with a reverse peptide bond to generate a retro-inverso peptidomimetic (see Méziére et al J Immunol. 1997 Oct. 1; 159(7):3230-7, which is incorporated herein by reference.) Alternatively, the amino acids are linked by a covalent bond other than a peptide bond but which maintains the spacing and orientation of the amino acid residues forming the polymer chain.

It is to be appreciated that in some embodiments, the polypeptide comprises additional amino acid sequences aside from those of one of the specific peptides sequence of SEQ ID NOS: 1 to 145. These additional amino acid sequences may comprise binding motifs or translocation signal peptides or may include protease target sequences to enable a polypeptide to be cleaved in order to release the specific peptide.

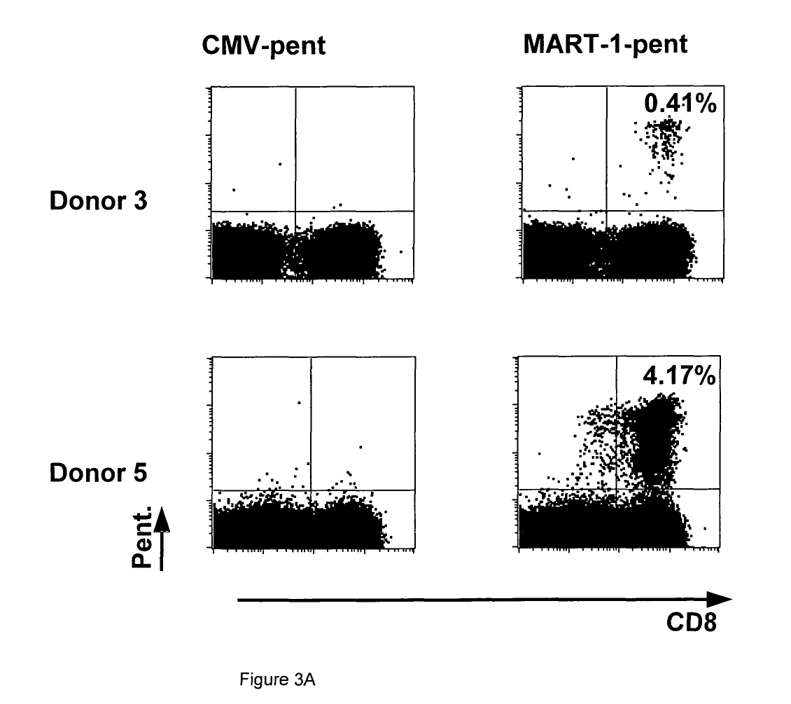

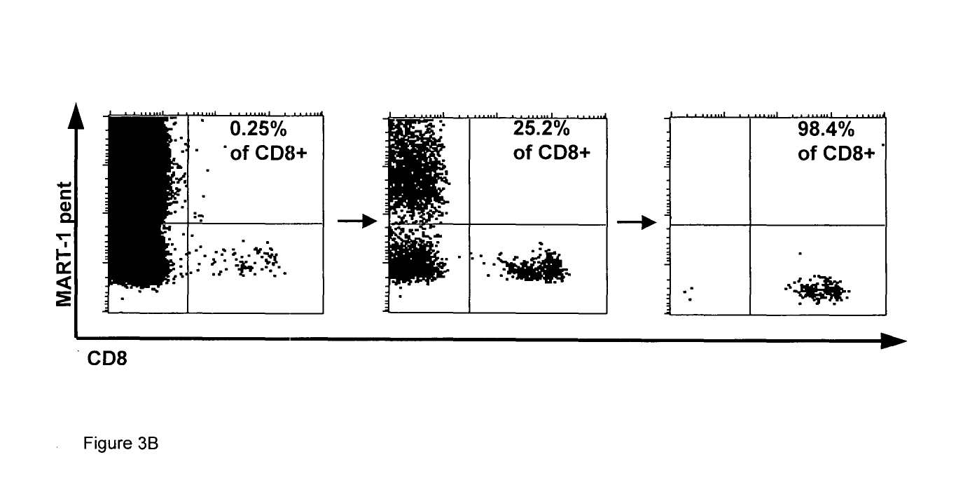

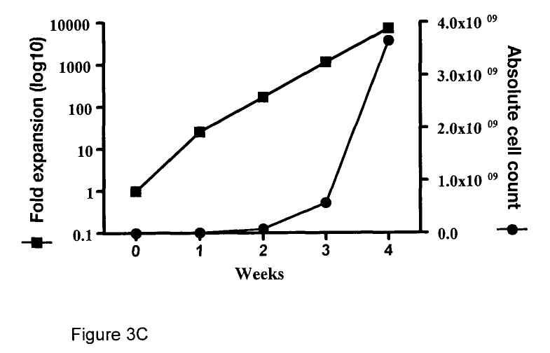

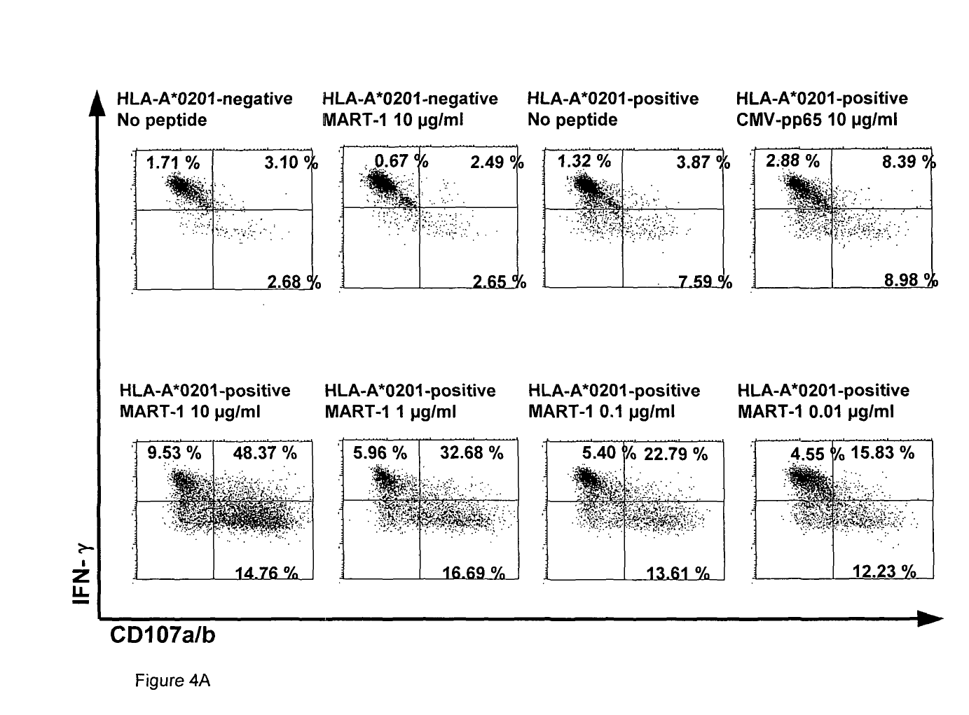

A stepwise scheme was followed to identify peptides that qualify as targets for cancer therapy. The peptides of SEQ ID NOS: 1 to 145 was generated as follows. Candidate proteins were chosen according to specificity for hematopoietic cells. Peptide sequences from the candidate protein sequences that were predicted to bind to HLA-A*0201 were identified by a computer algorithm available at http://www.cbs.dtu.dk/services/NetMHC/. Peptides were selected for synthesis according to predicted affinity with a cut-off value of 500 nM or less, but also higher values may be chosen. Peptides were synthesized and binding to HLA-A*0201 was biochemically confirmed. Peptide binding was compared with the binding achieved with a pass/fail control peptide, designated 100%, and with a positive control peptide, as shown in Table I. Corresponding HLA-A*0201-peptide pentamers were synthesized for peptides with a binding affinity above the pass/fail control peptide. These peptides were tested for ability to generate a T cell line specifically reacting with the specific peptide-HLA-A*0201 complex, detected as pentamer positive T cells (shown in Example 3 for the MART-1 peptide, and in Table 2 as percentage pentamer positive cells). Pentamer positive cells detected already on day 12 or day 19 indicate a high immunogenicity for the corresponding peptide. Pentamer positive cells were further enriched by magnetic bead or flow cytometric cell sorting (FIG. 3B) and expanded in culture (FIG. 3C). Further functional testing of the cell lines revealed which of the peptides are naturally expressed by the target cells, as shown in Examples 5 and 6. Example 5 shows that T cell lines generated against the modified MART-1 peptide recognize and respond to tumor cell lines naturally expressing MART-1 in combination with induced or naturally expressed HLA-A*0201. Example 6 shows that T cell lines generated against peptide 19, derived from CD20, respond to target cells naturally expressing CD20 in combination with induced or naturally expressed HLA-A*0201. The responses measured include production of the cytokine interferon-gamma, degranulation and killing of target cells.

In some embodiments, the polypeptide of the invention is administered directly to a patient as a vaccine. Thus the peptides of the invention are immunogenic epitopes of hematopoietic cell specific proteins and are used in order to elicit a T-cell response to their respective proteins. In some embodiments, the polypeptide of the invention is administered directly to a patient as a vaccine. For example, in a patient that has leukemia, a polypeptide comprising a peptide from a hematopoietic cell specific protein is administered to the patient in order to elicit a T-cell response to the protein. The T-cell response leads to death of hematopoietic cells, including the cancerous cells, but is specific to these cells and does not result in an immune response to other cell types. The patient may also be administered chemotherapy and/or radiotherapy in order to eliminate cancerous cells. In view of the absence of operative hematopoietic cells, the patient receives a stem cell transplantation following the treatment.

It is also to be noted that, in many patients, directly administering such a polypeptide will not elicit a T-cell response because the cell specific protein is a “self protein” and any T-cells that are capable of binding the polypeptide when presented on an MHC molecule of the HLA alleles of the patient are tolerised. That is to say T-cells that would be reactive are either destroyed in the thymus of the patient during the selection process or are inactivated through central or peripheral tolerance mechanisms. Therefore, it is preferred that the polypeptides of the invention are used to generate T-cells obtained, or obtainable, from an allogeneic donor individual (e.g. a human donor or a humanized mouse). This individual should preferably be HLA negative for an HLA allele of which the patient is HLA positive. For example, if the patient is HLA positive for the HLA allele HLA-A*0201 then T-cells are obtained from an individual who is negative for HLA-A*0201. It is generally preferred that the donor individual is otherwise HLA-identical to the patient. Antigen presenting cells (APCs) are then provided which display MHC molecules of the HLA-A*0201 allele and which are loaded with the peptide. The T-cells of the donor individual are then primed with the APCs and the resulting cells are allowed to proliferate.

The proliferated CD8⁺ T-cells which are capable of binding the peptide of the invention when in complex with the HLA-A*0201 antigen are then enriched using artificial structures which comprise a plurality of peptide-MHC molecules (e.g. pentamers or tetramers). The CD8⁺ T cells specific for the particular peptide-HLA-A*0201 complex within the mixture of T cells have the capacity to bind to these structures when mixed with them. The T cells are subsequently mixed with magnetic beads with the capacity to bind the artificial structures. The artificial structures and the T cells bound to them are then removed from the remainder of the mixture by magnetic attraction of the beads.

As an alternative to magnetic sorting of the artificial structures, flow cytometric cell sorting may be used. Further details of the artificial structures are provided in EP1670823, EP1678206, WO2006/082387 and WO2004/018520 each of which is incorporated herein by reference.

The selected T-cells are then expanded in culture into the numbers necessary for functional testing. This testing reveals whether the immunogenic peptide used for generating the T cells is actually expressed in the target cells, such as leukemia or lymphoma cells. If the peptide is expressed, the generated T cells are able to kill all cells expressing the target peptide/protein, whereas killing of other cells types does not occur or is very limited. Examples 5 and 6 show that the T cells generated against the MART-1 peptide and the CD20 peptide (peptide #19) kill target cells naturally expressing the protein and HLA-A*0201, but not target cells expressing only protein or HLA-A*0201 or none of them. This selection allows for the determination of which immunogenic peptides are expressed in vivo and thus therapeutic targets.

It is to be appreciated that, because the APCs present the peptide on an MHC molecule of an HLA allele to which the T-cells were not exposed in the donor individual, there are T-cells in the repertoire of the donor individual which are capable of binding the peptide-MHC molecule complex even though the peptide is from a normal self protein. Thus the T-cells are administered to the patient and elicit an immune response in view of the HLA mismatch between the donor individual and the patient.

In one embodiment, in which the patient has leukemia, the peptide is an epitope of a protein specific for hematopoietic cells. T-cells are generated as described above and are administered to the patient as part of an AST. The stem cells may be obtained from the same donor individual from whom the T-cells are obtained or may be obtained from a different donor. The T-cells react with the hematopoietic cells of the patient and eliminate the hematopoietic cells, including the cancerous cells, but these are replaced by the healthy hematopoietic stem cells in the donated transplant.

Thus the advantage of the polypeptides of the present invention is that they need not be cancer-specific polypeptides which have proven very difficult to identify. T-cells are instead generated against a particular cell type specific protein whether or not it is cancer associated. Furthermore, because T-cells are specific for epitopes of that protein, a non-specific graft-versus-host-disease is limited or does not arise.

While the length of the polypeptide may vary between 7 and 100 amino acids long, it is preferred that it is 8 or 9 amino acids long as this is the optimum length for binding to MHC Class I molecules. However, it is to be understood that longer polypeptides may be used since proteins are naturally subjected to proteolytic degradation as part of antigen presentation on antigen presenting cells and therefore longer polypeptides sequences will generally be cleaved into peptides of around 8 or 9 amino acid residues.

Polypeptides of the invention may be synthesised by the Fmoc-polyamide mode of solid-phase peptide synthesis as disclosed by Lu et al (1981) J. Org. Chem. 46, 3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethylacrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesied. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers. Reagents for peptide synthesis are generally available from Calbiochem-Novabiochem (UK) Ltd, Nottingham, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

In other embodiments of the present invention, a polynucleotide is provided which encodes a polypeptide of the invention. The polynucleotides are generally less than 300 nucleotides in length. In addition to the sequence specifically encoding the polypeptide of the invention, the polynucleotide may contain other sequences such as primer sites, transcription factor binding sites, vector insertion sites and sequences which resist nucleolytic degradation (e.g. polyadenosine tails). The polynucleotides may be DNA or RNA and may include synthetic nucleotides, provided that the polynucleotide is still capable of being translated in order to synthesize a polypeptide of the invention.

Such polynucleotides are used in order to synthesize the polypeptides of the invention. In some embodiments, this is done by in vitro translation but in other embodiments, the polynucleotide is inserted into a vector (e.g. a plasmid) and expressed in a host cell such as E coli. In still further embodiments, the polynucleotide is transfected into antigen presenting cells in order to be expressed therein. In some embodiments, the antigen presenting cell is a dendritic cell which is used to prepare T-cells as explained above. In other embodiments, the antigen presenting cell is within a patient and thus the polynucleotide is administered as part of a gene therapy.

In the above described embodiments, a peptide of the invention is located within the MHC molecule of a dendritic cell in order to generate suitable T-cells. In some embodiments, the MHC molecule is loaded with the peptide extracellularly by incubating cells at 37° C., 5% CO₂ for 4 hours with varying concentrations of peptide as indicated in the examples, then washed once in serum-free RPMI. However, in alternative embodiments, the antigen presenting cells are transfected with a polynucleotide encoding a fusion protein comprising the peptide connected to at least an MHC Class I molecule alpha chain by a flexible linker peptide. Thus, when expressed, the fusion protein results in the peptide occupying the MHC Class I binding groove. Suitable MHC Class I molecules and costimulator molecules are available from public databases. Further details of the synthesis of such a fusion molecule may be found in Mottez et al, J Exp Med. 1995 Feb. 1; 181(2):493-502, which is incorporated herein by reference. The advantage of expressing a fusion protein of the peptide and the MHC molecule is that a much higher concentration of peptides is displayed on the surface of the antigen presenting cells.

It is preferred that the antigen presenting cell is a dendritic cell because this results in a much higher level of T-cell stimulation.

As explained in the above embodiments, in the preparation of T-cells, it is preferred that there is an HLA mismatch between the antigen presenting cells and the T-cells. That is to say the antigen-presenting cells display an MHC molecule of an allele for which the donor of the T-cells is HLA negative. In some embodiments, this is achieved by obtaining the antigen presenting cells from a first individual and the T-cells from a second individual wherein the first and second individuals have an HLA mismatch.

However, in alternative embodiments, the antigen presenting cells and the T-cells are obtained from the same individual but the antigen presenting cells are transfected with polynucleotides encoding the MEC molecule of another HLA allele. In some embodiments, the polynucleotide encodes a fusion protein which encodes the MHC molecule connected to the peptide via a linker, as described above. There are numerous HLA Class I alleles in humans and the MHC molecule displayed by the antigen presenting cells, may, in principle, be of any of these alleles. However, since the HLA-A*0201 allele is particularly prevalent, it is preferred that the MHC molecule be of this allele. However, any HLA-A2 allele is usable or other alleles such as HLA-A1, HLA-A3, HLA-A 11 and HLA-A24 may be used instead.

Antigen presenting cells may be transfected as follows. A linearized DNA vector encoding the MHC molecule is transcribed in vitro to synthesise mRNA. Harvested APCs are then mixed with the mRNA and the cells are electroporated. Achievement of the appropriate level of transfection is controlled by staining with anti-MHC antibody and detection using flow cytometry.

In further embodiments of the present invention there is provided a method for preparing T-cells suitable for delivery to a patient suffering from cancer. The method comprises providing dendritic cells expressing an HLA molecule of a first HLA allele and locating a peptide in the binding groove of the HLA molecule. The peptide may or may not be a peptide of the invention. T-cells are then primed with the dendritic cells, the T-cells being obtained or obtainable from an individual who is HLA negative for a first HLA allele. As described above, the dendritic cells may either be obtained from a first donor individual and the T-cells from a second donor individual wherein the first and second donor individuals are HLA mismatched or the dendritic cells and the T-cells may be obtained from the same individual and the dendritic cells transfected with a polynucleotide encoding an MHC molecule of a different HLA allele. Indeed, such a polynucleotide may encode a fusion protein of an MHC molecule and a peptide. The advantage of using a dendritic cell, rather than a non-professional antigen presenting cell is that it results in a much higher stimulus of the T-cells

In these embodiments, it is preferred that the peptide is a cell type specific peptide, that is to say a peptide that is obtained from a protein which is only expressed, or is expressed at a much higher level (e.g. at least 10× higher concentration) in specific cells than in other cell types.

The T-cells prepared in accordance with the invention are administered to patients in order to treat cancer in the patients. In principle, the T-cells of the invention are capable of being used for the treatment of many different types of cancer including leukemia, lymphomas such as non-Hodgkin lymphoma and multiple myeloma.

Thus, in some embodiments of the present invention, pharmaceutical preparations are provided comprising a T-cell of the invention and a pharmaceutically acceptable carrier, diluent or excipient, further details of which may be found in Remmington's Pharmaceutical Sciences in US Pharmacopeia, 1984 Mack Publishing Company, Easton, Pa., USA.

As explained above, the HLA allele of the MHC molecule used to present the peptide to the T-cells is an HLA allele also expressed by the patient and therefore when the T-cells are administered to the patient, they recognize the peptide displayed on MHC molecules of that HLA allele.

In some alternative embodiments, multiple sets (e.g. 2 or 3 sets) of T-cells are provided, each T-cell being specific for a different peptide. In each case, the T-cells are allogeneic, as described above, that is to say the HLA allele of the MHC molecule on which the peptide is displayed during preparation of the T-cells is an HLA allele which is not expressed in the donor individual from whom the T-cells are obtained. The peptides may all be from the same cell specific protein or may be from different proteins but specific for the same cell type. The peptide may or may not be a peptide of the invention. In some embodiments, the multiple sets of T-cells are administered simultaneously but in other embodiments they are administered sequentially.

In the above described embodiments of the invention, reference has been made to the preparation and provision of T-cells. However, it is to be appreciated that the important feature of the T-cells is the T-cell receptor (TCR) which is displayed on the T-cells and, more specifically, the specificity of the T-cell receptor for the complex of the peptide and the MHC molecule. Therefore, in some alternative embodiments of the invention, following the preparation of T-cells as described above, the T-cell receptors of T-cells specific for a certain peptide when complexed with an MHC molecule of a particular allele are harvested and sequenced. A cDNA sequence encoding the T-cell receptor is then generated and which can be used to express the T-cell receptor recombinantly in a T-cell (e.g. the patient's own T-cells or T-cells from a donor). For example, the cDNA may be incorporated into a vector such as a viral vector (e.g. a retroviral vector), lentiviral vector, adenoviral vector or a vaccinia vector. Alternatively, a non-viral approach may be followed such as using naked DNA or lipoplexes and polyplexes or mRNA in order to transfect a T-cell.

Thus a T-cell which is “obtainable” from a donor individual includes a T-cell which is obtained recombinantly in the manner described above because the recombinantly expressed TCR is naturally produced.

Since transfected T-cells also display their endogenous TCRs, it is preferred that the T-cells are pre-selected, prior to transfection, to eliminate T-cells that would give rise to graft-versus-host disease. In some embodiments, the T-cells are pre-selected such that the specificity of their endogenous TCRs is known. For instance, T-cells are selected which are reactive with cytomegalovirus or Epstein Barr virus. In other embodiments, the T-cells are obtained from the patient and thus are naturally tolerised for the patient. This approach can only be adopted where the T-cells of the patient are healthy (e.g. for the treatment of B cell lymphomas where the T cells of the patient are normal).

In some alternative embodiments, the T-cell receptor, as a whole, is not recombinantly expressed but rather the regions of the T-cell receptor which are responsible for its binding specificity are incorporated into a structure which maintains the confirmation of these regions. More specifically, complementarity determining regions (CDRs) 1 to 3 of the T-cell receptor are sequenced and these sequences are maintained in the same conformation in the recombinant protein.

Therefore, in some embodiments of the invention, only the T-cell receptors, or a polynucleotide encoding the T-cell receptors are provided. For example, in one specific embodiment, allo-restricted T-cell lines which are reactive to a particular peptide from a protein specific for hematopoietic cells, such as CD20, when displayed by an MHC molecule of the HLA allele HLA-A*0201 are generated as described above. The T cell lines are cloned, and the T-cell receptors from one or several T-cell clones are isolated and sequenced. (In some embodiments, the alpha and beta chains of the T-cell receptor are modified so as to facilitate pairing of the introduced chains with each other over pairing with endogenous chains, as described in Kuball J, Dossett M:, Wolfl M, Ho W Y, Voss R H, Fowler C, Greenberg P D Facilitating matched pairing and expression of TCR chains introduced into human T cells. Blood. 2007 Mar. 15; 109(6):2331-8. Epub 2006 Nov. 2.) A cDNA encoding the T-cell receptor is then prepared and inserted into an expression cassette or vector. When a patient who is HLA-A*0201 positive and suffering from leukemia requires treatment, T-cells from a donor individual are transfected with the vector or expression cassette or mRNA and the T-cell receptors are expressed by and displayed on the T-cells. The T-cells are then administered to the patient in order to elicit a T-cell response to hematopoietic cells and eliminate them from the patient's body. Simultaneously, or shortly thereafter or before, the patient may be provided with an AST in order to replace the hematopoietic cells of the patient with healthy cells from a donor. If the T-cells are transfected with mRNA, the expression is transient and thus new hematopoietic cells of various lineages arise from the patient's own stem cells.

The structure of the T-cell receptor will now be described in further detail. It is to be appreciated that a naturally-occurring T-cell receptor comprises two disulfide-linked chains: an α chain and a β chain. Each of the α and β chains comprises three variable or complementarity determining regions: CDR1, CDR2 and CDR3, which make contact with the peptide-MHC complex. Of these, CDR3 is the most variable complementarity determining region (it is a hypervariable region) and is most significant in defining the binding specificity of a T-cell receptor. In order to generate a high level of variability in the sequence and binding specificity of T-cell receptors, the α and β chains are synthesised following a rearrangement of gene segments.

More specifically, the α chain is generated from the rearrangement of over 70 V gene segments and 61 J gene segments such that one V gene segment is joined to one J gene segment. The J gene segment is also joined to a C gene segment.

The β chain is generated from the rearrangement of 52 V gene segments, 2 D segments and 13 J gene segments such that one V gene segment is joined to one D gene segment which is, in turn, joined to one J gene segment. The J gene segment is also joined to one of two C gene segments: Cb1 or Cb2 (the alignment between the peptide sequences encoded by these genes is shown in FIG. 19). In the case of the β chain, the D, J and C genes segments exist as two clusters such that the D gene segment in the second cluster (i.e. the D2 gene) may only be joined to one of the 7 J gene segments in the second cluster (i.e. one of the J2 genes).

Further variability of α and β chains arises due to variable boundary recombinations at the junctions of the V, D and J genes. It is also to be noted that the sequence of CDRs 1 and 2 is encoded by the V gene segment while the sequence of CDR3 is encoded by the junction formed between the V and J gene segments (in the case of the α chain) and the junction formed between the V, D and J gene segments (in the case of the β chain). Accordingly, it will be observed that once the V gene segment (which encodes CDRs 1 and 2) and the sequence of CDR3 of an α or β chain are defined, the the binding specificity of the T-cell receptor is also defined. Furthermore, the V and J domains outside the CDR3 are not modified and are defined according to the corresponding selected germline encoded regions. The C domain is joined to the J domain.

For example, FIG. 17 shows the sequence of a T-cell receptor α chain encoded by the V gene V12-2, the J gene J43 and the Ca gene. The peptide sequences encoded by each gene segment are shown. As can be seen, CDRs 1 and 2 are encoded by the V gene and CDR3 is encoded by the junction between the V and J gene segments.

FIG. 18 shows the sequence of a T-cell receptor β chain encoded by the V gene V6-5, the D1 gene, the J gene J2-7 and the Cb1 gene. As can be seen CDRs 1 and 2 are encoded by the V gene and CDR3 is encoded by the junction between the V and J gene segments (with the sequence encoded by the D gene being substituted).

In this specification, the CDR3 region as well as the numbering of the different domains is done according to the IMGT database whereby the first C residue belongs to the Vbeta domain and the last F residue belonging to the J-domain (i.e. they are germline-encoded residues). The amino acids in between are part of the CDR3 region. Since the length of the region can vary from one clone to the other, the numbering of the amino acids is done as follows: +aa1=first amino acid after the cysteine residue (C) (from the Vbeta), +aa2=second amino acid after C, etc. These amino acids can also be designated according to their location from the phenylalanine residue (F) (of the J-domain): −aa-1=first amino acid before F, etc.

In some embodiments the target antigen is SEQ. ID NO. 18 which is a peptide of CD20. That is to say the T-cell receptor is capable of binding SEQ. ID NO. 18 when it is in complex with an HLA-A molecule, preferably HLA-A*0201. In some of these embodiments, the CDR3 region of the β chain of the T-cell receptor has a glycine residue at position +5. In some embodiments, the CDR3 regions of these T-cell receptors are encoded by J-2 gene segments, such as segments J2-7, J2-5, J2-1, J2-2 (i.e. J2-2*01 but not J2-2P*01), J2-3 or J2-4, the peptide sequences of which are shown in Table 6. In some embodiments, the amino acid at position −1 of the CDR3 region is an aromatic amino acid, e.g. tyrosine or phenylalanine. In preferred embodiments, positions −2 and −1 of the CDR3 regions of these T-cell receptors are glutamine and tyrosine, respectively. In particularly preferred embodiments position −3 of the CDR3 region is glutamic acid. The CDR3 regions of the β chains of the T-cell receptors of some embodiments are between 9 and 12 amino acid residues, with 11 amino acid residues being most preferred. It is particularly preferred that the CDR3 region of the β chain of the T-cell receptors conforms to a consensus sequence selected from SEQ ID NO. 167, 168, 169 and 189. It is preferred in these embodiments that the sequence of the CDR3 region is selected from one of SEQ ID NOS. 148 to 155 or 170 to 174. While it is not essential to the invention, in many embodiments the T-cell receptor or receptor fragment comprises an α chain and it is preferred that this α chain binds a peptide of the CD20 protein such as SEQ. ID NO. 18. For example, the CDR3 regions of the α chains may be selected from SEQ ID NOS. 175 to 188 or 190.

As explained above, it is the CDR3 of a T-cell receptor that primarily defines its binding specificity. Once the CDR3 sequence is selected the sequence of the remainder of the T-cell receptor may be constructed from the germline-encoded sequences of known T-cell receptors (e.g. as shown in FIGS. 17 and 18) or by incorporating sequences encoded by the V, D, J and C gene sequences which are known in the art (see, for example the IMGT reference directory and Folch, G. and Lefranc, M.-P., Exp. Clin. Immunogenet., 17, 107-114 (2000)). Thus, the remainder of the T-cell receptor may be constructed in the same way as other T-cell receptors, as described above, in order to provide the specificity for SEQ. ID NO. 18.

It is also to be understood that in further embodiments of the present invention, the sequences of the components of the T-cell receptor (e.g. CDR3 sequences) described herein are further adapted, for example to increase binding affinity. Thus in some embodiments, one or more amino acids of the T-cell receptor sequences are omitted or are substituted for a similar amino acid. A similar amino acid is one which has a side chain moiety with related properties and the naturally occurring amino acids may be categorized into the following groups. The group having basic side chains: lysine, arginine, histidine. The group having acidic side chains: aspartic acid and glutamic acid. The group having uncharged polar side chains: asparagine, glutamine, serine, threonine and tyrosine. The group having non-polar side chains: glycine, alanine, valine, leucine, isoleucine, proline, phenylalanine, methionine, tryptophan and cysteine. Therefore it is preferred to substitute amino acids within these groups.

Kits comprising suitable T-cell receptors (or the components thereof having binding specificity) or nucleic acid molecules encoding such receptors for use in the above-described methods are also provided in some embodiments

In this invention the T-cells are generally CD8⁺ T lymphocytes. However, in some embodiments, both CD8⁺ and CD4⁺ T cells are transfected with polynucleotides encoding a TCR as this results in a more powerful immune response.

It is preferred that the T-cells are transiently transfected with mRNA encoding the TCR as this avoids the risks associated with an irreversible genetic manipulation. An example of transfection of mRNA of T-cell receptors is reported in Schaft N, Dorrie J, Muller I, et al. A new way to generate cytolytic tumor-specific T cells: electroporation of RNA coding for a T cell receptor into T lymphocytes. Cancer Immunol Immunother. 2006; 55:1132-1141. However, T-cells can also be transfected permanently for example by using a retrovirus as is reported in Morgan et al Science. 2006 Oct. 6; 314(5796):126-9. Procedures for isolating and cloning T-cell receptors (TCRs) are also reported in WO02/44207 and will be summarized as follows. The TCR usage in the CTL clones is determined using (i) TCR variable region-specific monoclonal antibodies and (ii) RT-PCR with primers specific for Vα and Vβ gene families. A cDNA library is prepared from poly-A mRNA extracted from the CTL clones. Primers specific for the C-terminal portion of the TCR α and β chains and for the N-terminal portion of the identified Vα and β segments are used. The complete cDNA for the TCR α and β chain is amplified with a high fidelity DNA polymerase and the amplified products cloned into a suitable cloning vector. The cloned α and β chain genes may be assembled into a single chain TCR by the method as described by Chung et al (1994) Proc. Natl. Acad. Sci. USA 91, 12654-12658. In this single chain construct the VαJ segment is followed by the VβJ segment, followed by the Cβ segment followed by the transmembrane and cytoplasmic segment of the CD3ζ chain. This single chain TCR is then inserted into a retroviral expression vector (a panel of vectors may be used based on their ability to infect mature human CD8⁺ T lymphocytes and to mediate gene expression: the retroviral vector system Kat is one preferred possibility (see Finer et al (1994) Blood 83,43)). High titre amphotrophic retrovirus are used to infect purified CD8⁺ T lymphocytes isolated from the peripheral blood of tumor patients following a protocol published by Roberts et al (1994) Blood 84, 2878-2889, incorporated herein by reference. Anti-CD3 antibodies are used to trigger proliferation of purified CD8⁺ T-cells, which facilitates retroviral integration and stable expression of single chain TCRs. The efficiency of retroviral transduction is determined by staining of infected CD8+ T-cells with antibodies specific for the single chain TCR. In vitro analysis of transduced CD8⁺ T cells establishes that they display the same tumor specific killing as seen with the allo-restricted CTL clone from which the TCR chains were originally cloned. Populations of transduced CD8⁺ T cells with the expected specificity may be used for adoptive immunotherapy of the tumor patients. Patients may be treated with in between 10⁸ and 10¹¹ (preferably between 10⁹ and 10¹⁰) autologous, transduced CTL.

Other suitable systems for introducing genes into CTL are described in Moritz et al (1994) Proc. Natl. Acad. Sci. USA 91,4318-4322; Schaft et al Cancer Immunol Immunother. 2006 September; 55(9):1132-41; and Clay et al J. Immunol. 1999, 163:507, each of which is incorporated herein by reference. Eshhar et al (1993) Proc. Natl. Acad. Sci. USA 90, 720-724 and Hwu et al (1993) J. Exp. Med. 178,361-366 also describe the transfection of CTL.

It is particularly preferred that the donor of any stem cell transplant which is implanted as part of the therapeutic intervention is HLA negative for the HLA allele for which the T-cells are responsive when displayed by the peptide complexed with MHC. For example, if the T-cells are specific for a peptide when displayed by MHC molecules as HLA allele HLA-A*0201 then it is preferred that the donor of the tissue is HLA-A*0201 negative so that the donor tissue cells repopulate the patient. Thus the T-cells selectively destroy T-cells in the patient of that tissue, including the malignant cells but the cells of the donor tissue remain unharmed. However, this is not always necessary in embodiments where the genetically engineered T cells only transiently express the T cell receptor that confers target cell killing. Transfection of T cell receptors by use of mRNA encoding the receptor is an example of such transient expression.

While the above described embodiments of the invention have referred to T-cell receptors, it is to be appreciated that also included in the invention are molecules and complexes of molecules which function in the same way as T-cell receptors such as genetically engineered 3-domain single chain T-cell receptors as described in Chung et al Proc Natl Acad Sci USA, 1994 Dec. 20; 91(26):12654-8. It is to be appreciated, however, that such equivalents to T-cell receptors operate in the same fashion, namely by being responsive to a peptide when displayed by an MHC molecule of a particular HLA allele.

In further embodiments, the T-cell receptor is a chimeric T-cell receptor. The synthesis of chimeric T-cell receptors is disclosed in U.S. Pat. No. 6,410,319 which is incorporated herein by reference.

Among the most successful immunotherapy of cancer is antibodies directed against wild-type proteins with cell type-restricted expression. Anti-CD20 (RituxiMab) is today standard therapy in non-Hodgkins B cell lymphoma. In addition, a number of other antibodies are clinically tested in hematological malignancies, such as anti-CD2, anti-CD52 and anti-CD22 (Fanale M A, Younes A: Monoclonal antibodies in the treatment of non-Hodgkin's lymphoma., Drugs. 2007; 67(3):333-50. Review.) All of these agents target proteins that are expressed in healthy tissue as well as on cancer cells. Since the molecule is not unique to a given tumor, a single specificity can be used in all patients with the same cancer type, whereas toxicity is limited by the tissue-restricted expression of the target. Due to the capacity of anti-CD20 antibodies to induce apoptosis in normal as well as cancerous B cells, anti-CD20 therapy has been successfully introduced in therapy of non-cancerous diseases wherein dysfunctional B cells are targeted. Thus, the efficacy of anti-CD20 treatment has been demonstrated in a number of autoimmune diseases, including multiple sclerosis, rheumatoid arthritis, systemic lupus erythematosus, immune thrombocytopenic purpura, chronic cold agglutinin disease, IgM-mediated neuropathies and mixed cryoglobulinemia. Such treatments are also effective in relation to inherited immunodeficiencies, or bone marrow failure, in particular aplastic anemia or paroxysmal nocturnal hemoglobinuria.

In analogy with this, the present invention's use of T cells or T cell receptors targeted at cell type specific peptides can be useful for therapy of non-cancerous diseases caused by dysfunctional hematopoietic cells. In particular, a patient's diseased immune system is replaced with a new, healthy immune system from a donor. Due to the reduced incidence of GVHD in the therapies of the present invention, such treatments would be better tolerated and would more efficiently eradicate the diseased immune system compared with AST as currently used. Moreover, the cell type specific proteins can be targets for T cell therapy of the present invention at an earlier point than for prior art antibody-therapy during cellular differentiation. This is because peptides from proteins being synthesized intracellularly but not yet expressed on the cell surface—or expressed at low levels—can be presented on MHC molecules and therefore evoke T cell responses.

These embodiments of the present invention can be applied in a clinical setting. T cells reactive with CD20/HLA-A2, are primarily useful in a setting where an HLA-A2+ patient has been transplanted with stem cells from an HLA-A2 negative donor. The CTL response is then host-specific, allowing the severe side effects of B cell depletion to be overcome. Whereas a complete HLA-match provides the optimal outcome in AHSCT, mismatch on a single MHC antigen between donor and recipient is permitted. T cell depletion reduces the risk of GVHD, but also increases the risk of graft failure and relapse. Infusion of targeted CTLs might provide sufficient GVL- and engraftment support to allow a T cell-depleted stem cell graft. Another option is to clone the A2/CD20-reactive TCRs for genetic transfer into T cells from the patient or a fully matched donor. T cell receptors that recognize peptide/HLA-A2 complexes expressed only on hematopoietic cells are potentially useful in 50% of Caucasians. Accordingly, allo-restricted T cells may induce GVL without GVHD. Since CD20 is found on the majority of B-cell malignancies, immunotherapy with T cells expressing allo-restricted, CD20-specific TcRs have a wide applicability.

While the above described embodiments relate to the preparation of compositions for the treatment of cancer, in alternative embodiments the products and kits of the invention are used for the treatment of diseases related to a (non-cancer related) dysfunction in hematopoietic cells.

### EXAMPLES

Materials and Methods

pCIpA₁₀₂ HLA-A*0201 Construct and In Vitro Transcription of mRNA

Full length HLA-A*0201 cDNA inserted between EcoRI and XhoI sites of a pcDNA3.1 vector (Invitrogen, Groningen, Netherlands) was provided as a gift of Dr. C Britten (Johannes Gutenberg-University of Mainz, Mainz, Germany). The pCIpA₁₀₂ expression vector was obtained from Prof. G. Gaudernack (Saeboe-Larssen, S., E. Fossberg, and G. Gaudernack, mRNA-based electrotransfection of human dendritic cells and induction of cytotoxic T lymphocyte responses against the telomerase catalytic subunit (hTERT). J Immunol Methods, 2002. 259(1-2): p. 191-203). A BglII/NotI fragment containing the HLA-A*0201 cDNA together with the CMV and T7 promoters was isolated and inserted between the respective cut sites in pCIpA₁₀₂ to generate pCIpA₁₀₂ HLA-A*0201.

For in vitro transcription of mRNA, the construct was linearized with MfeI and purified by using Wizard SV Gel and PCR Clean-Up System (Promega, Madison, Wis., USA) as described in the kit manual. They were then transcribed in vitro using the RiboMAX Large Scale T7 RNA production system (Promega) using a modified reaction mix containing increased concentrations of rGTP and cap analogs, as described before (Saeboe-Larssen, S., E. Fossberg, and G. Gaudernack, mRNA-based electrotransfection of human dendritic cells and induction of cytotoxic T lymphocyte responses against the telomerase catalytic subunit (hTERT). J Immunol Methods, 2002. 259(1-2): p. 191-203). The mRNA was extracted by phenol:chloroform:isoamyl alcohol (25:24:1) and chloroform:isoamyl alcohol (24:1) before precipitation with ethanol. The mRNA was dissolved in nuclease free water and stored in aliquots at −70° C. until further use. Spectrophotometry was used to quantitate the amount of mRNA produced, and generally 1-2.6 mg mRNA was produced per reaction.

### Generation of Monocyte-Derived Dendritic Cells (moDC) for Transfection Experiments and Induction of Pentamer Positive Cells (for Use in Examples 1 to 3)

Peripheral blood mononuclear cells (PBMC) were isolated from full blood from HLA-A*0201-negative healthy donors. The PBMC were resuspended in serum free CellGro DC medium (CellGenix, Germany) at 20×10⁶ cells per 3 ml and seeded in 6-well plates, 3 ml cell suspension per well. The cells were left to adhere for 2 hours at 37° C., 5% CO₂. After two hours the wells were washed twice with serum-free RPMI 1640. The cells removed by washing (the non-adherent cells) were frozen and were later thawed for use as responder cells as explained below. To the cells remaining in the wells (the adherent cells), 2.5 ml CellGro DC-medium with 500 U/ml IL-4 (Peprotech EC Ltd., London, United Kingdom) and 800 U/ml GM-CSF (Berlex Laboratories, Inc., Richmond, Calif., USA) were added.

On day 2, 2.5 ml medium containing 1000 U/ml IL-4 and 1600 U/ml GM-CSF, was added. On day 4, 2.5 ml medium was removed from each well and replaced with 2.5 ml fresh medium containing 1000 U/ml IL-4 and 1600 U/ml GM-CSF. On day 6, 1 ml medium was removed from each well, and replaced with 1 ml fresh medium containing 10 ng/ml TNF-alpha (Peprotech EC, Ltd.) and 50 ng/ml lipopolysaccharide for E. coli (LPS, Sigma-Aldrich) to achieve final concentrations of 2 ng/ml and 10 ng/ml, respectively. At day 7, after maturation for 24 hours, the cells were harvested and transfected.

### mRNA-Transfection with HLA-A*0201 (for Use in Examples 1 and 2)

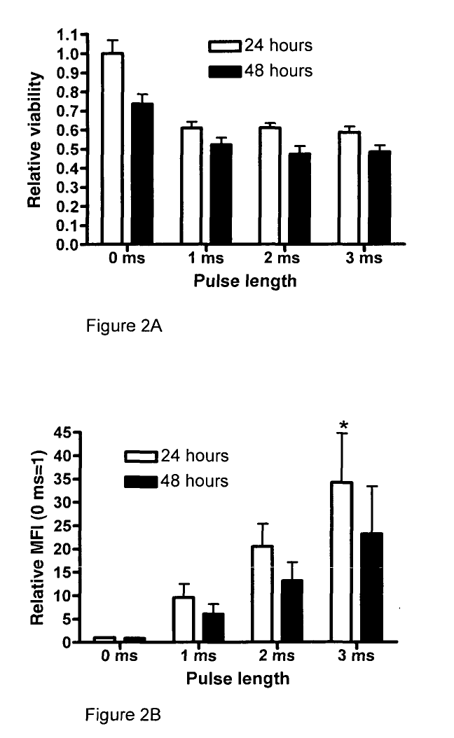

For transfection of moDC, the cells were harvested, washed once and resuspended at concentrations of 10-20×10⁶ cells/ml in RPMI 1640 without serum and antibiotics. The growth medium containing maturation factors was saved and used for growing the cells after transfection. The washed cells were aliquoted in 400 μl, and 20-40 μl mRNA or water (for the controls) was added to the cells. The cells were transferred to a 4 mm electroporator cuvette and electroporated at 1250 V/cm for 1, 2 or 3 ms (as indicated in FIGS. 2A and 2B), with a BTX ECM 830 square wave electroporator.

For transfection before maturation, the cells were harvested on day 6 after isolation of monocytes. The MoDC were then washed and resuspended in serum-free RPMI and transfected as described above. Fresh DC-medium containing 2 ng/ml LPS and 10 ng/ml TNF-alpha was prepared and the cells were seeded in this following transfection, and grown for 24 hours before analysis.

Transfection of EBV-transformed lymphoblastoid cells (EBV-LCL) was performed as described for moDC, with some exceptions: 200 μl cell suspension was distributed to each tube, and 10-20 μl mRNA added to each sample. The EBV-LCL were electroporated in a 2 mm cuvette at 1250 V/cm for 1 ms. Following electroporation and resting on ice, the cells were grown in RPMI containing Penicillin-Streptomycin Solution (referred to as RPMI P/S; Sigma-Aldrich, St. Louis, Md., USA) yielding final concentrations of 50 U/ml Penicillin G and 50 μg/ml streptomycin, supplemented with 10% fetal calf serum (FCS).

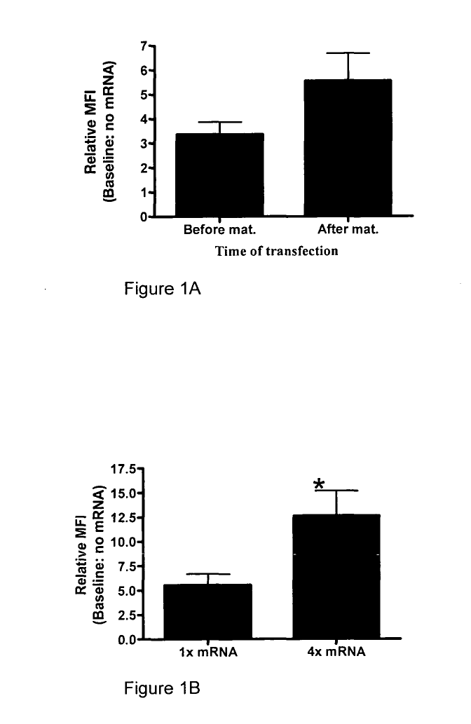

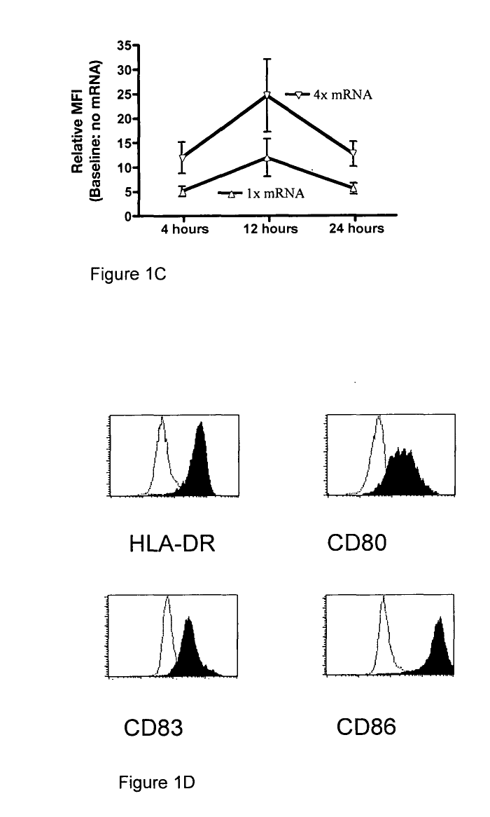

At 4, 12, 24 or 48 hours after transfection (see FIG. 1C and FIG. 2) the MoDC were harvested and stained with antibody against HLA-A*0201. MoDC were also stained with antibodies against CD80, CD83, CD86, and HLA-DR and analyzed by flow cytometry to verify that the cells had a fully mature phenotype (FIG. 1D).

### Peptides and Pentamers (for Use in Examples 3 to 5)

The HLA-A*0201 binding peptide ELAGIGILTV (referred to as MART-1 peptide, ProImmune Ltd., Oxford, United Kingdom) is a modification of the native MART-1 (26-35) peptide EAAGIGILTV containing an A27L substitution, with enhanced immunogenicity compared to the native peptide, as shown in Valmori D et al, Generation of Specific Tumor-Reactive CTL In Vitro by Selected Melan-A/MART-1 Immunodominant Peptide Analogues. J Immunol 1998; 160(4):1750-1758. NLVPMVATV (referred to as CMV peptide, ProImmune Ltd.) derived from human cytomegalovirus pp65 (495-503) was used as a HLA-A*0201 binding control peptide.

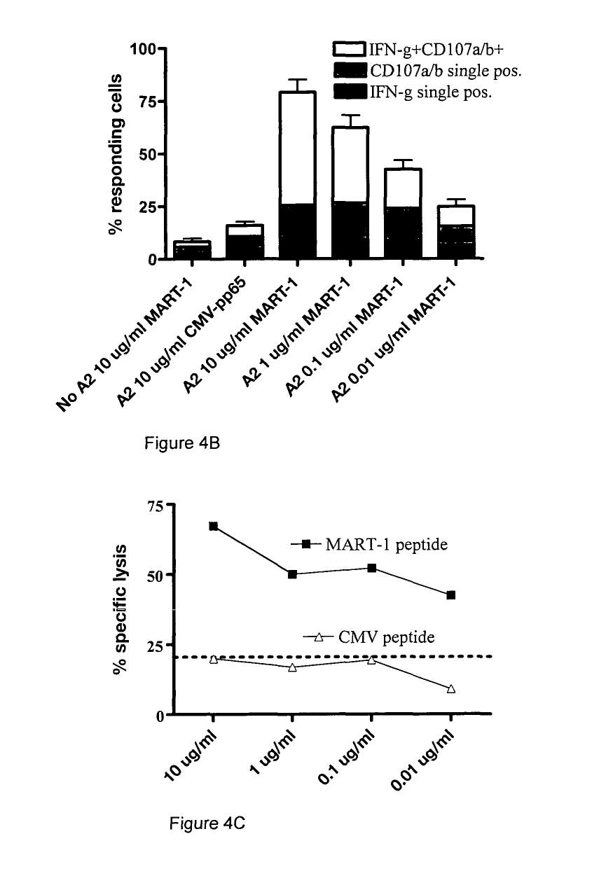

Peptide-MHC pentamers corresponding to the peptides were used for detection and sorting of antigen-specific T cells, and HLA-A*0201-ELAGGILTV (referred to as MART-1 pentamer, ProImmune Ltd.) and HLA-A*0201-NLVPMVATV pentamers (referred to as CMV-pentamer, ProImmune) were purchased unlabeled, and labeled with fluorescent APC and PE fluorotags (ProImmune Ltd.) as indicated in FIGS. 3 and 4.

Induction of Alloreactive Antigen-Specific T Cell Lines

MoDC transfected with HLA-A*0201 mRNA were harvested 18 hours after transfection and resuspended in 1-2 ml of serum-free DC-medium containing 50 μg/ml peptide, such as MART-1 or peptide #19. The cells were incubated at 37° C., 5% CO₂ for 4 hours, then washed once in serum-free RPMI.

The frozen, non-adherent cells were thawed and washed for use as responder cells.

Stimulator and responder cells were seeded at a stimulator to responder (S:R) ratio of 1:10, with 3.0×10⁶ responders per well in serum-free DC medium containing 10 ng/ml IL-7 (PeproTech EC Ltd.), 50 pg/ml IL-12 (PeproTech EC Ltd.), and PPD at 1 μg/ml.

On day 7 half of the medium was replaced with fresh serum-free DC medium containing 20 ng/ml IL-7.

On day 12 the responder cells were harvested and analyzed for the presence of pentamer positive cells. The cells were either sorted or restimulated.

Restimulation was done with autologous EBV-LCL transfected with HLA-A*0201 mRNA 12-24 hours prior to stimulation, harvested and resuspended in 1-2 ml of serum-free X-Vivo 20 (Cambrex) containing the same peptide as added on day 0 at 20 μg/ml, and incubated for 4 hours at 37° C., 5% CO₂. Following incubation, the EBV-LCL were washed once in serum-free RPMI.

For restimulation, the responder cells were resuspended in X-Vivo 5% HPS (human pooled serum) at 6×10⁶ cells/ml.

0.25 ml per well of each of the stimulator and responder cell suspensions were seeded in a 48-well plate, to achieve a stimulator:responder ratio of 1:5. Two days after restimulation (day 14), 0.5 ml X-Vivo 5% HPS containing 20 U/ml IL-2 (R&D Systems, Minneapolis, Minn., USA) was added to each well.

From day 12 of co-culture with stimulator cells, the responders were harvested, counted and analyzed for the presence of pentamer positive cells weekly. The responder cells were either restimulated, as described above, or the pentamer positive cells were sorted and expanded as described below.

### Sorting Pentamer Positive Cells (for Example 3)

For sorting, the cells were harvested and spun down, and resuspended in a small volume containing the unlabeled MART-1 pentamer. For staining before sorting, 1/16 of the amount recommended by the manufacturer was used. Briefly, the cells were incubated 15 minutes on room temperature, washed once with PBS with 2% FCS, resuspended in a volume containing the fluorotag and anti-CD8 antibody. Cells were either sorted by magnetic beads or by flow cytometry. For magnetic bead sorting, a fluorotag conjugated to PE was used for labeling pentamer positive cells, and the pentamer positive cells were isolated using MACS Anti-PE MicroBeads (Miltenyi Biotec, Bergisch Gladbach, Germany) and run over a MACS MS Column (Miltenyi Biotec). Labeling with Anti-PE MicroBeads and cells separation was performed according to the manufacturers instructions. As indicated in FIG. 3B, the cells were run over two subsequent columns to achieve the highest purity.

A FACS Aria Cell sorter (BD Biosciences) was used to directly sort the cells following labeling with pentamer, fluorotag and anti-CD8 antibody. The cells were first enriched for pentamer positive cells using the Yield mode, and subsequently resorted to high purity using a more stringent Purity mode.

### Antigen-Independent Expansion of Purified Pentamer Positive Cells (for Example 3)

FIG. 3C demonstrates the expansion of a cell line after sorting to purity higher than 98%. For antigen-independent expansion of sorted pentamer positive cells, the cells were seeded with allogeneic PBMC from two or three donors as feeder layer (allo-FL), in X-Vivo 20 5% HPS with 50 U/ml IL-2, 2 ng/ml IL-15 and 1 μg/ml Phytohaemagglutinin (PHA; Remel, Lenexa, Kans., USA). The cells were seeded in 96-, 24- and 6-well plates, depending on the number of cells and at which stage of expansion they were. In 96-well plates, 1000, 5000, 10000 or 50000 pentamer positive cells were seeded with 0.5-1.0×10⁶ allo-FL per well. In 24-well plates, 0.25×10⁶ pentamer positive cells were seeded with 2.0-3.0×10⁶ allo-FL per well. In 6 well plates, 1.0×10⁶ pentamer positive cells were seeded with 7.5×10⁶ allo-FL per well. The cells were split and/or supplemented with fresh medium containing IL-2, IL-15 and PHA, when needed. No PHA was added the last day before restimulation.

For quantification of cell expansion, the cells were harvested, stained with MART-1 pentamer or CMV pentamer, and antibodies against CD3 and CD8. The exact number of pentamer positive cells was determined by adding a fixed number of beads when analyzing the cells by flow cytometry.

### Functional Assays (for Examples 4 to 6)

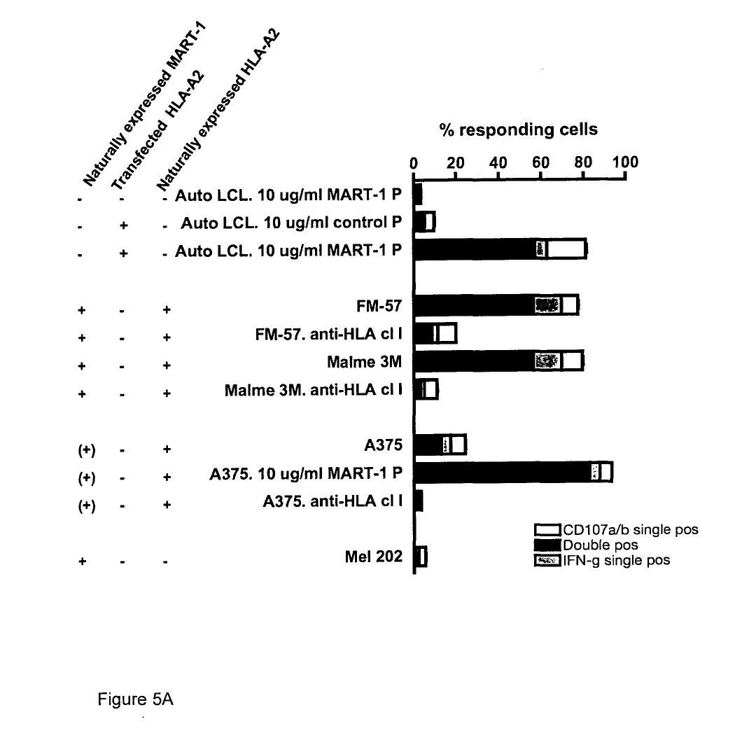

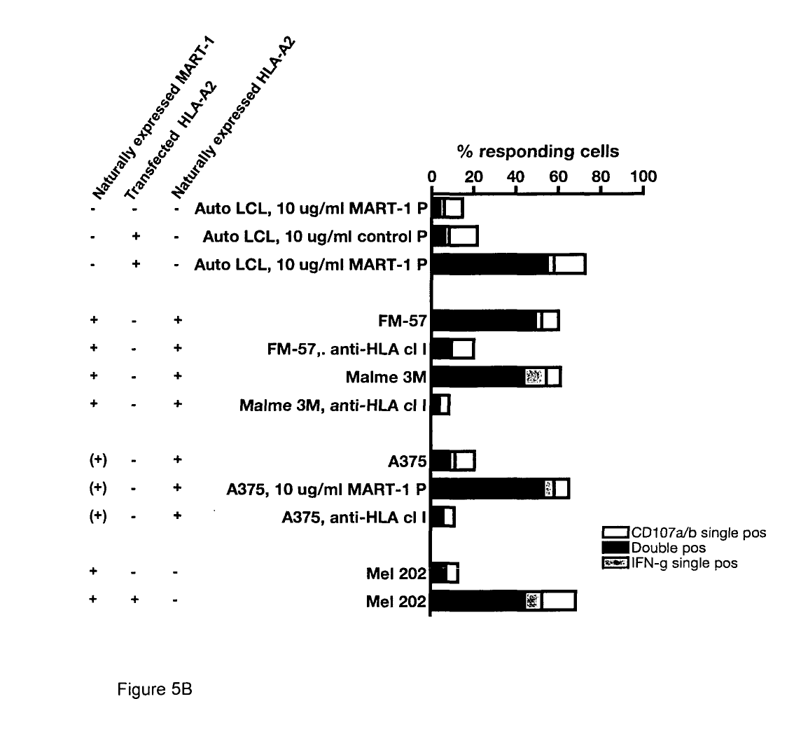

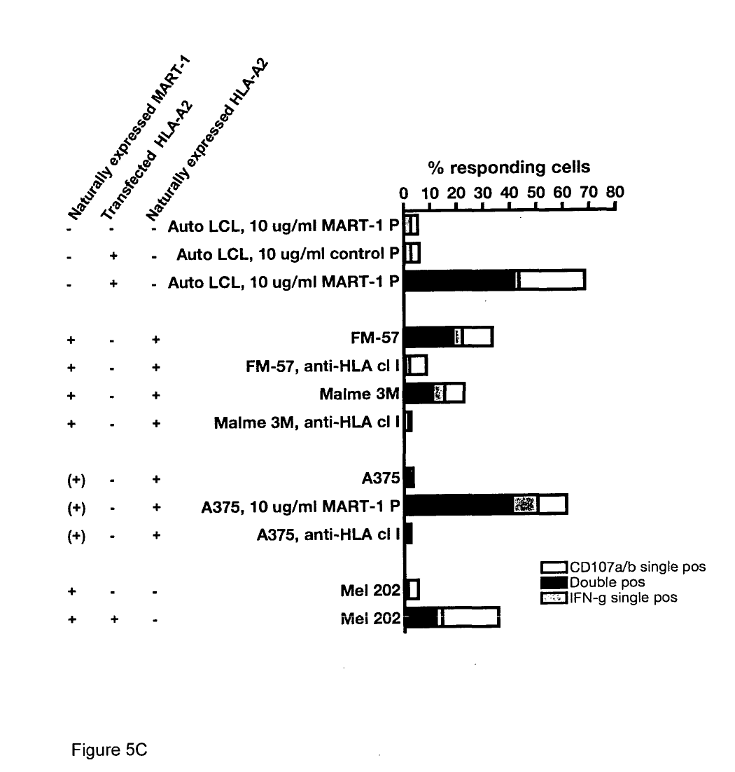

The pentamer positive T cells were tested for the ability to produce interferon-gamma and lyse target cells using a modification of assays previously described in Betts, M. R., et al., Sensitive and viable identification of antigen-specific CD8+ T cells by a flow cytometric assay for degranulation. J Immunol Methods, 2003. 281(1-2): p. 65-78 and Openshaw, P., et al., Heterogeneity of intracellular cytokine synthesis at the single-cell level in polarized, T helper 1 and T helper 2 populations. Journal of Experimental Medicine, 1995. 182(5): p. 1357-67. Prior to the stimulation, the cells were harvested, counted and resuspended in X-Vivo 20 5% HPS with 2 ng/ml IL-15 and rested for 2-3 days at 1.75×10⁶ cells/well in 24-well plates. The stimulation was carried out, with minor modifications, as previously described in Betts, M. R., et al., HIV nonprogressors preferentially maintain highly functional HIV-specific CD8+ T cells. Blood, 2006. 107(12): p. 4781-9. On the day of stimulation, the responder cells were harvested, counted and resuspended at 1.0×10⁶ cells/ml in X-Vivo 20. The target cells were harvested and prepared with and without HLA transfection and with and without peptide as indicated in FIGS. 4 and 5. For transfection, cells were electroporated the day before stimulation, as described above. On the day of the assay, cells were harvested and pulsed for 2 hours with MART-1 or CMV peptide, as indicated in FIGS. 4 and 5. All target cell lines were washed, counted and resuspended at 0.5×10⁶ cells/ml in serum-free X-Vivo 20. 96-well round-bottom polypropylene plates were used for the stimulations, and 100 μl target cell suspension was distributed per well, and blocking anti-HLA-ABC antibody W6/32 was added at 100 μg/ml in some cases as indicated. The cells were incubated for 10 minutes at 37° C. Following the brief incubation, 5 μl of a 10 μg/ml solution of each of the anti-CD 107a and anti-CD 107b antibodies was added together with 1 μl of a 2 μg/ml Brefeldin A (Sigma-Aldrich) solution and 1 μl of a 2 mM Monensin A (Sigma-Aldrich) solution. Finally, 100 μl responder cell suspension was added to each well. The final responder:stimulator ratio was 2:1 for these assays. The cells were incubated for 5 hours at 37° C., 5% CO₂. Following incubation, the cells were harvested, fixed with paraformaldehyde (PFA), permeabilized with a solution containing saponin and stained for the presence of intracellular IFN-γ. Briefly, the cells were transferred to 96-well V-bottom polystyrene plates, stained for surface markers, washed twice and resuspended in a 2% PFA solution. The cells were then left over night in the fridge, before further permeabilization and intracellular staining. The next day, the cells were washed once with PBS containing 2% FCS and 0.5% saponin (referred to as PBS/FCS/sap) and resuspended in PBS/FCS/sap permeabilizing reagent and incubated 10 minutes on room temperature. Following permeabilization, the cells were spun down and the supernatant removed. Anti-IFN-γ antibody was added, and the cells were incubated for 20 minutes on room temperature. The cells were then washed twice in PBS/FCS/sap and once in PBS with 2% FCS and 0.5 mM EDTA. For the analysis, the cells were resuspended in

PBS 2% FCS 0.5 mM EDTA and analyzed on a FACS Calibur flow cytometer (BD Biosciences).

Antibodies and Flow Cytometry

CD3 FITC, CD3 APC, CD3 PerCP-Cy5.5, CD8 FITC, CD8 APC, CD8 PerCP-Cy5.5, CD80 PE, CD86 FITC, HLA-DR PerCP, IFN-γ PE, CD107a FITC, CD107b FITC (BD Biosciences), HLA-A*0201 PE (ProImmune Ltd.), CD80 Alexa 647, CD83 Alexa 488 and IFN-γ FITC (Serotec Ltd, Oxford, UK) were all purchased pre-conjugated. Purified antibodies against HLA-A*0201 (Serotec Ltd), CD 107a (BD Biosciences) and CD107b (BD Biosciences) were conjugated in the inventors' laboratory to Alexa 647 (Molecular Probes Inc., Eugene, Oreg., USA). The cells were stained with an optimally titrated amount of antibody, washed twice in PBS with 2% FCS and 0.5 mM EDTA, and resuspended in PBS 2% FCS 0.5 mM EDTA before analysis. The cells were analyzed on a FACS Calibur Flow cytometer (BD Biosciences), and data was acquired and analyzed using CellQuest Pro Software (BD Biosciences).

### Melanoma Cell Lines (for Example 5)

The melanoma cell lines A375 and Malme 3M were obtained from American Type Culture Collection (ATCC, http://www.attc.org). A375 is HLA-A*0201 positive, and shows weak expression of MART-1 (shown in de Vries T J, Fourkour A, Wobbes T, Verkroost G, Ruiter D J, van Muijen GNP. Heterogeneous Expression of Immunotherapy Candidate Proteins gp100, MART-1, and Tyrosinase in Human Melanoma Cell Lines and in Human Melanocytic Lesions. Cancer Res 1997;57(15):3223-3229). Malme 3M is reported to be HLA-A*0201 positive and expresses MART-1. FM-57 and Mel 202 was obtained from The European Searchable Tumour Line Database (ESTDAB, http://www.ebi.ac.uk/ipd/estdab/). FM-57 is HLA-A*0201 positive and expresses MART-1, whereas Mel 202 expresses MART-1, but is HLA-A*0201 negative.

### Leukemia Cell Lines and Protein Expression (for Example 6)

The leukemia cell lines THP-1, JVM-2, Nalm-6, REH and ML-2 were all obtained from Deutsche Sammlung von Mikroorganismen and Zellkulturen GmbH (DSMz, http://www.dsmz.de). THP-1 is an HLA-A*0201 positive acute monocytic leukemia. JVM-2 is an HLA-A*0201 B-prolymphocytic leukemia. Nalm-6 is an HLA-A*0201 positive B cell acute lymphoblastic leukemia. REH is an HLA-A*0201 negative B cell acute lymphoblastic leukemia. ML-2 is an HLA-A*0201 positive acute myleomonocytic leukemia. Expression of HLA-A*0201 and CD20 as referred to in Example 6 was confirmed in our laboratory by flow cytometric analysis. The cells were stained with CD19 FITC (BD), CD20 PE (BD) and HLA-A*0201 Alexa 647 (conjugated in the inventor's laboratory, as mentioned above) and analyzed on a FACS Calibur flow cytometer (BD) using Cell Quest Pro (BD) software for data analysis.

### Example 1

### Optimizing mRNA-Transfection of MoDC: Effects of Transfecting Cells Before or After Maturation, and of Increasing Concentrations of mRNA

Monocyte-derived dendritic cells (moDC) were transfected before or after maturation, and were analyzed 24 hours after transfection. Maturing the moDC before transfection led to an increased level of expression of HLA-A*0201 at 24 hours following the transfection. Relative mean fluorescence intensity (MFI) was calculated by dividing the MFI of the cells electroporated with mRNA by the value of cells electroporated without mRNA. (n=4). The results are shown in FIG. 1A.

MoDC were transfected after maturation with increasing concentrations of mRNA. Increasing the concentration of mRNA 4 times led to significantly higher expression of HLA-A*0201 (*=p<0.05). (n=4). The results are shown in FIG. 1B.

Following maturation, moDC were transfected with HLA-A*0201 mRNA at two different concentrations, and analyzed at t=4, 12 and 24 hours post transfection. The results are shown in FIG. 1C. Expression of HLA-A*0201 is displayed as MFI relative to the MFI of cells transfected with no mRNA. Expression of HLA-A*0201 could be detected already 4 hours post-transfection, was increased at 12 hours, and present at 24 hours at levels similar to 4 hours. (n=4).

MoDC transfected with HLA-A*0201 mRNA after maturation, were assessed for levels of the maturation markers HLA-DR, CD80, CD83 and CD86. Representative data from 1 of 4 experiments are shown in FIG. 1D. The histograms display the MFI of the respective maturation markers. The filled line (black) represents the marker, whereas the open line represents the relevant antibody isotype controls. High levels of the maturation markers were expressed in the moDC.

### Example 2

### Optimizing mRNA-Transfection of MoDC: Effect of Increased Pulse Length

MoDC were transfected with HLA-A*0201 mRNA after maturation for 24 hours, and the expression of HLA-A*0201 and cell viability was assessed at 24 and 48 hours post transfection.

Viability was measured by labeling the cells with propidium iodide prior to flow cytometric analysis. A fixed number of beads were added to each sample, allowing accurate calculation of the number of cells in the sample. Percentages of viable cells were calculated by dividing the absolute number of viable cells in the samples transfected for 1, 2 or 3 ms, respectively, by the absolute number of viable cells in the samples that had not received an electric impulse. Increasing the pulse length did not significantly affect the viability of the moDC. (n=4). The results are shown in FIG. 2A.

The expression of HLA-A*0201 was analyzed by flow cytometry and the results are shown in FIG. 2B. Relative MFI was calculated by comparing the MFI of the cells electroporated at 1, 2 and 3 ms to the MFI of cells not electroporated (0 ms) at 24 and 48 hours after transfection. Increasing the pulse length from 1 to 3 milliseconds significantly increased the expression of HLA-A*0201 (*=p<0.05, n=4)

### Example 3

### Induction of MART-1 Positive CTL from HLA-A2 Negative Donors

Monocyte-derived dendritic cells (moDC) from HLA-A*0201-negative donors were transfected with HLA-A*0201 mRNA after maturation. 18-24 hours post-transfection the cells were pulsed with the peptide ELAGIGILTV from the melanoma-associated antigen MART-1 for four hours, before co-culturing them with autologous responder cells (monocyte-depleted PBMC). At d12 of co-culture, the cells were harvested, stained with relevant or control peptide-MHC-pentamer. The percentage of pentamer positive cells was calculated by dividing the number of pentamer positive cells by the number of CD8+ cells. FIG. 3A shows representative data from 2 of 11 donors.

The cells were sorted by flow cytometry (not shown) or by magnetic bead separation with anti-PE microbeads after labeling the pentamer positive cells with PE-conjugated MART-1 pentamers using MACS system from Miltenyi. By running the cells over two subsequent columns, the cells were sorted to very high purity. Representative data from 1 of 3 experiments is shown in FIG. 3B.

The sorted pentamer positive cells were expanded in medium containing PHA, IL-2, IL-15 and allogeneic PBMC as a feeder layer. FIG. 3C shows accumulative expansion for a sorted cell line from one donor over time. Over the course of four weeks, the cells expanded more than 7000 fold (left y-axis, squares). Starting with 0.5×10⁶ cells, this cell line reached an absolute cell count of more than 3.9×10⁹ cells after four weeks (right y-axis, circles). The data are from one of five experiments.

### Example 4

### Production of IFN-Gamma and Mobilization of CD107a and CD107b Upon Antigen-Specific Stimulation

Expanded T-cell lines were harvested and rested for two to three days in medium containing IL-15 only.

Upon stimulation for five hours with autologous EBV-cells that in some cases had been transfected with HLA-A*0201 mRNA and loaded with relevant or irrelevant peptide as indicated in the figure, the cells degranulated and produced IFN-gamma in an antigen-specific, HLA-restricted manner. IFN-gamma production was measured by intracellular cytokine staining. Mobilisation of CD 107 a and b as a marker of degranulation, was measured by adding antibodies to CD107a and CD107b during the stimulation. The cells were analyzed by flow cytometry. Representative data from 1 of 4 experiments is shown in FIG. 4A.

FIG. 4B shows a bar graph summarizing data from four independent donors, showing antigen-specific production of IFN-gamma and degranulation in response to stimulation with autologous, HLA-A*0201-transfected and peptide pulsed EBV-LCL. Controls not -transfected with HLA-A*0201, or loaded with an irrelevant peptide, are also shown. In all experiments, the pentamer positive cells recognized even very low concentrations of the correct peptide (n=4).

Pentamer positive cells were stimulated with T2-cells loaded with decreasing concentration of the relevant (ELAGIGILTV, derived from human melanocyte antigen MART-1) or irrelevant (NLVPMVATV, derived from human cytomegalovirus antigen pp65) A2-binding peptides. The ability of the pentamer positive cells to lyse the peptide loaded target cells was assayed in a standard 4-hour chromium release assay. The results are shown in FIG. 4C. The dotted line represents the background lysis of T2 cells not loaded with any peptide. The cells specifically lysed the target cells loaded with the relevant peptide, even at low peptide concentrations.

### Example 5

### Functional Assays of MART-1 Pentamer Positive CTL from Three Donors

Pentamer positive CTL were rested for 2-3 days in IL-15 before stimulation for 5 hours with autologous EBV-LCL or melanoma tumor cell lines FM-57 and Malme 3M (HLA-A*0201+, MART-1+), A375 (HLA-A*0201+, MART-1−), and Mel 202 (HLA-A*0201−, MART+). Assays using HLA-A*0201 negative cell lines were also carried out with the cells lines transfected with HLA-A*0201 mRNA. The assays were also carried out with cell lines with and without peptide pulsing prior to stimulation. In some assays, HLA-A*0201 positive cell lines were blocked with the anti-HLA class I antibody W6/32. The results are shown in FIGS. 5A, 5B and 5C with the components of the assays indicated. The CTL produced interferon-gamma and degranulated as measured by mobilization of CD107a/b in an antigen-specific, HLA-dependent manner upon stimulation.

### Example 6

### Functional Assay Using CD20₁₈₈₋₁₉₆ Pentamer Positive CTL Lines

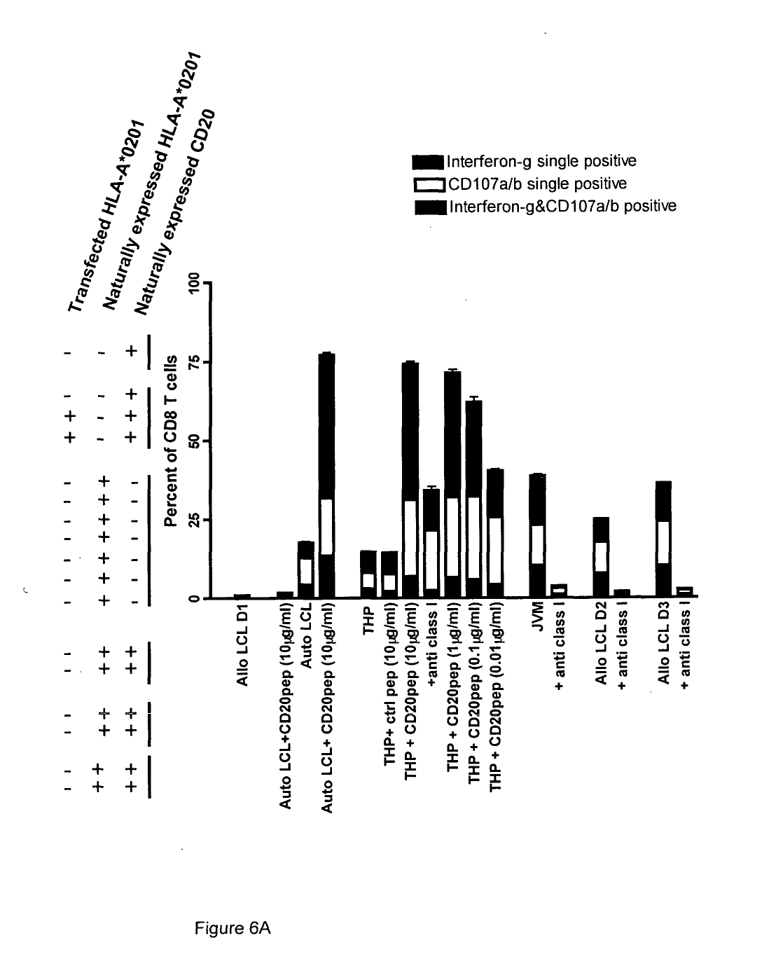

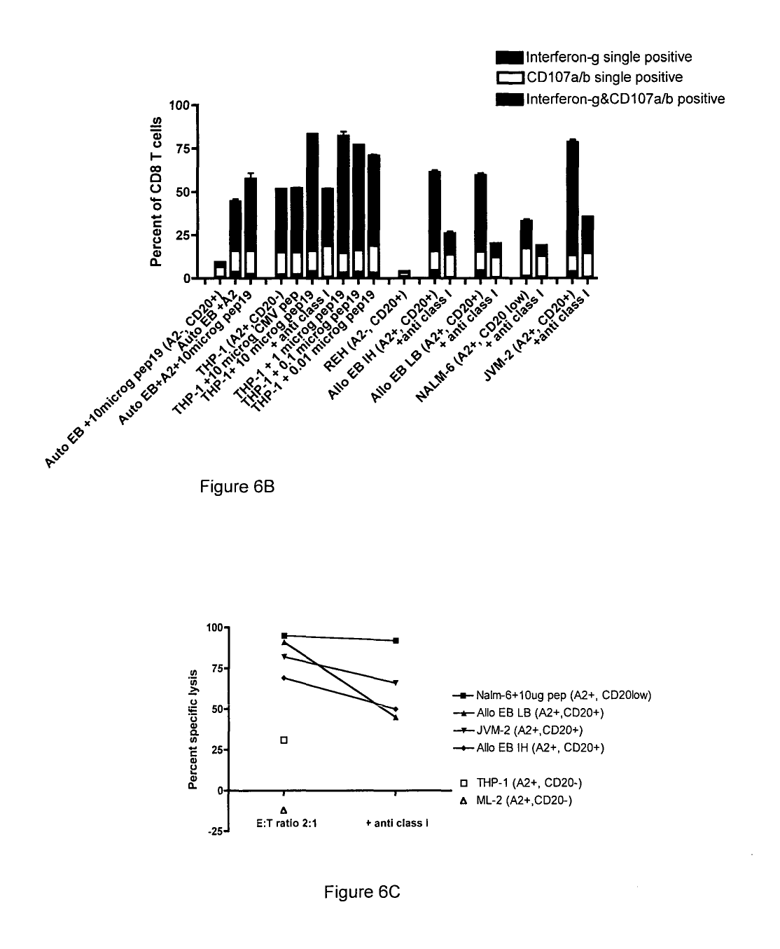

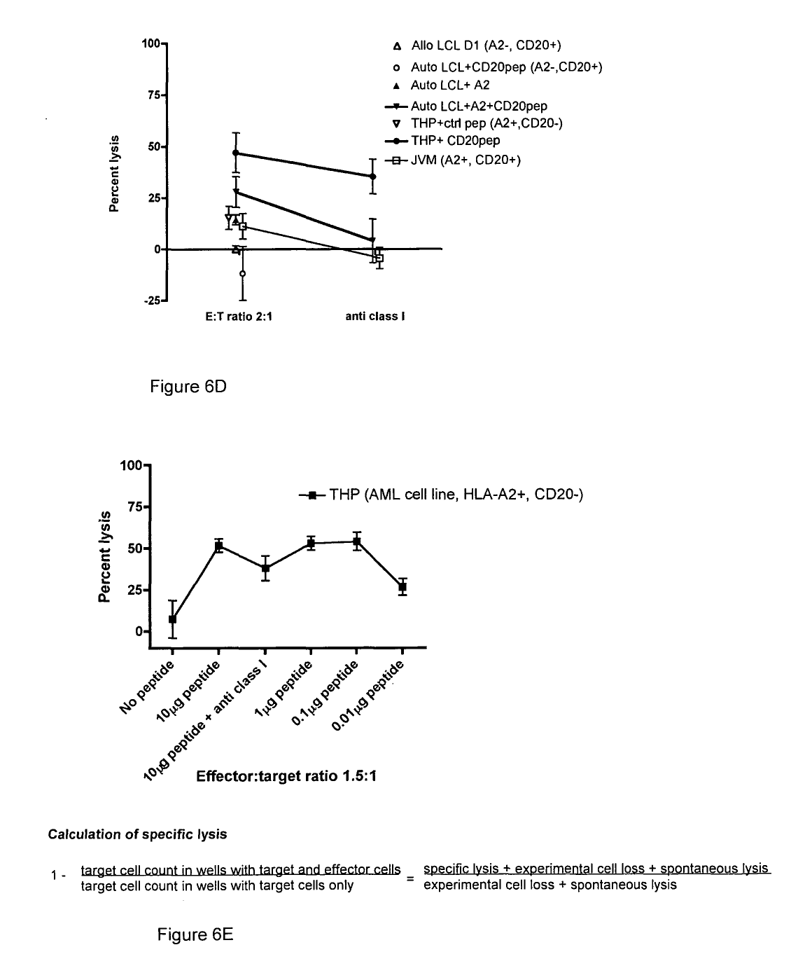

The assay was carried out as described for Example 5. Briefly, T cell lines generated from two different donors against peptide #19 (derived from CD20) were co-cultured with various EBV-LCL or leukemia cell lines for 5 hours and degranulation and interferon gamma production was measured using flow cytometry. The results are shown in FIGS. 6A and 6B and indicate that the allorestricted T cell lines respond vigorously and in a peptide-specific and HLA-A*0201-dependent manner.

A flow cytometry-based cytotoxicity assay was carried out using two CD20₁₈₈₋₁₉₆ pentamer positive CTL lines, generated against peptide #19. Effector cells were co-cultured with various EBV-LCL and leukemia cell lines for 5 hours at an effector:target (E:T) ratio of 2:1 or 1.5:1, as indicated. By adding a fixed amount of beads to all wells, the target cell count in wells with both effector cells and target cells could be compared to the cell count in wells with target cells only. The results are shown in FIGS. 6C and 6D. Specific lysis was calculated by the formula indicated on the figures. The results show that there was a high specific lysis of HLA-A*0201 positive cell lines that express CD20. The graphs are representative of 4 CD20₁₈₈₋₁₉₆ pentamer positive CTL lines. The cell lines indicated on the figure are: The same cytotoxicity assay whose results are shown in FIGS. 6C and 6D was carried out with the target AML cell line THP-1 with and without pulsing with various concentrations of the peptide #19 (CD20 derived) prior to combination with a CTL line generated against the same peptide. The results are shown in FIG. 6E. The graph shows that the killing is peptide specific and that the CTL line has a high affinity for the peptide-HLA-A*0201 complex, as killing is demonstrated even at low peptide concentrations.

JVM-2; a B-prolymphocytic leukemia, THP-1; an acute monocytic leukemia (AML), and EBV-LCL; Epstein-Barr virus transformed B cell lines generated from different donors. Nalm-6; B cell acute lymphoblastic leukemia. REH; B cell acute lymphoblastic leukemia. ML-2; acute myleomonocytic leukemia.

### Example 7

### Selection of Peptides and MHC Binding Assay

Peptides predicted to bind to HLA-A*0201 from the sequences of hematopoietic cell-specific proteins were synthesized and subjected to the REVEAL proprietary MHC molecule binding assay of ProImmune Ltd, UK. The results are shown in Table 1 as a level of binding relative to a pass/fail control binder which is designated 100%.

TABLE 1

| | | % Pass/ | SEQ | |
|---|---|---|---|---|
| Peptide | | fail | ID | |
| ID | Peptide | control | NO. | Protein |
| 1 | YLLDGILFI | 200.79 | 1 | TCR_TV |
| 2 | YLKDGWHMV | 182.19 | 2 | CD5 |
| 3 | ILLWEIFSL | 199.06 | 3 | FLT3 |
| 4 | FLYIGIITI | 136.27 | 4 | CD53 |
| 5 | YLAVGIMFL | 154.63 | 5 | CD64 |
| 6 | SLFEGIYTI | 182.72 | 6 | FLT3 |
| 7 | FLDCQIHRV | 224.04 | 7 | CD127 |
| 8 | FLLGLGLGV | 201.78 | 8 | CD7 |
| 9 | GMDRYLIYV | 169.99 | 9 | CD18 |
| 10 | PLWGGIMYI | 155.04 | 10 | CD20 |
| 11 | SLQSGILTL | 180.55 | 11 | CD127 |
| 12 | FLVNTVLWV | 132.66 | 12 | CD64 |
| 13 | LLDPKLCYL | 204.38 | 13 | TCR_TV |
| 14 | YLLRVIETL | 179.42 | 14 | HA-1 |
| 15 | SLYGPGSYL | 198.44 | 15 | HA-1 |
| 16 | FLPDAFVTM | 163.58 | 16 | TdT |
| 18 | VLDIYGFEV | 161.16 | 17 | HA-2 |
| 19 | SLFLGILSV | 159.78 | 18 | CD20 |
| 20 | SLAQVVMKV | 155.27 | 19 | CD11a |
| 21 | YLMDLSYSM | 198.27 | 20 | CD18 |
| 22 | SLWRRPFNV | 300.83 | 21 | MPO |
| 23 | RLYENRASL | 265.53 | 22 | HA-1 |
| 24 | SMLDDLRNV | 250.54 | 23 | CD18 |
| 25 | ILFAFVSSV | 220.91 | 24 | FLT3 |
| 26 | TLQPPWVSV | 196.66 | 25 | CD64 |
| 27 | LLMSFFILL | 189.14 | 26 | CD53 |
| 28 | LLLPLLLAL | 253.32 | 27 | CD7 |
| 29 | LLSGFFFFA | 210.50 | 28 | CD11a |
| 30 | ALAGVLAIL | 209.87 | 29 | MPO |
| 31 | KLFTSVFGV | 200.14 | 30 | TdT |
| 32 | SLHSETWEI | 179.21 | 31 | CD11a |
| 33 | GLLSPQAQV | 169.6 | 32 | FLT3 |
| 34 | VLQELNVTV | 169.6 | 33 | PR3 |
| 35 | VLIGILLLV | 117.4 | 34 | CD18 |
| 36 | LLLQVSSRV | 180.1 | 35 | CD64 |
| 37 | DMKDSIWWV | 131.3 | 36 | PU.1 |
| 38 | HLYGSNASL | 135.3 | 37 | CD11a |
| 39 | LLRTGGWKV | 124.0 | 38 | CD19 |
| 40 | LMIPAGIYA | 141.9 | 39 | CD20 |
| 41 | GLYQGLSTA | 107.1 | 40 | TCR_TV |
| 42 | LLIHNNFGV | 191.4 | 41 | CD53 |
| 43 | RLFEQVMRI | 191.7 | 42 | MPO |
| 44 | VLTPAQLNV | 175.1 | 43 | MPO |
| 45 | GMGWAAFNL | 192.9 | 44 | CD50 |
| 46 | KMAEAYSEI | 200.0 | 45 | TCR_TV |
| 47 | LLAEVTLAI | 198.6 | 46 | CD53 |
| 48 | ILYPINILI | 213.9 | 47 | CD11a |
| 49 | VLFHNLPSL | 215.9 | 48 | CD53 |
| 50 | YLIFCLCSL | 71.2 | 49 | CD19 |
| 51 | LLLSGAARA | 178.1 | 50 | PR3 |
| 52 | YLRPGQAAA | 125.4 | 51 | CD18 |
| 53 | ALARGLPGA | 215.4 | 52 | CD7 |
| 54 | EMDEELHYA | 117.1 | 53 | CD33 |
| 55 | KLMSPKLYV | 231.8 | 54 | CD19 |
| 56 | SMSEEVTYA | 157.9 | 55 | CLL-1 |
| 57 | NLEEDAYWV | 64.9 | 56 | CD11a |
| 58 | LLLLLIFIV | 6.4 | 57 | CD11a |
| 59 | SICEGTVEV | 130.3 | 58 | CD5 |
| 60 | FIYGVILTA | 149.0 | 59 | TCR_TV |
| 61 | ILTALFLRV | 158.9 | 60 | TCR_TV |
| 62 | FMQPLFLVA | 215.0 | 61 | HA-1 |
| 63 | WLQAQKVQV | 189.8 | 62 | TdT |
| 64 | GLADVAVGA | 206.2 | 63 | CD11a |
| 65 | IMSDSNYVV | 220.3 | 64 | FLT3 |
| 66 | RLYQFLLDL | 233.8 | 65 | PU.1 |
| 67 | KLLLALAGV | 215.9 | 66 | MPO |
| 68 | LLIILLAEV | 99.2 | 67 | CD53 |
| 69 | YISRDTCIV | 132.8 | 68 | HA-2 |
| 70 | FLQDTFPQQ | 49.8 | 69 | CD127 |
| 71 | SMEEAKQLV | 169.4 | 70 | MPO |
| 72 | TLPQLFLKV | 182.3 | 71 | FLT3 |
| 73 | ALASVLLAL | 139.0 | 72 | PR3 |
| 74 | ELLDVSWLI | 220.4 | 73 | TdT |
| 76 | LLEGNLVTL | 172.9 | 74 | CD64 |
| 77 | GLMATPAKL | 218.4 | 75 | MPO |
| 80 | AIWLFIFNV | 199.2 | 76 | CD19 |
| 81 | LLLTISLLV | 250.5 | 77 | CD52 |
| 82 | SVDPRIANV | 295.4 | 78 | MPO |
| 83 | LMGTLIAKV | 315.6 | 79 | HA-1 |
| 84 | LIQPFMFRL | 311.0 | 80 | MPO |
| 85 | LVAPRFLEV | 340.0 | 81 | CD50 |
| 86 | LLWAGALAM | 321.1 | 82 | CD33 |
| 88 | FLLLTISLL | 301.5 | 83 | CD52 |
| 89 | VLLRWEEEL | 184.0 | 84 | CD50 |
| 90 | VLLVVLLVV | 71.5 | 85 | CD5 |
| 91 | YLALGDQML | 197.4 | 86 | CD50 |
| 92 | LLLTNTFGA | 195.7 | 87 | CD11a |
| 93 | YLGYTVTWL | 239.4 | 88 | CD11a |
| 94 | HLLGPWLLL | 211.5 | 89 | CD22 |
| 95 | RMPWYHSSL | 85.6 | 90 | ZAP-70 |
| 96 | KMIMNSLSL | 212.7 | 91 | CD20 |
| 97 | TLWQLVEYL | 198.2 | 92 | ZAP-70 |
| 98 | KLKDLFTEL | 171.3 | 93 | CD11a |
| 99 | GMVFSLLQV | 174.7 | 94 | CD127 |
| 100 | VLWPRACWT | 138.8 | 95 | CD50 |
| 101 | NVAELLHQV | 200.3 | 96 | ZAP-70 |
| 102 | WLSAAPTSL | 207.0 | 97 | CD33 |
| 103 | ELSEDSSNV | 138.9 | 98 | CD18 |
| 104 | LMSFFILLL | 161.4 | 99 | CD53 |
| 105 | VLWHWLLRT | 337.4 | 100 | CD19 |
| 106 | HMRPLAIWL | 56.2 | 101 | CD19 |
| 107 | YLHVALDLL | 278.4 | 102 | MPO |
| 108 | LLFLTPMEV | 282.6 | 103 | CD19 |
| 109 | KIVEWDWKI | 236.4 | 104 | CD48 |
| 110 | VIVGSIIMV | 112.7 | 105 | CD53 |
| 111 | FVANAIIHL | 275.6 | 106 | CD52 |
| 112 | RLLFFLLFL | 42.3 | 107 | CD19 |
| 113 | LLLREHNRL | 195.1 | 108 | MPO |
| 114 | ALAVISFLL | 221.2 | 109 | CD7 |
| 115 | ALYDKTKRI | 259.5 | 110 | TdT_TV2 |
| 116 | WMGGVSEPL | 199.3 | 111 | MPO |
| 117 | CMAGARVQV | 157.7 | 112 | CD50 |
| 118 | KLSLGLPGL | 209.1 | 113 | CD19 |
| 119 | LLFIVVLTL | 113.6 | 114 | FLT3 |
| 120 | VMNGTDPEL | 199.7 | 115 | CD2 |
| 121 | LLMVFVALL | 114.4 | 116 | CD2 |
| 122 | SLTLGNVFV | 150.5 | 117 | CD53 |
| 123 | TVQEGLCVL | 75.2 | 118 | CD33 |
| 124 | SLFAAISGM | 202.6 | 119 | CD20 |
| 125 | VLAEASASQ | 102.6 | 120 | FLT3 |
| 126 | LLQRPGLQL | 230.8 | 121 | CD64 |
| 127 | ALPAALAVI | 263.6 | 122 | CD7 |
| 128 | SMFHVTLKI | 307.4 | 123 | CLL-1 |
| 129 | KLGGDLLRA | 229.4 | 124 | CD18 |
| 130 | ILLLVIWKA | 56.7 | 125 | CD18 |

Modified peptides

| | | | | |
|---|---|---|---|---|
| 131 | FLYGVILTV | 257.0 | 126 | TCR_TV |
| 132 | FLQDTFPQV | 215.9 | 127 | CD127 |
| 133 | FLPDAFVTV | 204.3 | 128 | TdT |
| 134 | YLMDLSYSV | 242.2 | 129 | CD18 |
| 135 | LLQPFMFRV | 329.1 | 130 | MPO |
| 136 | LLIPAGIYV | 275.2 | 131 | CD20 |
| 137 | ELDEELHYV | 217.8 | 132 | CD33 |
| 138 | LLLSGAARV | 210.5 | 133 | PR3 |
| 139 | SVDPRIANV | 231.5 | 134 | MPO |
| 140 | GLYQGLSTV | 213.9 | 135 | TCR_TV |
| 141 | FLQPLFLVV | 270.1 | 136 | HA-1 |
| 142 | LLAPRFLEV | 218.4 | 137 | CD50 |
| 143 | ALARGLPGV | 296.9 | 138 | CD7 |
| 144 | YLRPGQAAV | 227.2 | 139 | CD18 |
| 145 | SLSEEVTYV | 191.2 | 140 | CLL-1 |
| 146 | GLADVAVGV | 213.1 | 141 | CD11a |
| 147 | LLAEVTLAV | 253.8 | 142 | CD53 |
| 148 | LLWAGALAV | 271.9 | 143 | CD33 |
| 149 | ILYPINILV | 320.7 | 144 | CD11a |
| 150 | ELLDVSWLV | 238.6 | 145 | TdT |
| Pass/fail | | 100.0 | | |
| control | | | | |
| Positive | GILGFVFTL | 211.0 | 146 | |
| Control | | | | |

| Peptide ID | Old Peptide | % Pass/fail |
|---|---|---|
| 131 | FIYGVILTA | 149.0 |
| 132 | FLQDTFPQQ | 49.8 |
| 133 | FLPDAFVTM | 163.58 |
| 134 | YLMDLSYSM | 198.27 |
| 135 | LIQPFMFRL | 311.0 |
| 136 | LMIPAGIYA | 141.9 |
| 137 | EMDEELHYA | 117.1 |
| 138 | LLLSGAARA | 178.1 |
| 139 | SVDPRIANV | 295.4 |
| 140 | GLYQGLSTA | 107.1 |
| 141 | FMQPLFLVA | 215.0 |
| 142 | LVAPRFLEV | 340.0 |
| 143 | ALARGLPGA | 215.4 |
| 144 | YLRPGQAAA | 125.4 |
| 145 | SMSEEVTYA | 157.9 |
| 146 | GLADVAVGA | 206.2 |
| 147 | LLAEVTLAI | 198.6 |
| 148 | LLWAGALAM | 321.1 |
| 149 | ILYPINILI | 213.9 |
| 150 | ELLDVSWLI | 220.4 |

MPO = Myeloperoxidase

### Example 8

A selection of peptides from Example 7 were tested in vitro.

Monocyte-derived dendritic cells (moDC) from HLA-A*0201-negative donors were transfected with HLA-A*0201 mRNA after maturation. 18-24 hours post-transfection the cells were pulsed with the peptides indicated in Table 2 for four hours, before co-culturing them with autologous responder cells (monocyte-depleted PBMC). On day 12 and day 19 and in some cases at day 26 of co-culture, the cells were harvested, stained with relevant peptide-MHC-pentamer and analyzed by flow cytometry. The percentage of pentamer positive cells was calculated by dividing the number of pentamer positive cells by the number of CD8+ cells. On day 12 and day 19, the cells were restimulated with autologous EBV-cells that were transfected with HLA-A*0201 mRNA and loaded for four hours with the relevant peptides indicated in Table 2.

The results are shown in Table 2.

TABLE 2

| | Day 12 | | Day 19 | | |
|---|---|---|---|---|---|
| | % pent+/ | | % pent+/ | | |
| Peptide ID | CD8+ | Donors | CD8+ | Donors | Molecule |
| 28 ^{a} | 0.01% | 1/2 | 0.19% | 1/2 | CD7 |
| 55 ^{a} | 0.03% | 1/2 | 0.05% | 1/2 | CD19 |
| 82 ^{a} | 0.04% | 1/2 | 0.19% | 1/2 | MPO |
| 85 ^{a} | 0.13% | 1/4 | 0.30% | 2/5 | CD50 |
| 86 ^{a} | 0.14% | 2/4 | 0.35% | 4/6 | CD33 |
| 93 ^{a} | 0.02% | 1/2 | 0.06% | 2/2 | CD11a |
| 108 ^{a} | 0.03% | 1/2 | 0.09% | 1/2 | CD19 |
| 109 ^{a} | 0.03% | 1/2 | 0.05% | 1/2 | CD48 |
| 126 ^{a} | 0.01% | 1/2 | 0.06% | 1/2 | CD64 |
| 22 ^{b} | 0% | 0/2 | 0.09% | 1/2 | MPO |
| 24 ^{b} | 0% | 0/2 | 0.05% | 1/2 | CD18 |
| 66 ^{b} | 0% | 0/2 | 0.03% | 1/2 | PU.1 |
| 81 ^{b} | 0% | 0/2 | 0.03% | 1/2 | CD52 |
| 88 ^{b} | 0% | 0/2 | 0.08% | 2/3 | CD52 |
| 105 ^{b} | 0% | 0/2 | 0.19% | 4/6 | CD19 |
| 107 ^{b} | 0% | 0/2 | 0.01% | 1/2 | MPO |
| 127 ^{b} | 0% | 0/2 | 0.04% | 1/2 | CD7 |
| 128 ^{c} | | | 0.08% | 1/3 | CLL-1 |
| 7 ^{d} | 0% | 0/2 | 0% | 0/2 | CD127 |
| 25 ^{d} | 0% | 1/2 | 0% | 0/2 | FLT3 |
| 83 ^{d} | 0% | 0/2 | 0% | 0/2 | HA-1 |
| 84 ^{d} | 0% | 0/5 | 0% | 0/4 | MPO |
| 88 ^{d} | 0% | 0/3 | 0% | 0/2 | CD52 |
| 111^{d} | 0% | 0/2 | 0% | 0/2 | CD52 |
| 114 ^{d} | 0% | 0/2 | 0% | 0/2 | CD7 |
| 115 ^{d} | 0% | 0/2 | 0% | 0/2 | TDT_TV2 |
| 129 ^{d} | 0% | 0/2 | 0% | 0/2 | CD18 |

^{a} Peptides giving rise to a positive response in 1 or more donors on day 12 after stimulation (the earliest time point for detection)
^{b} Peptides giving rise to a positive response in 1 or more donors on day 19 after stimulation (second check point after stimulation)
^{c} Results d26. A positive response was observed in 1 donor on day 26, even though there was no detectable response at day 19.

Thus, the peptides marked a, b and c have been shown to be effective in vitro. Those peptides marked d were not shown to be effective in these donors but may be effective in other donors.

### Example 9

In this example, dendritic cells were transfected with mRNA encoding HLA-A*0201, and were found to present efficiently externally loaded peptides from the antigen Melan-A/MART-1 to T cells from HLA-A*0201 negative donors. CD8⁺T cells binding HLA-A*0201/MART-1 pentamers were detected after 12 days of co-culture in all 11 donors. The majority of cells from pentamer⁺ cell lines were CTLs and efficiently killed HLA-A*0201⁺melanoma cells, while sparing HLA-A*0201⁺ B-cells.

Materials and Methods

pCIpA₁₀₂ HLA-A*0201 Construct and In Vitro Transcription of mRNA

Full length HLA-A*0201 cDNA inserted between EcoRI and XhoI sites of a pcDNA3.1 vector (Invitrogen, Groningen, Netherlands) was a gift of Dr. C Britten (Johannes Gutenberg-University of Mainz, Mainz, Germany). A BglII/NotI fragment containing the HLA-A*0201 cDNA together with the CMV and T7 promoters was isolated and inserted between the respective cut sites in the previously described expression vector pCIpA₁₀₂ (Saeboe-Larssen, S., Fossberg, E. and Gaudernack, G., mRNA-based electrotransfection of human dendritic cells and induction of cytotoxic T lymphocyte responses against the telomerase catalytic subunit (hTERT). J Immunol Methods 2002. 259: 191-203) to generate pCIpA₁₀₂ HLA-A*0201. In vitro transcription, extraction, precipitation and storage of mRNA was performed as previously described (Saeboe-Larssen et al. ibid).

Generation of Monocyte-Derived Dendritic Cells

This study was approved by the Regional Ethics Committee and written consent was obtained from subjects in accordance with the Declaration of Helsinki. PBMC isolated from full blood of HLA-A*0201⁻ individuals were incubated for 2 h in CellGro DC medium (CellGenix, Freiburg, Germany) before removal of non-adherent cells that were frozen for subsequent use. Adherent cells were cultured for 6 days in CellGro DC medium supplemented with 500 U/ml IL-4 (Peprotech EC Ltd., London, United Kingdom) and 800 U/ml GM-CSF (Berlex Laboratories, Inc., Richmond, Calif.), followed by 24 h maturation of the cells in the presence of TNF-α (Peprotech EC Ltd.) and lipopolysaccharide from E. coli (LPS, Sigma-Aldrich, St. Louis, Md.) to final concentrations of 2 ng/ml and 10 ng/ml, respectively.

mRNA-Transfection with HLA-A*0201

Harvested moDCs were electroporated in RPMI at 1250 V/cm for 1, 2 or 3 ms with a BTX ECM 830 square wave electroporator (BTX, Harvard Apparatus, Holliston, Mass.). For transfection before maturation, the cells were harvested day 6, transfected and cultured in the presence of LPS and TNF-α for 24 hours before analysis. EBV-transformed lymphoblastoid cell lines (EBV-LCL) were electroporated at 1250 V/cm for 1 ms.

Induction of Allo-Restricted, MART-1 Specific T Cell Lines

Mature moDCs were harvested 18 hours after transfection with HLA-A*0201 mRNA, pulsed with 50 μg/ml MART-1 peptide or CD33₉₋₁₇ peptide for 4 hours, washed and co-cultured with thawed, non-adherent cells (see above) at a ratio of 1:10 in CellGro DC medium in the presence of 10 ng/ml IL-7 (PeproTech EC Ltd.) and 50 pg/ml IL-12 (PeproTech EC Ltd.), with the addition of 10 ng/ml IL-7 on day 7. Cells were harvested day 12 and analyzed for the presence of pentamer⁺ cells. The co-cultures stimulated with the CD33₉₋₁₇ peptide were re-stimulated on day 12 with HLA-A*0201 transfected autologous EBV-LCL for an additional 7 days before harvest.

Sorting Pentamer Positive Cells

Co-cultured cells were harvested and stained with A2/MART-1 pentamer or A2/CD33 pentamer, fluorotag and anti-CD8 according to the manufacturers' instructions. Sorting was performed by flow cytometry (FACS Aria Cell sorter, BD Biosciences, San Jose, Calif.) or by magnetic beads to a purity of >98%. Pentamer⁺ cells labelled with fluorotag conjugated to PE were isolated using MACS Anti-PE MicroBeads (Miltenyi Biotec, Bergisch Gladbach, Germany) followed by separation over two subsequent MACS MS Columns (Miltenyi Biotec).

Antigen-Independent Expansion of Purified, Pentamer Positive Cells

Sorted, pentamer⁺ cells were expanded in the presence of irradiated, allogeneic PBMCs in X-Vivo 20 medium with 5% pooled human serum (HPS), 50 U/ml IL-2, 2 ng/ml IL-15 and 1 μg/ml Phytohaemagglutinin (PHA; Remel, Lenexa, Kans.). The ⁻cells were re-stimulated weekly and split and/or supplemented with fresh medium containing IL-2, IL-15 and PHA, when needed.

Antibodies and Flow Cytometry

The following antibodies were used: CD3 FITC, CD3 APC, CD3 PerCP-Cy5.5, CD8 FITC, CD8 APC, CD8 PerCP-Cy5.5, CD80 PE, CD86 FITC, HLA-DR PerCP, IFN-γ PE, CD107a FITC, CD107b FITC (all from BD Biosciences), HLA-A*0201 PE (ProImmune Ltd.), CD80 Alexa 647, CD83 Alexa 488 and IFN-γ FITC (Serotec Ltd, Oxford, UK). The antibodies to HLA-A*0201, CD107a and CD107b were also conjugated to Alexa 647 (Molecular Probes Inc., Eugene, Oreg.) by the inventors. The Vβchains were stained with the Beta Mark Kit and Vβ 3 6.7 FITC (Beckman-Coulter, Fullerton, Calif.) (nomenclature according to Arden, B., Clark, S. P., Kabelitz, D. and Mak, T. W., Human T-cell receptor variable gene segment families. Immunogenetics 1995. 42: 455-500.) Cells were analyzed on FACS Calibur and LSRII flow cytometers (BD Biosciences), and data analysis performed with CellQuest Pro, FACS DiVa (BD Biosciences) and FlowJo (Tree Star Inc., Ashland, Oreg.) software.

The pentamer⁺ T cells were tested for the ability to produce interferon-γ and lyse target cells using a modification of previously described assays for flow cytometry (Openshaw, P., Murphy, E. E., Hosken, N. A., Maino, V., Davis, K., Murphy, K. and O'Garra, A., Heterogeneity of intracellular cytokine synthesis at the single-cell level in polarized T helper 1 and T helper 2 populations. J Exp Med 1995. 182: 1357-1367. Betts, M. R., Brenchley, J. M., Price, D. A., De Rosa, S. C., Douek, D. C., Roederer, M. and Koup, R. A., Sensitive and viable identification of antigen-specific CD8+ T cells by a flow cytometric assay for degranulation. J Immunol Methods 2003. 281: 65-78. Betts, M. R., Nason, M. C., West, S. M., De Rosa, S. C., Migueles, S. A., Abraham, J., Lederman, M. M., Benito, J. M., Goepfert, P. A., Connors, M., Roederer, M. and Koup, R. A., HIV nonprogressors preferentially maintain highly functional HIV-specific CD8+ T cells. Blood 2006. 107: 4781-4789.). Briefly, effector and target cells were washed and combined in 96-well plates in X-Vivo 20 at a ratio of 2:1 in the presence of anti-CD107a and anti-CD107b (each 0.25 μg/ml), 10 μg/ml Brefeldin A (Sigma-Aldrich) and 10 nM Monensin A (Sigma-Aldrich), and 50 anti-HLA class I antibody (clone W6/32, a gift from the Lnstitute of Immunology, Rikshospitalet, Oslo) where indicated. Following a 5 h incubation, cells were stained as previously described (Openshaw, P., Murphy, E. E., Hosken, N. A., Maino, V., Davis, K., Murphy, K. and O'Garra, A., Heterogeneity of intracellular cytokine synthesis at the single-cell level in polarized T helper 1 and T helper 2 populations. J Exp Med 1995. 182: 1357-1367). For measurement of cell lysis, the cells were incubated for 5 h in X-Vivo 20 at effector:target ratios of 2:1 in 96-well plates, harvested and stained for surface markers, Annexin V FITC (BD Biosciences) and Propidium iodide (Sigma-Aldrich), according to the manufacturer's protocols. A fixed number of fluorescent polymer beads were added to each sample to allow calculation of the absolute number of remaining, live target cells in each sample. Cells within the live scatter gate, and staining negatively for Annexin V and Propidium iodide, were considered live. Each assay was performed in triplicate. The following formula was used to calculate lysis: % specific lysis=1−(Number of live target cells in wells with responders/Number of live targets in control wells).

⁵¹Cr-Release Assay

A standard ⁵¹Cr-release assay was performed as previously described (Jedema, I., van der Werff, N. M., Barge, R. M., Willemze, R. and Falkenburg, J. H., New CFSE-based assay to determine susceptibility to lysis by cytotoxic T cells of leukemic precursor cells within a heterogeneous target cell population. Blood 2004. 103: 2677-2682.).

Results

Transfection of Monocyte-Derived Dendritic Cells with MA-A*0201

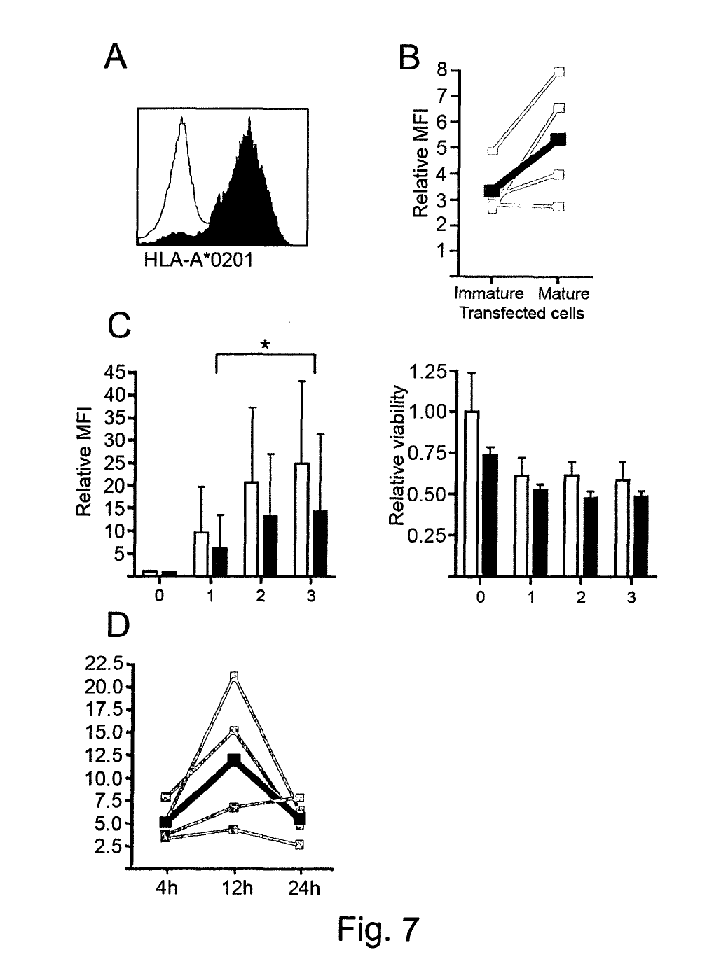

MoDCs from HLA-A*0201⁻ individuals were electroporated in the presence of mRNA encoding HLA-A*0201. Transfection was highly efficient, as >90% of the surviving cells expressed the allogeneic HLA molecule (A2-moDC) at all conditions tested (FIG. 7A and not shown). In three of four experiments, the highest levels of expression were observed when transfection was performed after DC maturation (FIG. 7B). Increasing the pulse length from one to three milliseconds also enhanced expression (FIG. 7C, left diagram). The viability was about 60% after the first 24 h, independently of pulse length. Between 24 and 48 h, cell loss of A2-moDC was similar to that of non-transfected cells (FIG. 7C, right diagram). Expression of HLA-A*0201 was detected at 4 h, increased from 4 to 12 h, decreased again at 24 h (FIG. 7E), but was clearly retained also at 48 h (FIG. 7C, right diagram). As shown below, this time window was sufficient to induce T cell activation.

HLA-A*0201 Transfected MoDC Loaded with MART-1 Peptide Rapidly Induce MART-1-Specific Allo-Restricted CD8⁺ T Cells.

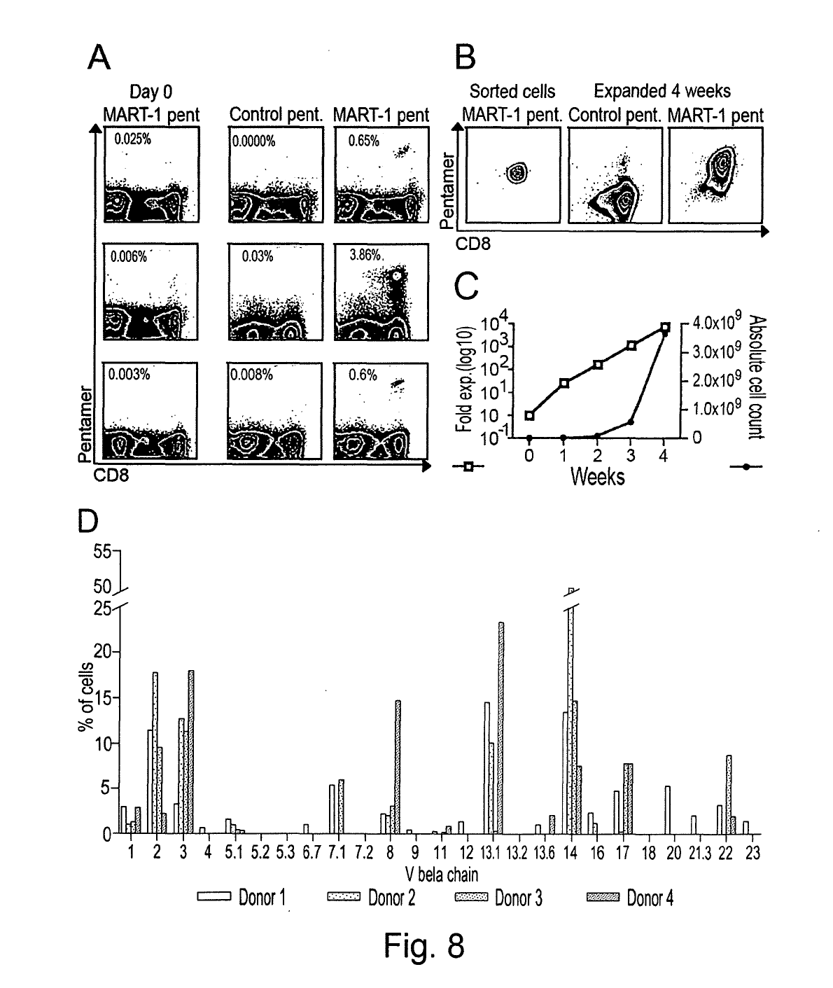

Previous studies have shown that HLA-A*0201 negative individuals have detectable levels of CD8⁺ T cells reactive with HLA-A*0201-MelA₍₂₆₋₃₅₎ (MART-1) tetramers. In the present study, the median frequency of A2/MART-1 pentamer⁺ cells was measured to 0.006% of CD3⁺CD8⁺ cells (range 0.002-0.047%, n=12). This frequency was not significantly different from that of cells reactive with a control pentamer incorporating a CMV-derived peptide (median 0.004%, range 0.000-0.067%). In contrast, HLA-A*0201⁻MART-1⁺ cells were observed in 11/11 donors after 12 days of co-culture with A2-moDC loaded with MART-1 peptide. The pentamer⁺ T cells were detected as a discrete positive population of brightly staining cells with a median frequency of 0.36% of CD8⁺ cells, (range 0.02-3.86% n=11, and FIG. 8A). Pentamer binding was clearly peptide-dependent, since the frequency of cells reactive with the control pentamer did not increase after the culture period (FIG. 8A).

Expansion of Polyclonal, MART-1 Pentamer Positive T Cell Lines

A2/MART-1 pentamer⁺ T cells were sorted on day 12 by FACS or by magnetic beads to a purity >98% (FIG. 8B and further results, not shown). Sorted, MART-1 pentamer⁺ cells were expanded independently of antigen for four weeks. The cells remained pentamer⁺ with a high purity (FIG. 8B). During this period, the cells could be expanded 7318-fold, as shown in FIG. 8C. Starting with 0.5×10⁶ pentamer⁺ cells, this would yield more than 3.9×10⁹ cells after 4 weeks. A2/MART-1 pentamer⁺ T cell lines were labelled with antibodies specific for a panel of TCR Vβ chains to assess whether the expanded cells were polyclonal (FIG. 8D). The results showed that Vβ chains 1, 2, 3, 5.1, 8, 13.1, 14 and 17 were used by all 4 lines, whereas cells positive for Vβ 11 and 22 were found in three lines. Vβ5.2, 5.3, 7.2, 13.2 and 18 were not detected in any of the cell lines. The cumulated frequencies of cells staining positively with these antibodies in each of the 4 cell lines were 97%, 83%, 80% and 64%, respectively. A similar panel of anti-Vα chains is currently not available. However, since previous studies have shown the frequent use of Vα 2.1/TRAV12-2 for A2/MART-1 pentamer⁺ T cells in HLA-A2⁺ and HLA-A2⁻ individuals, we generated T cell clones from the pentamer⁺ cells, that were tested for this particular Vα chain by PCR. The results showed that 65% (15/23) of the clones were positive. The frequency of Vα 2.1/TRAV12-2 among CD8⁺ T cells from human blood averages 10% (Moss, P. A., Rosenberg, W. M. and Bell, J. I., The human T cell receptor in health and disease. Annu Rev Immunol 1992. 10: 71-96.). Thus, the preferential Vα usage of MART-reactive T cells shown in previous studies was confirmed.

Pentamer Positive T Cells Recognize Peptide-Pulsed Target Cells in an Antigen-Specific, HLA-Restricted Manner

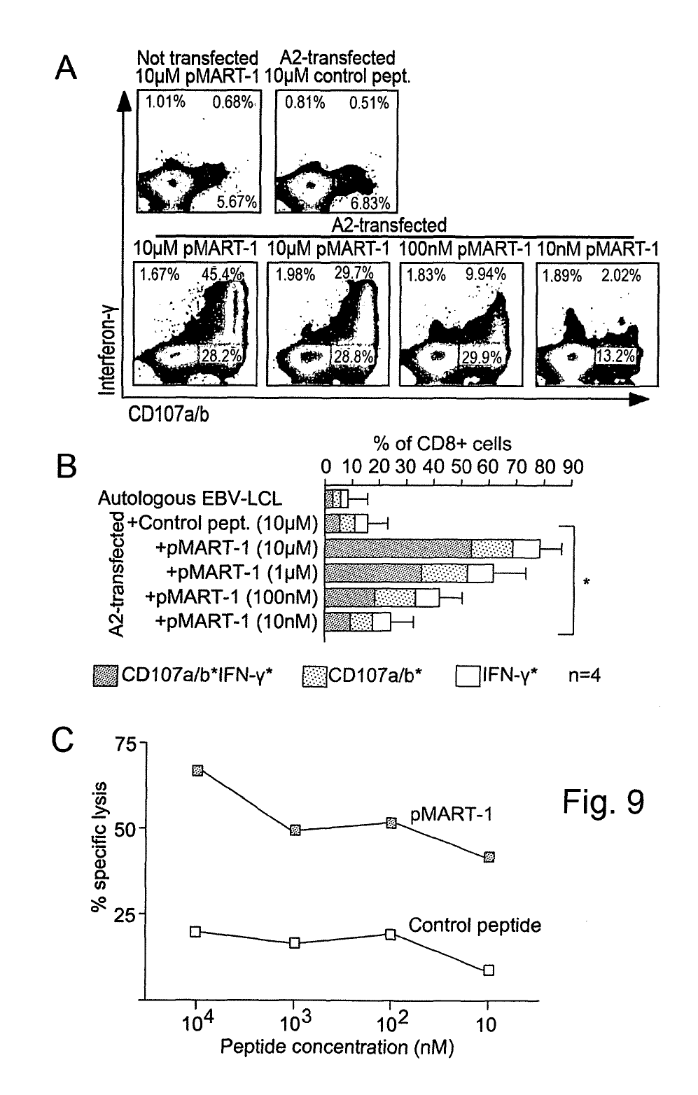

As an initial test for antigen-specificity, allo-restricted T cell lines were incubated with EBV-LCL that were negative or positive for HLA-A*0201 and loaded with the MART-1 peptide. T cell activation was detected at the single cell level by flow cytometric measurement of interferon-γ production and translocation of the cytotoxic granule-associated protein CD107a/b to the cell surface. The results in FIGS. 9A and B show that the large majority of the cells in each of 4 cell lines were activated when both HLA-A*0201 and the MART-1 peptide was present on the target cells. Moreover, there was a clear dose-dependent relationship between the amount of peptide added and T cell activation. It should be noted that no attempts were made to deplete surface-bound peptides on EBV-LCL prior to peptide loading, and that the cells were washed before incubation with the T lymphocytes. The concentrations of peptides needed for responses are therefore expected to be higher than those required for cytotoxicity assays of e.g. T2 cells. The results were confirmed using a standard ⁵¹Cr-release assay (FIG. 9C). The T cell lines efficiently killed MART-1 peptide-pulsed T2-cells, whereas low levels of killing were found where pulsed with the control peptide NLVPMVATV (SEQ ID NO. 147), at an effector:target ratio of 10:1. Collectively, these results show that the allo-restricted cell lines generated in the presence of A2-moDC, contain high frequencies of CTLs with high antigen-specificity.

MART-1 Apecific, Allo-Restricted T Cells Kill HLA-A*0201 Positive Melanoma Cells, Whereas HLA-A*0201 Positive EBV-LCL are Spared Unless Loaded with the MART-1 Peptide

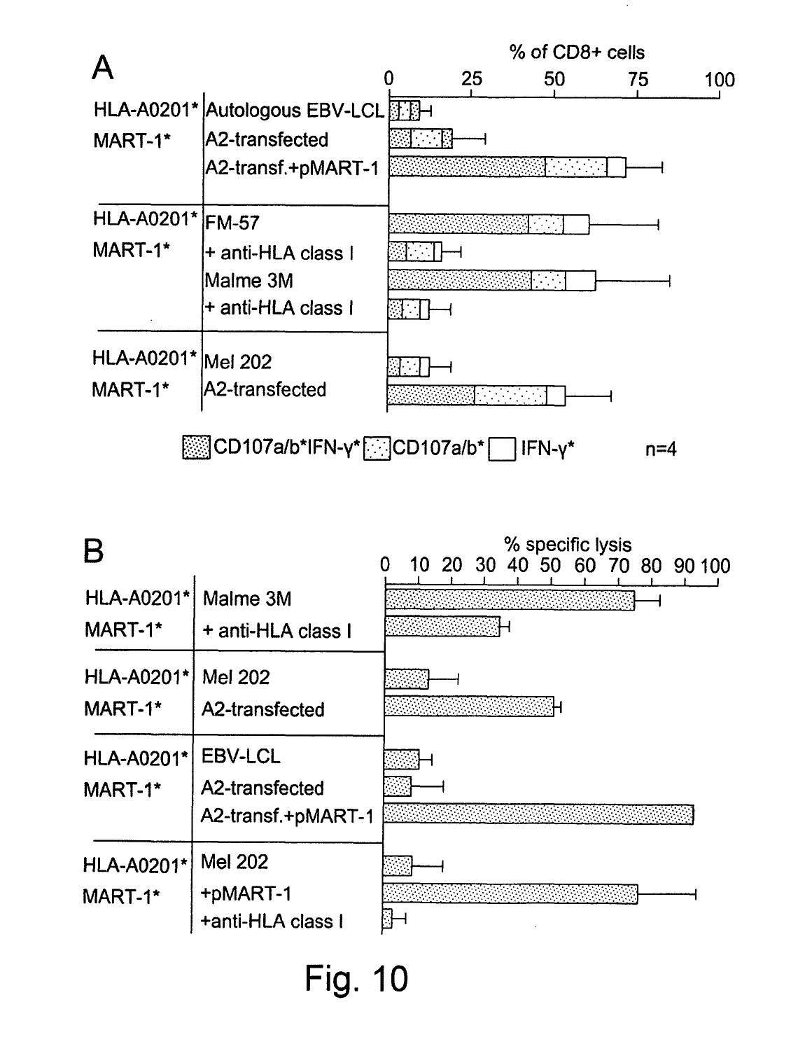

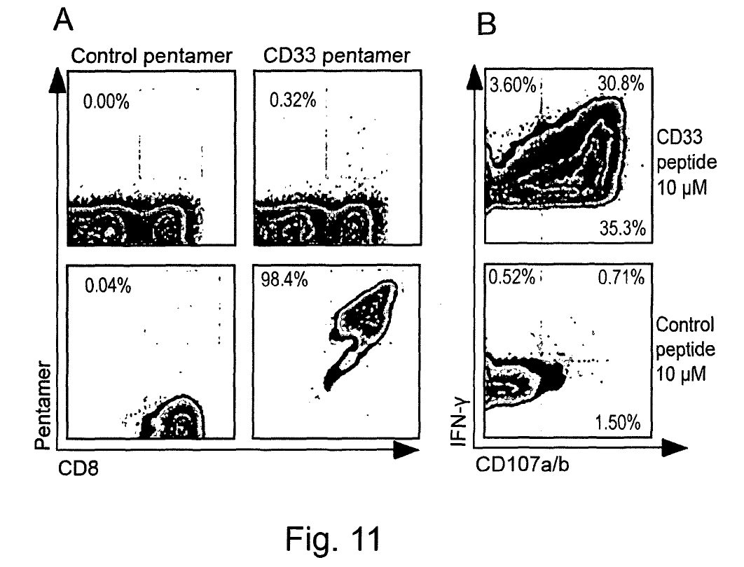

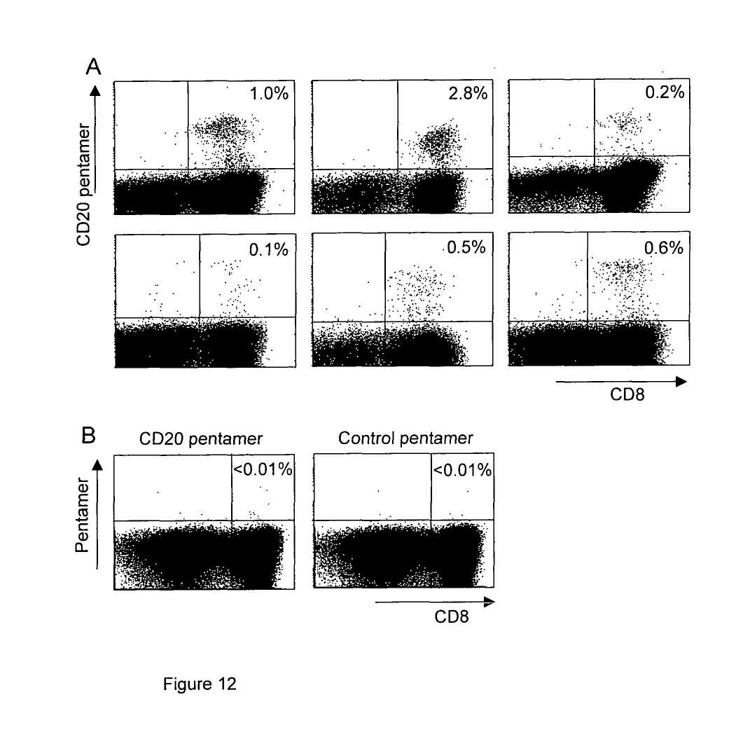

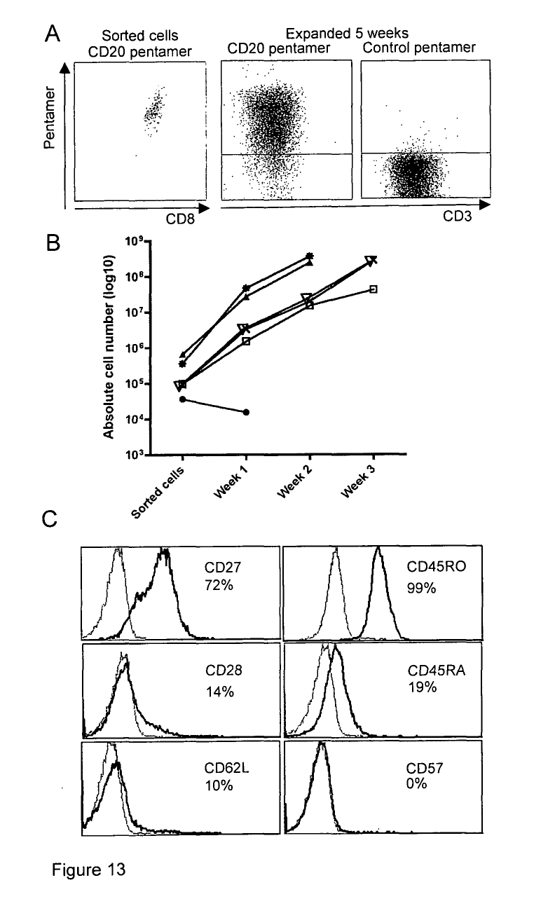

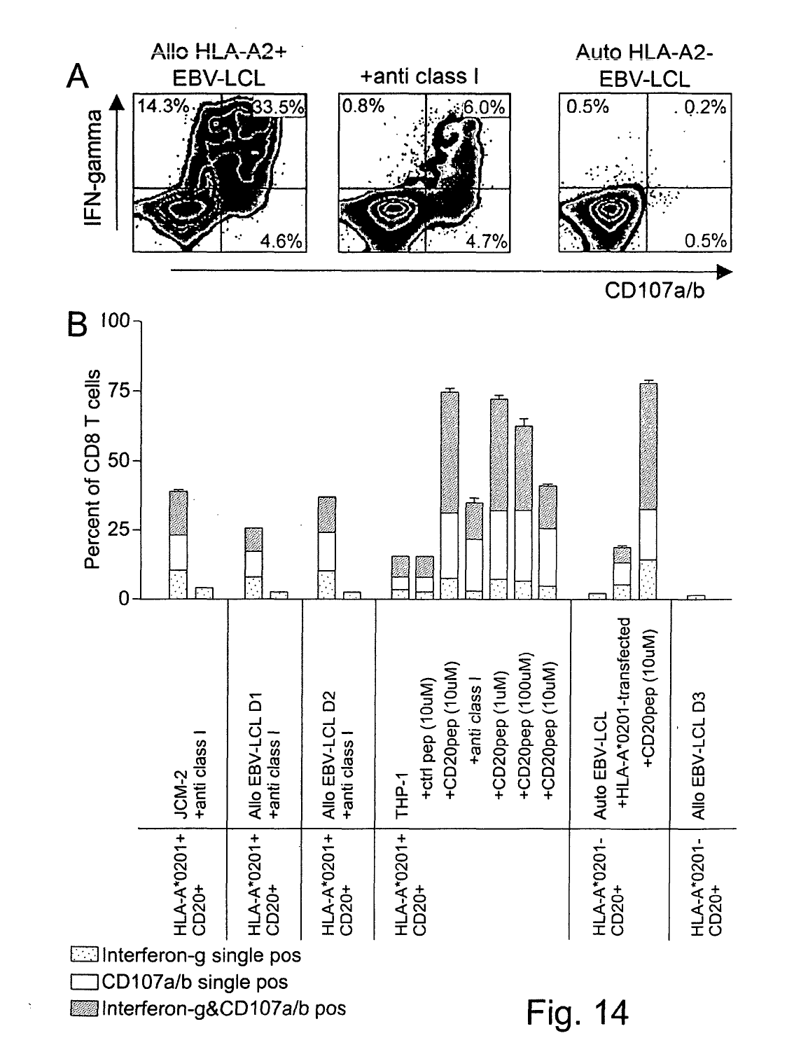

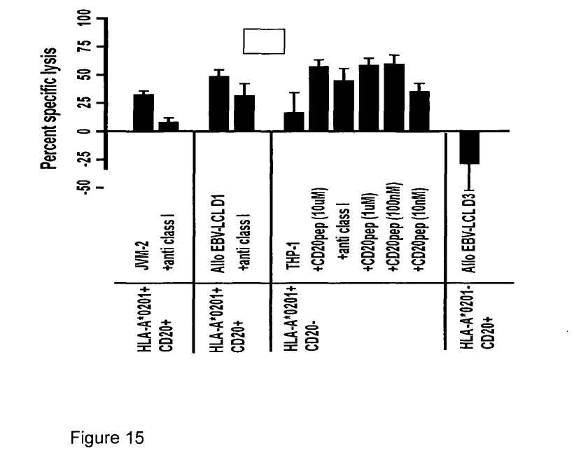

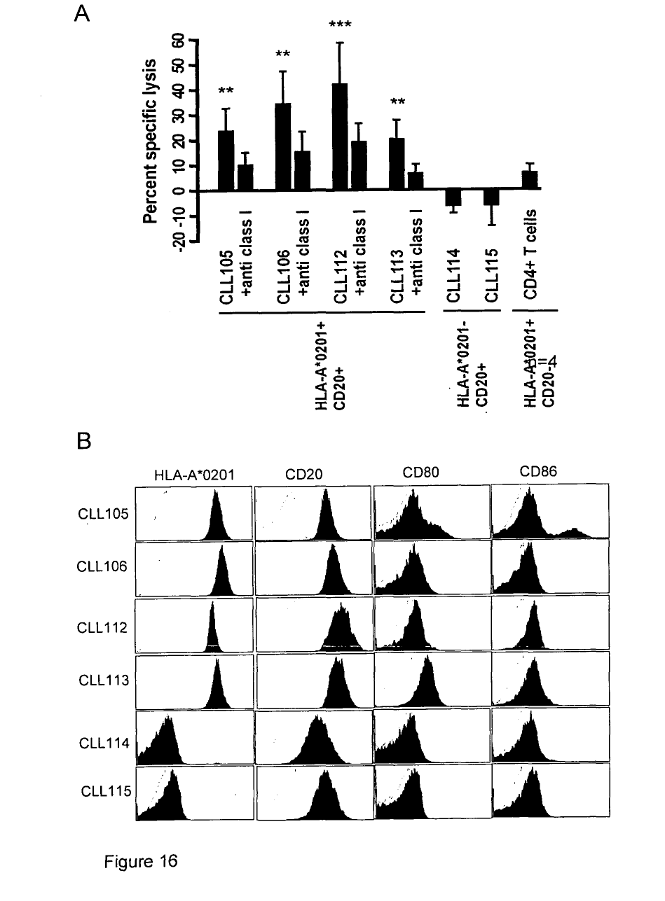

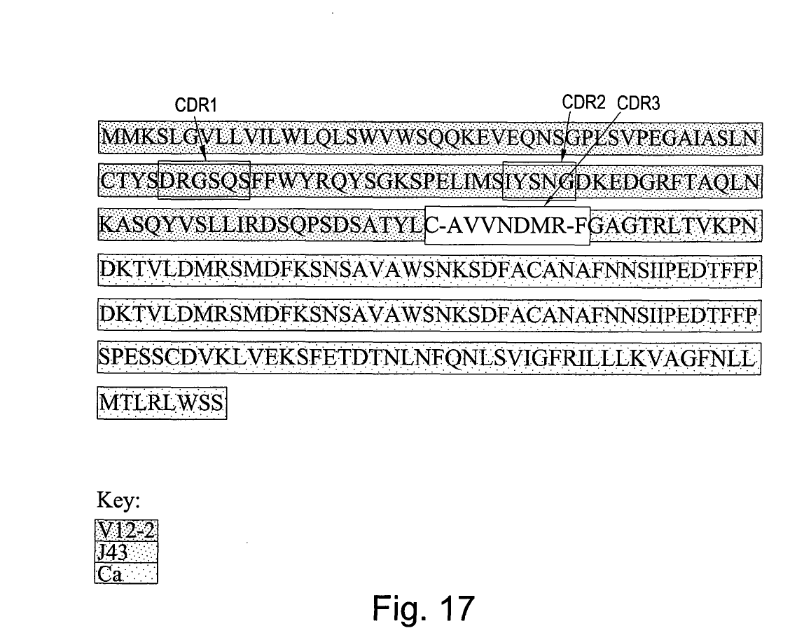

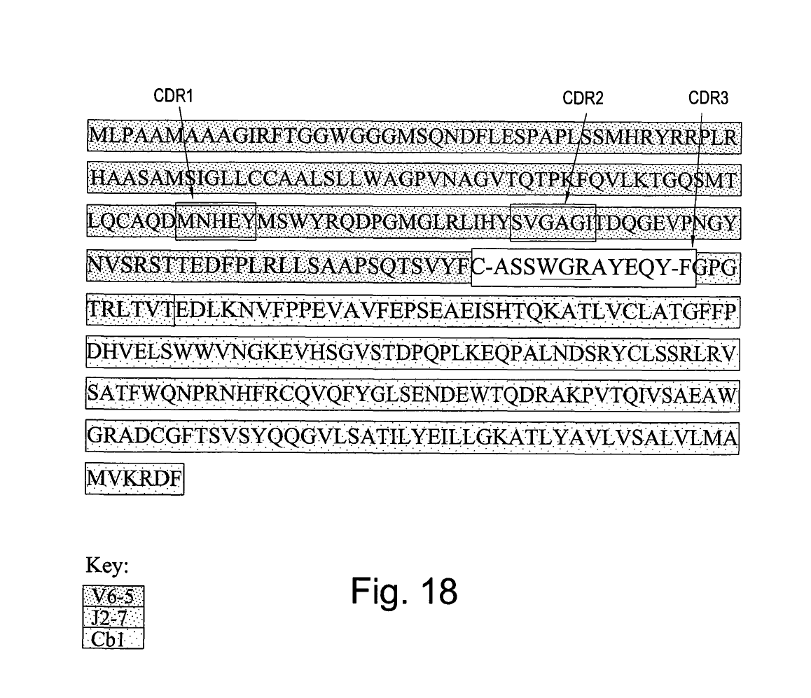

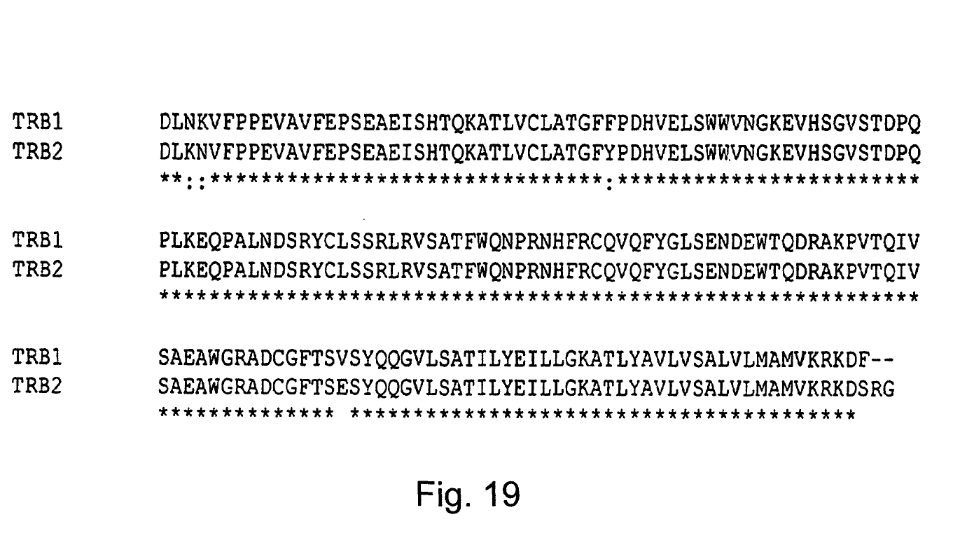

Killing of tumor cells with endogenous expression of antigen is critical for the therapeutic benefit of adoptive T cell transfer. The results in FIG. 10A show that the majority of allo-restricted MART-1 specific T cells obtained from four different donors were activated by melanoma cells lines (FM-57 and Malme 3M) expressing HLA-A*0201 and MART-1. Cytokine production and release of cytotoxic granules was almost completely blocked by anti-HLA class I. A third MART-1-positive cell line, lacking HLA-A*0201 (Mel202), activated the cells only after transfection with HLA-A*0201. Antigen-specificity was further documented by results showing that HLA-A*0201⁺ EBV-LCL selectively induced activation when pulsed with the MART-1 peptide (FIG. 10A).

To verify that allo-restricted CTLs were capable of killing defined cell types, tumor cell lysis was analyzed directly by flow cytometric measurement of remaining, viable tumor cells in CTL co-cultures. Malme 3M cells (HLA-A*0201⁺ MART-1⁺) cells were efficiently lysed at low E:T ratios of 2:1, and killing was inhibited by anti-HLA class I antibody (FIG. 10B). Similar results were obtained with the HLA-A*0201⁺ MART-1⁺ melanoma cell line FM-57 (data not shown). Mel202 cells (HLA-A*0201⁻) were killed only when transfected with the alloantigen. In contrast, lysis of allogeneic EBV-LCL was only observed when HLA-A*0201 was expressed and after MART-1 peptide loading (FIG. 10B). Collectively, these results demonstrate that A2-moDC can be used to select allo-restricted CTLs capable of efficient killing of defined tumor cells.

Allo-Resticted, Antigen-Specific CTL Lines could be Generated Against other Self-Peptides

To demonstrate that the results above were not due to unique immunogenic properties of MART-1, we tested a peptide derived from the CD33 antigen expressed on myeloid cells. The amino acid sequence 9-17 was identified as an HLA-A*0201-binding candidate by a computer algorithm (Buus, S., Lauemoller, S. L., Worning, P., Kesmir, C., Frimurer, T., Corbet, S., Fomsgaard, A., Hilden, J., Holm, A. and Brunak, S., Sensitive quantitative predictions of peptide-MHC binding by a ‘Query by Committee’ artificial neural network approach. Tissue Antigens 2003. 62: 378-384.). The frequencies of pentamer⁺ cells obtained with this peptide were 0.13%, 0.32% and 0.55% in three donors, respectively. The response to CD33₉₋₁₇ was detected after 19 days of culture (FIG. 11A). Sorted cells remained pentamer⁺ after 19 additional days of expansion. The majority of the cells in the line produced IFN-γ and released cytotoxic granules when stimulated with CD33 peptide-loaded HLA-A*0201-transfected EBV-LCL, but not with a control peptide (FIG. 11B). These results indicate that the approach presented here allows highly peptide-specific, allo-restricted CTLs to be generated against multiple self-peptides. To find out if the CD33₉₋₁₇ is naturally presented by cells that express CD33, would however require further testing.

Discussion

The results in this example show that DC transfected with a foreign HLA molecule efficiently present externally loaded peptides and expand allo-restricted CTLs. The CTL-lines induced by DC were highly specific for the cognate antigen and efficiently killed melanoma cells expressing the antigen while sparing HLA-A*0201⁺ B cells.

Previously, cells with defects in the antigen-processing pathway, such as T2 cells (DeMars, R., Chang, C. C., Shaw, S., Reitnauer, P. J. and Sondel, P. M., Homozygous deletions that simultaneously eliminate expressions of class I and class II antigens of EBV-transformed B-lymphoblastoid cells. I. Reduced proliferative responses of autologous and allogeneic T cells to mutant cells that have decreased expression of class II antigens. Hum Immunol 1984. 11: 77-97) and RMA-S cells (Townsend, A., Ohlen, C., Foster, L., Bastin, J., Ljunggren, H. G. and Karre, K., A mutant cell in which association of class I heavy and light chains is induced by viral peptides. Cold Spring Harb Symp Quant Biol 1989. 54 Pt 1: 299-308.), have been widely used to induce allo-restricted T cells. Both lines express low levels of HLA class I, and present a limited range of endogenous peptides. Externally loaded peptides displace endogenous peptides and stabilize surface HLA molecules (Alexander, J., Payne, J. A., Murray, R., Frelinger, J. A. and Cresswell, P., Differential transport requirements of HLA and H-2 class I glycoproteins. Immunogenetics 1989. 29: 380-388.). The protocol minimizes presentation of non-relevant HLA-peptide complexes that might compete with the desired antigen. However, the cells are not professional antigen presenting cells. An important finding in the present example is that irrelevant allo-reactivity did not prevent effective expansion of rare allo-restricted T cells. A2/MART-1 pentamer⁺ cells were readily identified among cells from all donors following only 12 days of co-culture with the transfected DCs. This was not due to high frequencies of MART-1 reactive cells in HLA-A0201⁻ donors, which were similar to those of cells staining with the control pentamer.

The example demonstrates the successful and rapid generation of antigen-specific CTLs efficiently killing tumor cells.

The specificity of allo-restricted T cells has been much debated (Felix, N. J. and Allen, P. M., Specificity of T-cell alloreactivity. Nat Rev Immunol 2007. 7: 942-953.). Importantly, the allo-restricted CTL lines generated in this example, were not cytotoxic to HLA-A*0201⁺ EBV-LCL. These cells express a large variety of HLA-A*0201-peptide complexes on their surface compared with e.g. T2 cells loaded with a single peptide. This indicates that the allo-restricted CTLs were highly peptide-restricted and therefore good candidates for adoptive transfer.

In summary, the approach presented in this example allows the rapid and reproducible generation of CTL lines from healthy HLA-A*0201 negative donors with the ability to efficiently and specifically kill melanoma target cells. The cells can be expanded to numbers sufficient for use in adoptive transfer.

### Example 10

In this example CD8+ cytotoxic T cells (CTL) were generated that efficiently killed primary B chronic lymphoid leukaemia (CLL) cells while sparing non-B cells. The CTLs were raised against a peptide (SEQ ID NO. 18) from the B cell-specific antigen CD20 in complex with HLA-A*0201. Since HLA-A*0201 positive donors are tolerant to this self-antigen, T cells from HLA-A*0201 negative individuals were exposed to autologous dendritic cells engineered to present HLA-A*0201/SEQ ID NO. 18 complexes. The approach led to successful generation of HLA-A*0201/SEQ ID NO. 18-reactive T cells in all 6 donors in only 19 days.

Patients, Materials and Methods

Patients, Healthy Donors and Cell Lines

This study was approved by the Regional Ethics Committee. In accordance with the declaration of Helsinki, patients with chronic lymphocytic leukemia (CLL) and healthy donors donated blood after written consent. PBMCs from CLL patients were cryopreserved and stored in liquid nitrogen. Karyotype and V-heavy chain mutational status was determined by standard methods. (Koller, Bekele et al. 2006)

The following leukemia cell lines were obtained from Deutsche Sammlung von Mikroorganismen and Zellkulturen: NALM-6 (HLA-A*0201⁺, CD20^{low}) (Flavell, Warnes et al. 2006), THP-1 (HLA-A*0201⁺, CD20⁻) and JVM-2 (HLA-A*0201⁺, CD20⁺). Epstein-Barr virus-lymphoblastoid cell lines (EBV-LCL) were generated using standard procedures. All cell lines were maintained in RPMI 1640 (Gibco, Invitrogen, Paisley, UK) containing penicillin/streptomycin (50 U/ml/50 μg/ml, Sigma-Aldrich, St Louis, Mo.) and 10% fetal calf-serum (Gibco).

Peptides and Pentamers

The peptides SLFLGILSV (CD20₁₈₈₋₁₉₆, referred to as SEQ ID NO. 18, or, in this example as “CD20”), first described in (Bae, Martinson et al. 2005), and NLVPMVATV (SEQ ID NO. 147) (human cytomegalovirus pp65₄₉₅₋₅₀₃, used as control peptide) and corresponding HLA-A*0201 pentamers (referred to as A2/CD20 pentamer and control pentamer, respectively) were synthesized by Proimmune Ltd (Oxford, UK). Pentamers were labelled with fluorotags conjugated to APC or PE, as recommended by the manufacturer.

pCIpA₁₀₂ HLA-A*0201 Construct and In Vitro Transcription of mRNA

Details were as per Example 9 save that for in vitro transcription of mRNA, the construct was linearized with MfeI and purified by using Wizard SV Gel and PCR Clean-Up System (Promega, Madison, Wis.) as described in the kit manual.

Generation of Monocyte-Derived Dendritic Cells

Materials and methods were as per Example 9.

mRNA-Transfection with HLA-A*0201

Harvested moDCs were electroporated in RPMI at 1250 V/cm for 3 ms with a BTX ECM 830 square wave electroporator (BTX, Harvard Apparatus, Holliston, Mass.). EBV-transformed lymphoblastoid cell lines (EBV-LCL) were electroporated at 1250 V/cm for 1 ms.

Induction of T Cell Lines

Mature moDCs were harvested 18 hours after transfection with HLA-A*0201 mRNA (HLA-A*0201 negative donors), or left untransfected (HLA-A*0201 positive donors), pulsed with 50 μg/ml peptide for 4 hours, irradiated (25Gy), washed and co-cultured with thawed, autologous, non-adherent cells at a ratio of 1:10 in CeliGro DC medium in the presence of 10 ng/ml IL-7 (PeproTech EC Ltd.), 50 pg/ml IL-12 (PeproTech EC Ltd.) and 1 μg/ml purified protein derivative-1 (gift from Prof. L. Sollid's lab, Rikshospitalet, Oslo) with the addition of 10 ng/ml IL-7 on day 7. Cells were analyzed for the presence of pentamer⁺ cells, harvested and re-stimulated on day 12 with HLA-A*0201-transfected, peptide-pulsed autologous EBV-LCL. Cells were co-cultured in X-vivo 20 (Lonza, Verviers, Belgium) with 5% pooled human serum, 10 U/ml IL-2 (R&D systems, Minneapolis, Minn.) was added on day 14 and the cells harvested for cell sorting day 19.

Sorting Pentamer Positive Cells

Harvested cells were stained with A2/CD20 pentamer, fluorotag and anti-CD8 according to the manufacturers' instructions. Sorting was performed by flow cytometry (FACS Aria Cell sorter, BD Biosciences, San Jose, Calif.) or by magnetic beads to a purity of >98%. Pentamer⁺ cells labelled with fluorotag conjugated to PE were isolated using MACS anti-PE MicroBeads (Miltenyi Biotec, Bergisch Gladbach, Germany) followed by separation over two subsequent MACS MS Columns (Miltenyi Biotec).

Antigen-Independent Expansion of Purified, Pentamer Positive Cells

Materials and methods were as per Example 9.

Antibodies and Flow Cytometry

The following antibodies were used: CD3 FITC, CD3 APC, CD5 PerCP-Cy5.5, CD8 FITC, CD8 PE, CD8 APC, CD8 PerCP-Cy5.5, CD19 APC, CD20 PE, CD27 FITC, CD28 PE, CD45RA PE, CD45RO FITC, CD57 FITC, CD62L PE, CD80 PE, CD86 FITC, IFN-γ PE (all from BD Biosciences). The antibodies to HLA-A*0201 (Serotec Ltd, Oxford, UK), CD107a and CD107b (BD Biosciences) were conjugated to fluorescein and/or Alexa 647 (Molecular Probes Inc., Eugene, Oreg.) by the authors. Cells were analyzed on FACS Calibur and Facs Canto II cytometers (BD Biosciences), and data analysis performed with CellQuest Pro, FACS DiVa (BD Biosciences) and FlowJo (Tree Star Inc., Ashland, Oreg.) softwares.

The pentamer⁺ T cells were tested for the ability to produce interferon-γ and lyse target cells using a modification of previously described assays for flow cytometry (Openshaw, Murphy et al. 1995; Betts, Brenchley et al. 2003; Betts, Nason et al. 2006). Briefly, effector and target cells were washed and combined in 96-well plates in X-Vivo 20 at a ratio of 2:1 in the presence of anti-CD107a and anti-CD107b (each 0.25 μg/ml), 10 μg/ml Brefeldin A (Sigma-Aldrich) and 10 nM Monensin A (Sigma-Aldrich), and 50 μg/ml anti-HLA class I antibody (clone W6/32, a gift from the Institute of Immunology, Rikshospitalet, Oslo), where indicated. Following a 5 h incubation, cells were stained as previously described. (Openshaw, Murphy et al. 1995) For measurement of lysis of primary CLL cells, the cells were incubated for 5 h in X-Vivo 20 at effector:target ratios of 1:1 in 96-well plates, harvested and stained for surface markers, Annexin V FITC (BD Biosciences) and Propidium iodide (Sigma-Aldrich), according to the manufacturer's protocols. CD4⁺ T cells were isolated from PBMC using a CD4 positive isolation kit (Dynal Biotech, Oslo, Norway). Cells within the live scatter gate, and staining negatively for Annexin V and Propidium iodide, were considered live. A fixed number of fluorescent polymer beads was added where indicated to allow calculation of the absolute number of remaining, live target cells in each sample. Each assay was performed in triplicates. The following formula was used to calculate lysis: % specific lysis=1−(live target cells in wells with responders/live targets in control wells).

Semi-Quantitative Reverse Transcriptase Polymerase Chain Reaction (RT PCR)

The cell lines JVM-2 and THP-1 were screened for CD20 mRNA by RT PCR. Total RNA was isolated with miRNAeasy (Qiagen, Hilden, Germany) and cDNA was synthesized from 0.2 μg total RNA with TaqMan Reverse Transcription Reagents (Applied Biosystems, Foster City, Calif., USA) according to the manufacturer's recommendations. PCR was performed with TaqMan PCR core reagents, and primers and probes from Applied Biosystems (CD20 #Hs00544818.ml and GAPD #Hs99999905.ml) on an ABI 7700 machine. The amplification program was 95° C., 10 min, followed by 40 cycles of 95° C. 15 sec-60° C. 90 sec. PCR on all cell lines were performed with three dilutions of cDNA with technical duplicates. CD20 mRNA expression was determined relative to GAPD using standard curves.

Statistics

Methods were as per Example 9 save that a 2-tailed Pearson correlation analysis was also performed.

Results

Dendritic Cells Engineered to Present Peptides from CD20 in the Context of Allogenic HLA-A*0201 Rapidly Induced CD20-Specific, Alto-Restricted CD8⁺ T Cells

Monocyte-derived dendritic cells (moDC) from HLA-A*0201 negative donors were transfected with HLA-A*0201 and loaded with a peptide from the B cell antigen CD20. The engineered cells were co-cultured with autologous T cells. On day 12 of co-culture, T cells reactive with the HLA-A*0201-SEQ ID NO.18 complex were detected as cells binding fluorescence labeled HLA-A*0201/SEQ ID NO.18 (A2/SEQ ID NO.18) pentamers. Positive cells were detected in 5 of 6 donors at a median frequency of 0.19% of CD8⁺ T cells (range 0-0.31%).

The T cells were re-stimulated, and pentamer positive cells were found on day 19 in 6/6 donors at a median frequency 0.57% of CD8⁺ T cells (range 0.12-2.79%) (FIG. 12A). Binding of pentamers was clearly peptide dependent, as the median frequencies of T cells binding a control pentamer incorporating a CMV-derived peptide was 0.04% and always lower than the A2/SEQ ID NO.18 pentamer. The frequency of cells binding the two pentamers was similar and low before the co-culture.

We examined whether HLA-A*0201-SEQ ID NO.18 was antigenic in an autologous setting. MoDCs from two separate HLA-A*0201 positive donors were pulsed with the SEQ ID NO.18 peptide and co-cultured with autologous T cells. The frequencies of pentamer positive cells were similar to those staining with a control pentamer even after weekly re-stimulation for 4-5 weeks. These results were consistent with the thymic deletion of T cells reactive with self-peptides presented by self-MHC.

Expanded A2/SEQ ID NO.18 Pentamer-Positive T Cells Displayed an Effector Memory Phenotype

Cells from five different donors staining positively with the A2/SEQ ID NO.18 pentamer were sorted by flow cytometry to a purity of >98%, and expanded in an antigen-independent manner for 2-6 weeks. The resulting T cell lines remained pentamer positive throughout the expansion (FIG. 13A). Cells expanded a median of 245-fold in 2 weeks (range 153-1004) (FIG. 13B). The absolute counts achieved in 2-3 weeks time were comparable to the number of T cells required for adoptive immunotherapy. To characterize the differentiation status of the cultured cells, three of the T cell lines were phenotyped on d37-d42 of culture. The cells were on average 69% CD27+ (range 65-72%), 16% CD28+ (range 15-18%), 95% CD45RO+ (range 90-99%), 19% CD45RA+ (range 9-30%), 8% CD62L (range 5-10%) and 0% CD57+ (FIG. 13C). Thus, even after 40 days of in vitro culture the majority of cells still expressed the co-stimulatory molecule CD27 and were negative for CD45RA. These features were characteristic of early, rather than late, effector memory cells, (Sallusto, Geginat et al. 2004) which is advantageous in an immunotherapy setting.

Allo-Restricted T Cell Lines were Specifically Activated by Target Cells Expressing SEQ ID NO.18 and HLA-A*0201

Four of the pentamer-positive T cell lines were cultured in the presence of HLA-A*0201 positive EBV-LCL expressing CD20, a large fraction of the population produced interferon gamma and released cytotoxic granules. (FIGS. 14A, B). The median frequencies of responding cells were 46.1% (range 24.9-61.3, n=6) for EBV-LCL donor 1, and 51.3% (range 36.2-59.5, n=4) for EBV-LCL donor 2. The response was inhibited by antibodies to HLA class I. EBV-LCL that were negative for HLA-A*0201 did not induce activation, but became stimulatory after transfection with the alloantigen. (FIGS. 14A, B). (median increase 17.7%, range 14.6-35.1, p=0.02, n=4, and FIG. 14B).

Peptide loading led to further increase in the response. The CD20+ B-prolymphocytic leukemia cell line JVM-2 was also efficiently recognized. A median of 50.0% T cells responded (range 28.5-78.5, n=7, and FIG. 14B).

The myelo-monocytic leukemia cell line THP-1 is positive for HLA-A2 but negative for CD20. This cell line induced some activation of the T cell lines, but loading with the SEQ ID NO.18 peptide led to a large and dose-dependent increase in the frequency of activated T cells, which reached statistical significance at a concentration of 100 nM (FIG. 14B, p=0.016, n=4) and above. In all experiments, there was also a higher response to THP-1 cells pulsed with 10 nM of the SEQ ID NO.18 peptide relative to non-pulsed cells. The effect of peptide loading was inhibited by anti-HLA class I. It should be pointed out that no attempts were made to deplete surface-bound peptides on the THP-1 cells prior to peptide loading, and that the cells were washed before incubation with the T lymphocytes. The concentrations of peptide needed for responses are therefore expected to be higher than those required for cytotoxicity assays of e.g. T2 cells, which have low levels of HLA molecules on the surface unless peptide loaded, and present few endogenous peptides. Specificity was further confirmed in a separate experiment, demonstrating that CD4+ T cells induced activation of only 2.9% SEQ ID NO.18-specific T cells, whereas 58.8% of the cells responded following pulsing with SEQ ID NO.18 peptide (data not shown).

The ability of the 4 peptide-specific, allo-restricted CTLs to kill defined cell types was demonstrated by cytotoxicity assays. A reduction in the absolute numbers of viable target cells that remained after CTL co-culture was determined by flow cytometry. The results were similar to those obtained by measuring T cell activation at the single cell level, and demonstrated that efficient killing required the presence of both HLA-A2 and SEQ ID NO.18 peptide on the target cells (FIG. 15). The effects were observed at E:T ratios of only 2:1.

Allo-Restricted, CD20 Specific CTLs Efficiently Killed Primary Chronic Lymphocytic Leukemia Cells

To investigate the potential utility of the SEQ ID NO.18-specific CTLs in immunotherapy of CLL, we tested their ability to kill cells from peripheral blood of CLL patients (Table 3). FIG. 16A shows the data from 3 separate CTL lines and 4 separate experiments. Collectively, the results demonstrate that CLL cells from 4 HLA-A*0201 positive patients were lysed at levels that were highly significant relative to control CD4+ T cells at an E:T ratio of only 1:1. The effect was not dependent on priming of the CLL cells with CD40L, known to dramatically increase their susceptibility to T cell-mediated lysis. HLA-A*0201 negative CLL cells were not lysed. In fact, supplementing the culture with CTL lines enhanced viability, resulting in negative values for lysis. This is perhaps not surprising since T cells express several membrane-bound and secreted factors that may promote survival of B cells in vitro. Among PBMCs, CD4+ T cells are the most susceptible to perforin/granzyme-mediated lysis. (Jie Liu 2007) However, negligible levels of lysis were detected among sorted CD4⁺ HLA-A*0201⁺ cells from healthy donors (FIG. 16).

TABLE 3
Patient characteristics

| | | | | | % CD5/ |
|---|---|---|---|---|---|
| Patient | Sex/ | | | V_{H}-mutational | CD19+ |
| ID | Age | Binet | Karyotype | status | in PBMC |
| CLL 105 | F/61 | B | del13q14.3 | Biallelic: | 86 |
| | | | | 100% homology; | |
| | | | | V_{H}3-23 | |
| | | | | 94.8% homology; | |
| | | | | V_{H}3-49 | |
| CLL 106 | F/53 | A | ND | 95.2% homology; | 84 |
| | | | | V_{H}3-23 | |
| CLL 112 | F/58 | B | del11q22.3 | 98.1% homology; | 88 |
| | | | | V_{H}3-30 | |
| CLL 113 | M/59 | A | Normal | 94% homology; | 89 |
| | | | | V_{H}4-34 | |
| CLL 114 | M/58 | A | Normal | 88.1% homology; | 96 |
| | | | | V_{H}3-23 | |
| CLL 115 | M/42 | A | Normal | 97.2% homology; | 81 |
| | | | | V_{H}3-23 | |

All patients were untreated except for patient CLL 112 who was treated for 3 days with oral fludarabin and cyclophosphamide 25 days prior to donating blood.

Strikingly, a perfect correlation was observed with regard to the relative efficiency with which CLL cells from the 4 HLA-A*0201 positive donors were lysed among experiments 1-3 (r=1). As the correlation between experiment 4 and the other 3 was also good (r=0.6), the consistency across all experiments was very high (average r=0.8). The CLL donor dependent differences in ability to be lysed by the CTL lines was not, however, explained by corresponding levels of CD20 or HLA-A*0201 on the CD5+CD19+ leukemia cells (FIG. 16B). Furthermore, surface levels of CD80 and CD86 were low on the majority of all CLL cells and did not associate with susceptibility to killing (FIG. 16B).

Discussion

It is well established that host-reactive T cells in allo-grafts contribute to eradication of leukemia after AHSCT and donor-lymphocyte infusions. The major limitation is that the response is unpredictable and not restricted to hematopoietic tissue. Example 10 shows that a cytolytic T cell response can be targeted to the B-lymphoid restricted antigen CD20. Tolerance to self was overcome by presenting a CD20-derived peptide on a foreign HLA molecule. The CTLs that were obtained killed primary B-CLL cells with high efficiencies while sparing non-B hematopoietc cells. The efficient and specific killing of the intended target population indicated that CTLs selectively reacting with defined HLA-peptide complexes on hematopoietic cells may be used to eradicate leukemia without causing GVHD.

It was notable that we were unable to detect any A2/CD20 pentamer positive cells after prolonged co-culture of T cells from healthy donors with autologous DCs pulsed with CD20 peptide.

In this example, it is demonstrated that three allo-restricted CD20-specific CTL lines efficiently killed CLL cells from four different donors at an E:T ratio of only 1:1. To achieve lysis it is common to pre-activate the CLL cells with CD40L. The mechanism is believed to involve upregulation of co-stimulatory molecules such as CD80 and CD86. In this example, killing occurred without pre-activation. Moreover, there was no correlation between expression of CD80 and CD86 and susceptibility to T cell lysis. CTL-mediated killing was reduced by anti-HLA class I, and HLA-A*0201 negative CLL cells from two patients were spared, demonstrating MHC-dependency of the response. The high efficiencies by which CLL cells were killed in the present example probably reflected that DCs are superior APCs in allo-responses, in agreement with the results of Example 9.

Example 10 confirms that dendritic cells can be engineered to obtain CTLs that are allo-reactive, yet highly peptide-specific. The results showing that HLA-A2+ T cells were spared by A2/CD20 specific T cells are particularly encouraging, since B-cells and T cells share a large number of proteins. The dramatic increase observed after peptide loading clearly demonstrates that the large majority of T cell receptors were peptide-dependent. A certain degree of non-specific killing is commonly observed also for autologous T cell responses. For example, Bae et al found a nine-fold increase in proliferative activity of CD20-specific CTLs in the presence of HLA-A2+ cells not expressing CD20 compared to no stimulator cells. In another study, CMV-specific CTL induced 15% lysis of HLA-matched target cells in the absence of specific antigen. (Mous, Leukemia 2006, FIG. 2) Importantly, the responses measured in the present example were polyclonal, and the large majority of cells were not activated by THP1. Thus, specificity is likely to be enhanced even further by T cell cloning.

### Example 11

In this example, the T-cell receptors of T-cells specific for the CD20 peptide of SEQ ID NO. 18 were cloned and sequenced.

Materials and Methods

Cell Lines and Clones

T cell lines were generated as described in Example 10 (Induction of T cell lines). T cell clones were generated according to standard procedures, as described in Yee et al, J. Immunol, 1999, 162:2227. Briefly, CD20/A2 pentamer positive cells from T cell lines were cloned by limiting dilution into Terasaki plates onto irradiated (30Gy) allogeneic PBMC's in Cell-Gro medium with 5% human serum, Phyto Hemagglutinin (1 ug/ml) and IL-2 (100 U/ml).

T cell lines and clones were confirmed CD20/A2 pentamer positive by flow cytometry, as described, before pelleting and mRNA isolation for T cell receptor cloning.

The method used, as described below, was based on the previous work of Rosenberg and co-workers (Rosenberg, W. M., P. A. Moss, and J. I. Bell, Variation in human T cell receptor V beta and J beta repertoire: analysis using anchor polymerase chain reaction. Eur J Immunol, 1992. 22(2): p. 541-9).

RNA Isolation:

Half a million to two millions cells isolated from a single clone or from a cell line were used for RNA preparation using Stratgene RNA kit (Absolutely RNA Miniprep kit, Stratagene, #400800) following manufacturer's instructions: the starting volume was 600 ml of lysis bfr. The elution was done in 50 ml of the kit's elution buffer EB, which was pre-warmed at 65° C. and passed twice through the column. Concentration and purity were checked using Nanodrop 1000 (Thermo scientific).

cDNA Preparation:

The cDNA preparation was performed using 20-mer oligo_dT (Invitrogen, #18418-020) and the SuperScript™ III Reverse Transcriptase (Invitrogen, #18080-044).

The following procedure was used:

A mix was prepared:
1 μl oligo_dT 50 μM
1 μl dNTP (10 mM, Roche, #11969064001)
RNA (1 μg)
Final volume was brought to 14.5 μl with DEPC-H₂O

This mix was incubated for 5′ at 65° C. and directly chilled on ice.

To this mix, the following reagents were added:
4 μl First Strand Bfr (part of Invitrogen, #18080-044)
1 μl DTT (part of Invitrogen, #18080-044)
0.5 μl RNasin® Ribonuclease Inhibitor (Stratagene, #N2511)
1 μl SuperScript™ III Reverse Transcriptase

Final Volume=21 μl

The reaction was incubated at 50° C. for 1 hr

RNasin was inactivated at 60° C. for 15′

RNAseH Treatment:

The template RNA was then degraded by a RNAseH treatment. RNAseH was from New England Biolabs (#M0297S) and the reaction was set as follows:

21 μl cDNA+1 μl RNAseH were incubated at 37° C. for 20′

Glycogen/NaAc/Isoprop Precipitation:

The cDNA was cleaned and concentrated by glycogen precipitation:

A mix containing:
22 μl cDNA
0.5 μl Glycogen (20 μg/μl, Fermentas, #R0561)
2.3 μl NaAc 3M pH 5.6
65 μl Et-OH 100%

cDNA was precipitated for 20′ at −20° C.

This incubation was followed by a centrifugation at 16,000×g for 10′.

The pellet was washed twice with 150 μl EtOH 70% with 5′ centrifugation at 16,000×g.

The pellet was dried and resuspended in 11 μl dH₂O (1 μl being run on agarose gel as a control).

3′ Terminal dC-Tailing

The purified cDNA (10 μl) was poly deoxycytosine (dC, Invitrogen, #10217-016) tailed using Terminal deoxyribonucleotide Transferase (TdT, Roche, #03333566001). Before the reaction was started, the cDNA template was boiled 1′ at 95° C. and chill on ice before addition of other reagents.

The mix was:
10 μl cDNA
4 μl RB 5× (part of Roche, #03333566001)
4 μl CoCl₂ (part of Roche, #03333566001)
1 μl dCTP (10 mM)
1 μl TdT (400 U)

This 20 μl mix was incubated for 15′ at 37° C.

Glycogen/NaAc/Isoprop Precipitation:

The cDNA was cleaned and concentrated a second time as before:

A mix containing:
22 μl cDNA
0.5 μl Glycogen (20 μg/μl, Fermentas, #R0561)
2.3 μl NaAc 3M pH 5.6
65 μl Et-OH 100%

cDNA was precipitated for 20′ at −20° C.

This incubation was followed by a centrifugation at 16,000×g for 10′.

The pellet was washed twice with 150 μl EtOH 70% with 5′ centrifugation at 16,000×g.

The pellet was dried and resuspended in 24 μl dH₂O.

Two-Step Amplification of V Chains.

In order to amplify TcR from the previously prepared dC-tailed cDNA, a semi-nested PCR was performed. The 1^{st} PCR of 25 cycles was expected to generate >600 bp fragments. The 2^{nd} PCR of 20 cycles gave rise to fragments >450 bp.

In order to test the quality of the cDNA, actin was also amplified in the first PCR reaction. Furthermore, a run without cDNA was performed in order to verify that the primers were not contaminated.

Both reactions were run with a reverse primer containing the G (Guanosine) and I (Inosine), a modified nucleoside that improves the amplification of the dC tail. The other primers anneal to a part of the constant domain, for the second PCR, the specific primer is closer to the 5′-end of the coding sequence, but still on the constant domain (see list below, these primers were taken FROM MBV 4250, A practical laboratory exercise, Manual from > Spring term 2007, University of Oslo). All primers were ordered HPLC-purified at Eurofins MWG GmbH.

The PCR reactions were performed using TITANIUM Taq DNA Polymerase (Clontech, #639208) and the solutions sent with the polymerase. The deoxynucleosides triphosphate (dNTP, Roche, #11969064001) were purchased separately.

The primers' codes (J) correspond to our database, these are their sequence:

| | |
|---|---|
| J10 ACTIN_fwd | |
| GCTCCGGCATGTGCAA | (SEQ. ID NO. 203) |
| J11 ACTIN_rev | |
| AGGATCTTCATGAGGTAGT | (SEQ. ID NO. 204) |
| J14 TRAC_rev | |
| AGTCAGATTTGTTGCTCCAGGCC | (SEQ. ID NO. 205) |
| J15 TRBC_rev | |
| TTCACCCACCAGCTCAGCTCC | (SEQ. ID NO. 206) |
| J16 pGI15TOPO | |
| CACCGGGIIGGGIIGGGII | (SEQ. ID NO. 207) |
| J20 TRAC_rev_int | |
| ATACGCGTTCTCTCAGCTGGTACACGG | (SEQ. ID NO. 208) |
| J22 TRBC_rev_int | |
| ATACGCGTAGATCTCTGCTTCTGATGGC | (SEQ. ID NO. 209) |

Procedure:

1^{st} PCR

| Primers: | cDNA | No cDNA |
|---|---|---|
| J16/J14 TCRA | 1 | 2 |
| J16/J15 TCRB | 3 | 4 |
| J10/J11 ACTIN | 5 | 6 |

The PCR Mix:

0.5 μl cDNA

1.5 μl each primer 10 μM

1.5 μl dNTP 10 mM

1 μl Titanium Tag polymerase

5 μl Titanium buffer 10×

38.5 μl H₂O

50 μl

PCR Conditions:

4′ 94° C.

25×(1′ 94° C., 1′ 53° C. and 1′ 68° C.)

7′ 68° C. and 4° C.∞.

5 μl of this PCR is checked on gel

2^{nd} PCR

| | 73/6 | MART-1 | Jurkat |
|---|---|---|---|
| J16/J20 TCRA | 1 | 2 | 3 |
| J16/J22 TCRB | 4 | 5 | 6 |
| J4/J23 (TCRCb, EX13) | 7 | 8 | X |

The PCR Mix:

1/50 of the first PCR (1 μl)

1.5 μl each primer 10 μM

1.5 μl dNTP 10 mM

1 μl Titanium Taq polymerase

5 μl Titanium buffer 10×

38.5 μl H₂O

50 μl

PCR Conditions:

4′ 94° C.

20×(1′ 94° C., 1′ 53° C. and 30″ 68° C.)

7′ 68° C. and 4° C.∞.

Purification of Amplified TCR Fragments

Half (25 μl/50 μl) of the second PCR reaction was run on agarose gel and the DNA was gel-purified using E.Z.N.A® Cycle Pure kit (#D6492-02) following instructions. In the final step, the DNA was eluted with 30 μl of pre-warmed elution buffer and 3 μl was run on agarose gel for estimation of the amount.

Cloning into pGEM-T

The purified PCR fragment was ligated into pGEMT easy cloning vector (Promega, #A1360) following the instructions. The cloned DNA was then used to transform XL1-Blue competent Cells (Stratagene, #200249) following the instructions.

Miniprep and Sequencing

Blue/white screen of colonies was performed. Miniprep of white colonies was done using NucleoBond PC 20 (Macherey-Nagel, #740571) following instructions. The plasmids were resuspended in 100 μl elution buffer and 15 μl was sent for sequencing to Eurofins MWG GmbH.

Results

The CDR3 region as well as the numbering of the different domains is done according to the IMGT database; the first C residue belonging to the Vbeta domain and the last F residue belonging to the J-domain. The amino acids in between are part of the CDR3 region. Since the length of the region can vary from one clone to the other, the numbering of the amino acids is done as follows: +aa1=first amino acid after C (from the Vbeta), +aa2=second amino acid after C, etc. These amino acids can also be designated according to their location from F (of the J-domain): −aa-1=first amino acid before F, etc.

The sequences of the CDR3 regions of the T-cell receptors specific for the peptide of SEQ ID NO. 18 are shown in Tables 4 and 5. Table 4 also shows the CDR3 regions of the β chain of T-cell receptors specific for the MART-1 protein, for comparative purposes.

In Tables 4 and 5, the designation “-” indicates that the adjacent amino acid residues are joined to each other and serves to distinguish the CDR3 regions from adjacent germline-encoded residues (principally an N-terminal cysteine residue and a C-terminal phenylalanine residue) and to assist in visual alignment of consensus sequences.

In Table 5, “Source” refers to the cell material from which the T cell receptors have been cloned. “Clone” refers to CD20-pentamer positive T cell clones, and “Line” refers to a CD20-pentamer positive T cell line. Where T cell clones have been used as the starting material, the corresponding alpha and beta chains listed occur as natural pairs. Where the T cell line has been used, it is not known which alpha chain is paired with which beta chain, as marked by a vertical line between the TRBJ and TRAV.

TABLE 4

| Protein for | | | |
|---|---|---|---|
| which TCR | Sequence of CDR3 | Arrangement of | |
| is specific | region of β chain | Gene Segments | SEQ ID NO. |
| CD20 | C-A [symbol] HGNG [symbol] QY-F | TRBV4-2-J2-7D2 | 148 |
| CD20 | C-A [symbol] WGQKNIQY-F | TRBV13-J2-4D1 | 149 |
| CD20 | C-ATAPGLS [symbol] QY-F | TRBV19-J2-7D2 | 150 |
| CD20 | C-A [symbol] WGRA [symbol] QY-F | TRBV6-5-J2-7D1 | 151 |
| CD20 | C-A [symbol] AGVG [symbol] QY-F | TRBV6-4′02-J2-7D1 | 152 |
| CD20 | C-A [symbol] LGSANTQY-F | TRBV13-J2-3 (no D) | 153 |
| CD20 | C-A [symbol] PGLT [symbol] QY-F | TRBV12-4-J2-7D2 | 154 |
| CD20 | C-A [symbol] QGLT [symbol] QY-F | TRBV4-2-J2-7D1 | 155 |
| CD20 | C-A [symbol] LGNRIGELF-F | TRBV13-J2-2 | 170 |
| CD20 | C-A [symbol] PGLT [symbol] QY-F | TRBV3-1-J2-7 | 171 |
| CD20 | C-A [symbol] VGGG [symbol] QY-F | TRBV9-J2-7 | 172 |
| CD20 | C-A [symbol] IG-- [symbol] QF-F | TRBV19-J2-1 | 173 |
| CD20 | C-A [symbol] LGQT [symbol] QY-F | TRBV7-6-J2-7 | 174 |
| MART-1 | C-AS [symbol] FGGLGQPQH-F | J1 | 156 |
| MART-1 | C-AS [symbol] DSGVGQPQH-F | J1 | 157 |
| MART-1 | C-AS [symbol] PGPLLTEAF-F | J1 | 158 |
| MART-1 | C-AS [symbol] LGTGGRGYT-F | J1 | 159 |
| MART-1 | C-AS [symbol] QDPGLHQPQH-F | J1 | 160 |
| MART-1 | C-AS [symbol] FTGTVNYGYT-F | J1 | 161 |
| MART-1 | C-AS [symbol] LASGVNTEAF-F | J1 | 162 |
| MART-1 | C-AS [symbol] PVGLSGNTIYF | J1 | l63 |
| MART-1 | C-AS [symbol] SPNRQGDYGYT-F | J1 | 164 |
| MART-1 | C-ASISFQRQAPNSPLH-F | J1 | 165 |
| MART-1 | C-AS [symbol] YLGQPTLNSANTGELF-F | J1 | 166 |

[symbol]

indicates data missing or illegible when filed

It is apparent that there are certain conserved amino acid residues in the CDR3 region of the TcR beta chain that forms part of a T-cell receptor that is specific for the peptide of SEQ ID NO. 18. Accordingly, the following motifs for such CDR3 regions have been developed. Firstly, it is notable that the amino acid at position +5 is universally a glycine residue in the T-cell receptors specific for SEQ ID NO. 18. It is also notable that the CDR3 regions of these T-cell receptors are encoded by J-2 gene segments, namely segments J2-7, J2-1, J2-2, J2-3 or J2-4, the peptide sequences of which are shown in Table 6. It is, further notable, that the amino acid at position −1 is universally an aromatic amino acid (tyrosine or phenylalanine). In most of the CDR3 regions of these T-cell receptors, positions −2 and −1 are glutamine and tyrosine, respectively, and in many of these CDR3 regions, position −3 is glutamic acid. The CDR3 regions of these T-cell receptors are between 9 and 12 amino acid residues, with 11 amino acid residues being most common. The following consensus sequences have also been developed.

| | |
|---|---|
| ASSXGXXXXQY | (SEQ ID NO. 167) |
| AXXXGXXYEQY | (SEQ ID NO. 168) |
| ASSXGXXYEQY | (SEQ ID NO. 169) |
| ASSXGXXX(XXX)F | (SEQ ID NO. 189) |

where amino acids in parentheses may be omitted

TABLE 6

| | | SEQ. ID |
|---|---|---|
| J gene segment | Peptide Sequence | NO. |
| TRBJ2-1*01 | SYNEQFFGPGTRLTVL | 191 |
| TRBJ2-2*01 | NTGELFFGEGSRLTVL | 192 |
| TRBJ2-2P*01 | LRGAAGRLGGGLLVL | 193 |
| TRBJ2-3*01 | STDTQYFGPGTRLTVL | 194 |
| TRBJ2-4*01 | AKNIQYFGAGTRLSVL | 195 |
| TRBJ2-5*01 | QETQYFGPGTRLLVL | 196 |
| TRBJ2-6*01 | SGANVLTFGAGSRLTVL | 197 |
| TRBJ2-7*01 | SYEQYFGPGTRLTVT | 198 |

TABLE 5

| | | SEQ ID | | | | SEQ ID | | | |
|---|---|---|---|---|---|---|---|---|---|
| TRBV | CDR3 | NO. | TRBJ | TRAV | CDR3 | NO. | TRAJ | Source | Donor |
| TRBV4-2 | C-ASSHGNGYEQY-F | 148 | J2-7 | TRAV12-2 | C-AGIMDSNY | 175 | J-33 | clone | 1 |
| TRBV13 | C-ASSWGQKNIQY-F | 149 | J2-4 | TRAV10 | C-WSGLGGSQGNLI-F | 176 | J-42 | | 2 |
| TRBV19 | C-ATAPGLSYEQY-F | 150 | J2-7 | TRAV30 | C-GTQGGSEKLV-F | 177 | J-57 | | 2 |
| TRBV6-5 | C-ASSWGRAYEQY-F | 151 | J2-7 | TRAV12-2 | C-AVVNDMR-F | 178 | J-43 | | 2 |
| TRBV6-4 | C-ASSAGVGYEQY-F | 152 | J2-7 | TRAV12-2 | C-AVNPY | 190 | J-33 | | 2 |
| TRBV13 | C-ASSLGSANTQY-F | 153 | J2-3 | TRAV38-2/DV8 | C-AYASFDMR-F | 179 | J-43 | | 2 |
| TRBV12-4 | C-ASSPGLTYEQY-F | 154 | J2-7 | TRAV5 | C-AEWGQGGKLI-F | 180 | J-23 | | 2 |
| TRBV4-2 | C-ASSQGLTYEQY-F | 155 | J2-7 | TRAV24 | C-AFMPGAGSYQLT-F | 181 | J-28 | | 2 |
| TRBV13 | C-ASSLGNRIGELF-F | 170 | J2-2 | TRAV12-2 | C-AVSGGSYIPT-F | 182 | J-6 | Line | 3 |
| TRBV3-1 | C-ASSPGLTYEQY-F | 171 | J2-7 | TRAV12-2 | C-AGTGGSYIPT-F | 183 | J-6 | | 3 |
| TRBV9 | C-ASSVGGGYEQY-F | 172 | J2-7 | TRAV12-2 | C-AVTGNTPLV-F | 184 | J-29 | | 3 |
| TRBV19 | C-ASSIG--YEQF-F | 173 | J2-1 | TRAV21 | C-AALNRDDKII-F | 185 | J-30 | | 3 |
| TRBV7-6 | C-ASSLGQTYEQY-F | 174 | J2-7 | TRAV38-2/DV8 | C-AFPYNTDKLI-F | 186 | J-34 | | 3 |
| | | | | TRAV8-4 | C-AVSEKTGSGNTGKLI-F | 187 | J-37 | | 3 |
| | | | | TRAV9-2 | C-ALGYSGAGSYQLT-F | 188 | J-28 | | 3 |

1-67. (canceled)

68. A method of treating cancer or a disease related to a dysfunction in hematopoietic cells in a patient comprising administering a T-cell receptor, or a peptide-binding fragment thereof to the patient, wherein the T-cell receptor is capable of binding a polypeptide consisting of the sequence of the peptide of SEQ ID NO: 18, or a sequence having at least 60% sequence identity thereto, when the peptide is presented on an HLA molecule of a first HLA allele, and wherein:
i) the T-cell receptor is obtainable from an individual who is negative for said first HLA allele;
ii) at least the complementarity determining regions of the T-cell receptor are the same as those present in a T-cell receptor in accordance with i); or
iii) at least the complementarity determining regions of the T-cell receptor are the same as those present in a T-cell receptor in accordance with i) with one or more amino acid omissions or substitutions in at least one of the complementarity determining regions.

69. A method according to claim 68, wherein the CDR3 region of the beta chain of the T-cell receptor comprises a glycine residue at position 5 from the N-terminus.

70. A method according to claim 68, wherein the C-terminal residues of the CDR3 region of the beta chain of the T-cell receptor are encoded by a J-2 gene segment.

71. A method according to claim 70, wherein at least the two C-terminal residues of the CDR3 region of the beta chain of the T-cell receptor are encoded by a J-2 gene segment.

72. A method according to claim 68, wherein the CDR3 region of the beta chain of the T-cell receptor comprises amino acids encoded by a J-2 gene segment selected from the group consisting of J2-7, J2-5, J2-1, J2-2, J2-3 and J2-4.

73. A method according to claim 68, wherein the C-terminal residue of the CDR3 region of the beta chain of the T-cell receptor is an aromatic amino acid.

74. A method according to claim 73, wherein the aromatic amino acid is selected from the group consisting of tyrosine and phenylalanine.

75. A method according to claim 68, wherein the two amino acid residues at the C-terminus of the CDR3 region of the beta chain of the T-cell receptor consist of the sequence Gln Tyr.

76. A method according to claim 75, wherein the three amino acid residues at the C-terminus of the CDR3 region of the beta chain of the T-cell receptor consist of the sequence Glu Gln Tyr.

77. A method according to claim 68, wherein the CDR3 region of the beta chain of the T-cell receptor consists of between 9 and 12 amino acid residues.

78. A method according to claim 77, wherein the CDR3 region of the beta chain of the T-cell receptor consists of 11 amino acid residues.

79. A method according to claim 68, wherein the CDR3 region of the β chain of the T-cell receptor comprises a sequence selected from the group consisting of SEQ. ID NO. 167, 168 and 189.

80. A method according to claim 68, wherein the CDR3 region of the β chain of the T-cell receptor comprises the sequence of SEQ. ID NO. 169.

81. A method according to claim 68, wherein the CDR3 region of the β chain of the T-cell receptor comprises a sequence selected from the group consisting of any one of SEQ. ID NOS. 148 to 155 or 170 to 174.

82. A method according to claim 68, wherein the CDR3 region of the a chain of the T-cell receptor comprises a sequence selected from the group consisting of any one of SEQ. ID NOS. 175 to 188 and 190.

83. A method according to claim 68, wherein the first HLA allele is an HLA-A2 allele.

84. A method of treating cancer or a disease related to a dysfunction in hematopoietic cells in a patient comprising administering a polynucleotide to the patient, wherein the polynucleotide comprises a sequence encoding a T-cell receptor, or a polypeptide-binding fragment thereof in accordance with claim 68.

85. A method of treating cancer or a disease related to a dysfunction in hematopoietic cells in a patient comprising administering a T-cell displaying at least a T-cell receptor according to claim 68, to the patient, wherein the patient is positive for the first HLA allele.

86. A method according to claim 85, further comprising the simultaneous or sequential administration of a second T-cell which displays at least a second T-cell receptor capable of binding a second peptide, present in the patient, when presented by an HLA molecule of the first HLA allele, the second T-cell receptor:
i) being obtainable from an individual who is negative for the first HLA allele;
ii) having at least the same complementarity determining regions as a T-cell receptor obtainable from an individual who is negative for the first HLA allele; or
iii) having complementarity determining regions the same as those present in a T-cell receptor obtainable from an individual who is negative for the first HLA allele with one or more amino acid omissions or substitutions in at least one of the complementarity determining regions.

87. A kit suitable for treating a patient suffering from cancer or a disease related to a dysfunction in hematopoietic cells, the patient being positive for a first HLA allele, the kit comprising:
a first T-cell receptor according to claim 68; and
(ii) a second T-cell receptor, wherein the second T-cell receptor is capable of binding a peptide consisting of between 7 and 100 amino acids and comprising a sequence selected from the group consisting of any one of SEQ. ID NOS. 1 to 17 and 19 to 145 and a sequence having at least 60% identity thereto.


SEQUENCE LISTING (209 sequences)

SEQ ID NO: 1 - protein, 9 aa, homo sapiens
YLLDGILFI

SEQ ID NO: 2 - protein, 9 aa, homo sapiens
YLKDGWHMV

SEQ ID NO: 3 - protein, 9 aa, homo sapiens
ILLWEIFSL

SEQ ID NO: 4 - protein, 9 aa, homo sapiens
FLYIGIITI

SEQ ID NO: 5 - protein, 9 aa, homo sapiens
YLAVGIMFL

SEQ ID NO: 6 - protein, 9 aa, homo sapiens
SLFEGIYTI

SEQ ID NO: 7 - protein, 9 aa, homo sapiens
FLDCQIHRV

SEQ ID NO: 8 - protein, 9 aa, homo sapiens
FLLGLGLGV

SEQ ID NO: 9 - protein, 9 aa, homo sapiens
GMDRYLIYV

SEQ ID NO: 10 - protein, 9 aa, homo sapiens
PLWGGIMYI

SEQ ID NO: 11 - protein, 9 aa, homo sapiens
SLQSGILTL

SEQ ID NO: 12 - protein, 9 aa, homo sapiens
FLVNTVLWV

SEQ ID NO: 13 - protein, 9 aa, homo sapiens
LLDPKLCYL

SEQ ID NO: 14 - protein, 9 aa, homo sapiens
YLLRVIETL

SEQ ID NO: 15 - protein, 9 aa, homo sapiens
SLYGPGSYL

SEQ ID NO: 16 - protein, 9 aa, homo sapiens
FLPDAFVTM

SEQ ID NO: 17 - protein, 9 aa, homo sapiens
VLDIYGFEV

SEQ ID NO: 18 - protein, 9 aa, homo sapiens
SLFLGILSV

SEQ ID NO: 19 - protein, 9 aa, homo sapiens
SLAQVVMKV

SEQ ID NO: 20 - protein, 9 aa, homo sapiens
YLMDLSYSM

SEQ ID NO: 21 - protein, 9 aa, homo sapiens
SLWRRPFNV

SEQ ID NO: 22 - protein, 9 aa, homo sapiens
RLYENRASL

SEQ ID NO: 23 - protein, 9 aa, homo sapiens
SMLDDLRNV

SEQ ID NO: 24 - protein, 9 aa, homo sapiens
ILFAFVSSV

SEQ ID NO: 25 - protein, 9 aa, homo sapiens
TLQPPWVSV

SEQ ID NO: 26 - protein, 9 aa, homo sapiens
LLMSFFILL

SEQ ID NO: 27 - protein, 9 aa, homo sapiens
LLLPLLLAL

SEQ ID NO: 28 - protein, 9 aa, homo sapiens
LLSGFFFFA

SEQ ID NO: 29 - protein, 9 aa, homo sapiens
ALAGVLAIL

SEQ ID NO: 30 - protein, 9 aa, homo sapiens
KLFTSVFGV

SEQ ID NO: 31 - protein, 9 aa, homo sapiens
SLHSETWEI

SEQ ID NO: 32 - protein, 9 aa, homo sapiens
GLLSPQAQV

SEQ ID NO: 33 - protein, 9 aa, homo sapiens
VLQELNVTV

SEQ ID NO: 34 - protein, 9 aa, homo sapiens
VLIGILLLV

SEQ ID NO: 35 - protein, 9 aa, homo sapiens
LLLQVSSRV

SEQ ID NO: 36 - protein, 9 aa, homo sapiens
DMKDSIWWV

SEQ ID NO: 37 - protein, 9 aa, homo sapiens
HLYGSNASL

SEQ ID NO: 38 - protein, 9 aa, homo sapiens
LLRTGGWKV

SEQ ID NO: 39 - protein, 9 aa, homo sapiens
LMIPAGIYA

SEQ ID NO: 40 - protein, 9 aa, homo sapiens
GLYQGLSTA

SEQ ID NO: 41 - protein, 9 aa, homo sapiens
LLIHNNFGV

SEQ ID NO: 42 - protein, 9 aa, homo sapiens
RLFEQVMRI

SEQ ID NO: 43 - protein, 9 aa, homo sapiens
VLTPAQLNV

SEQ ID NO: 44 - protein, 9 aa, homo sapiens
GMGWAAFNL

SEQ ID NO: 45 - protein, 9 aa, homo sapiens
KMAEAYSEI

SEQ ID NO: 46 - protein, 9 aa, homo sapiens
LLAEVTLAI

SEQ ID NO: 47 - protein, 9 aa, homo sapiens
ILYPINILI

SEQ ID NO: 48 - protein, 9 aa, homo sapiens
VLFHNLPSL

SEQ ID NO: 49 - protein, 9 aa, homo sapiens
YLIFCLCSL

SEQ ID NO: 50 - protein, 9 aa, homo sapiens
LLLSGAARA

SEQ ID NO: 51 - protein, 9 aa, homo sapiens
YLRPGQAAA

SEQ ID NO: 52 - protein, 9 aa, homo sapiens
ALARGLPGA

SEQ ID NO: 53 - protein, 9 aa, homo sapiens
EMDEELHYA

SEQ ID NO: 54 - protein, 9 aa, homo sapiens
KLMSPKLYV

SEQ ID NO: 55 - protein, 9 aa, homo sapiens
SMSEEVTYA

SEQ ID NO: 56 - protein, 9 aa, homo sapiens
NLEEDAYWV

SEQ ID NO: 57 - protein, 9 aa, homo sapiens
LLLLLIFIV

SEQ ID NO: 58 - protein, 9 aa, homo sapiens
SICEGTVEV

SEQ ID NO: 59 - protein, 9 aa, homo sapiens
FIYGVILTA

SEQ ID NO: 60 - protein, 9 aa, homo sapiens
ILTALFLRV

SEQ ID NO: 61 - protein, 9 aa, homo sapiens
FMQPLFLVA

SEQ ID NO: 62 - protein, 9 aa, homo sapiens
WLQAQKVQV

SEQ ID NO: 63 - protein, 9 aa, homo sapiens
GLADVAVGA

SEQ ID NO: 64 - protein, 9 aa, homo sapiens
IMSDSNYVV

SEQ ID NO: 65 - protein, 9 aa, homo sapiens
RLYQFLLDL

SEQ ID NO: 66 - protein, 9 aa, homo sapiens
KLLLALAGV

SEQ ID NO: 67 - protein, 9 aa, homo sapiens
LLIILLAEV

SEQ ID NO: 68 - protein, 9 aa, homo sapiens
YISRDTCIV

SEQ ID NO: 69 - protein, 9 aa, homo sapiens
FLQDTFPQQ

SEQ ID NO: 70 - protein, 9 aa, homo sapiens
SMEEAKQLV

SEQ ID NO: 71 - protein, 9 aa, homo sapiens
TLPQLFLKV

SEQ ID NO: 72 - protein, 9 aa, homo sapiens
ALASVLLAL

SEQ ID NO: 73 - protein, 9 aa, homo sapiens
ELLDVSWLI

SEQ ID NO: 74 - protein, 9 aa, homo sapiens
LLEGNLVTL

SEQ ID NO: 75 - protein, 9 aa, homo sapiens
GLMATPAKL

SEQ ID NO: 76 - protein, 9 aa, homo sapiens
AIWLFIFNV

SEQ ID NO: 77 - protein, 9 aa, homo sapiens
LLLTISLLV

SEQ ID NO: 78 - protein, 9 aa, homo sapiens
SVDPRIANV

SEQ ID NO: 79 - protein, 9 aa, homo sapiens
LMGTLIAKV

SEQ ID NO: 80 - protein, 9 aa, homo sapiens
LIQPFMFRL

SEQ ID NO: 81 - protein, 9 aa, homo sapiens
LVAPRFLEV

SEQ ID NO: 82 - protein, 9 aa, homo sapiens
LLWAGALAM

SEQ ID NO: 83 - protein, 9 aa, homo sapiens
FLLLTISLL

SEQ ID NO: 84 - protein, 9 aa, homo sapiens
VLLRWEEEL

SEQ ID NO: 85 - protein, 9 aa, homo sapiens
VLLVVLLVV

SEQ ID NO: 86 - protein, 9 aa, homo sapiens
YLALGDQML

SEQ ID NO: 87 - protein, 9 aa, homo sapiens
LLLTNTFGA

SEQ ID NO: 88 - protein, 9 aa, homo sapiens
YLGYTVTWL

SEQ ID NO: 89 - protein, 9 aa, homo sapiens
HLLGPWLLL

SEQ ID NO: 90 - protein, 9 aa, homo sapiens
RMPWYHSSL

SEQ ID NO: 91 - protein, 9 aa, homo sapiens
KMIMNSLSL

SEQ ID NO: 92 - protein, 9 aa, homo sapiens
TLWQLVEYL

SEQ ID NO: 93 - protein, 9 aa, homo sapiens
KLKDLFTEL

SEQ ID NO: 94 - protein, 9 aa, homo sapiens
GMVFSLLQV

SEQ ID NO: 95 - protein, 9 aa, homo sapiens
VLWPRACWT

SEQ ID NO: 96 - protein, 9 aa, homo sapiens
NVAELLHQV

SEQ ID NO: 97 - protein, 9 aa, homo sapiens
WLSAAPTSL

SEQ ID NO: 98 - protein, 9 aa, homo sapiens
ELSEDSSNV

SEQ ID NO: 99 - protein, 9 aa, homo sapiens
LMSFFILLL

SEQ ID NO: 100 - protein, 9 aa, homo sapiens
VLWHWLLRT

SEQ ID NO: 101 - protein, 9 aa, homo sapiens
HMRPLAIWL

SEQ ID NO: 102 - protein, 9 aa, homo sapiens
YLHVALDLL

SEQ ID NO: 103 - protein, 9 aa, homo sapiens
LLFLTPMEV

SEQ ID NO: 104 - protein, 9 aa, homo sapiens
KIVEWDWKI

SEQ ID NO: 105 - protein, 9 aa, homo sapiens
VIVGSIIMV

SEQ ID NO: 106 - protein, 9 aa, homo sapiens
FVANAIIHL

SEQ ID NO: 107 - protein, 9 aa, homo sapiens
RLLFFLLFL

SEQ ID NO: 108 - protein, 9 aa, homo sapiens
LLLREHNRL

SEQ ID NO: 109 - protein, 9 aa, homo sapiens
ALAVISFLL

SEQ ID NO: 110 - protein, 9 aa, homo sapiens
ALYDKTKRI

SEQ ID NO: 111 - protein, 9 aa, homo sapiens
WMGGVSEPL

SEQ ID NO: 112 - protein, 9 aa, homo sapiens
CMAGARVQV

SEQ ID NO: 113 - protein, 9 aa, homo sapiens
KLSLGLPGL

SEQ ID NO: 114 - protein, 9 aa, homo sapiens
LLFIVVLTL

SEQ ID NO: 115 - protein, 9 aa, homo sapiens
VMNGTDPEL

SEQ ID NO: 116 - protein, 9 aa, homo sapiens
LLMVFVALL

SEQ ID NO: 117 - protein, 9 aa, homo sapiens
SLTLGNVFV

SEQ ID NO: 118 - protein, 9 aa, homo sapiens
TVQEGLCVL

SEQ ID NO: 119 - protein, 9 aa, homo sapiens
SLFAAISGM

SEQ ID NO: 120 - protein, 9 aa, homo sapiens
VLAEASASQ

SEQ ID NO: 121 - protein, 9 aa, homo sapiens
LLQRPGLQL

SEQ ID NO: 122 - protein, 9 aa, homo sapiens
ALPAALAVI

SEQ ID NO: 123 - protein, 9 aa, homo sapiens
SMFHVTLKI

SEQ ID NO: 124 - protein, 9 aa, homo sapiens
KLGGDLLRA

SEQ ID NO: 125 - protein, 9 aa, homo sapiens
ILLLVIWKA

SEQ ID NO: 126 - protein, 9 aa, homo sapiens
FLYGVILTV

SEQ ID NO: 127 - protein, 9 aa, homo sapiens
FLQDTFPQV

SEQ ID NO: 128 - protein, 9 aa, homo sapiens
FLPDAFVTV

SEQ ID NO: 129 - protein, 9 aa, homo sapiens
YLMDLSYSV

SEQ ID NO: 130 - protein, 9 aa, homo sapiens
LLQPFMFRV

SEQ ID NO: 131 - protein, 9 aa, homo sapiens
LLIPAGIYV

SEQ ID NO: 132 - protein, 9 aa, homo sapiens
ELDEELHYV

SEQ ID NO: 133 - protein, 9 aa, homo sapiens
LLLSGAARV

SEQ ID NO: 134 - protein, 9 aa, homo sapiens
SLDPRIANV

SEQ ID NO: 135 - protein, 9 aa, homo sapiens
GLYQGLSTV

SEQ ID NO: 136 - protein, 9 aa, homo sapiens
FLQPLFLVV

SEQ ID NO: 137 - protein, 9 aa, homo sapiens
LLAPRFLEV

SEQ ID NO: 138 - protein, 9 aa, homo sapiens
ALARGLPGV

SEQ ID NO: 139 - protein, 9 aa, homo sapiens
YLRPGQAAV

SEQ ID NO: 140 - protein, 9 aa, homo sapiens
SLSEEVTYV

SEQ ID NO: 141 - protein, 9 aa, homo sapiens
GLADVAVGV

SEQ ID NO: 142 - protein, 9 aa, homo sapiens
LLAEVTLAV

SEQ ID NO: 143 - protein, 9 aa, homo sapiens
LLWAGALAV

SEQ ID NO: 144 - protein, 9 aa, homo sapiens
ILYPINILV

SEQ ID NO: 145 - protein, 9 aa, homo sapiens
ELLDVSWLV

SEQ ID NO: 146 - protein, 9 aa, homo sapiens
GILGFVFTL

SEQ ID NO: 147 - protein, 9 aa, Artificial
  Control Sequence
NLVPMVATV

SEQ ID NO: 148 - protein, 11 aa, Homo sapiens
ASSHGNGYEQY

SEQ ID NO: 149 - protein, 11 aa, Homo sapiens
ASSWGQKNIQY

SEQ ID NO: 150 - protein, 11 aa, Homo sapiens
ATAPGLSYEQY

SEQ ID NO: 151 - protein, 11 aa, Homo sapiens
ASSWGRAYEQY

SEQ ID NO: 152 - protein, 11 aa, Homo sapiens
ASSAGVGYEQY

SEQ ID NO: 153 - protein, 11 aa, Homo sapiens
ASSLGSANTQY

SEQ ID NO: 154 - protein, 11 aa, Homo sapiens
ASSPGLTYEQY

SEQ ID NO: 155 - protein, 11 aa, Homo sapiens
ASSQGLTYEQY

SEQ ID NO: 156 - protein, 12 aa, Homo sapiens
ASSFGGLGQPQH

SEQ ID NO: 157 - protein, 12 aa, Homo sapiens
ASSDSGVGQPQH

SEQ ID NO: 158 - protein, 12 aa, Homo sapiens
ASSPGPLLTEAF

SEQ ID NO: 159 - protein, 12 aa, Homo sapiens
ASSLGTGGRGYT

SEQ ID NO: 160 - protein, 13 aa, Homo sapiens
ASSQDPGLHQPQH

SEQ ID NO: 161 - protein, 13 aa, Homo sapiens
ASSFTGTVNYGYT

SEQ ID NO: 162 - protein, 13 aa, Homo sapiens
ASSLASGVNTEAF

SEQ ID NO: 163 - protein, 13 aa, Homo sapiens
ASSPVGLSGNTIY

SEQ ID NO: 164 - protein, 14 aa, Homo sapiens
ASSSPNRQGDYGYT

SEQ ID NO: 165 - protein, 15 aa, Homo sapiens
ASISFQRQAPNSPLH

SEQ ID NO: 166 - protein, 19 aa, Homo sapiens
ASSYLGQPTLNSANTGELF

SEQ ID NO: 167 - protein, 11 aa, Artificial
  Consensus sequence of CDR3 region of T-cell receptor beta chain
ASSXGXXXXQY

SEQ ID NO: 168 - protein, 11 aa, Artificial
  Consensus sequence of CDR3 region of beta chain of T-cell receptor
AXXXGXXYEQY

SEQ ID NO: 169 - protein, 11 aa, Artificial
  Consensus sequence of CDR3 region of beta chain of T-cell receptor
ASSXGXXYEQY

SEQ ID NO: 170 - protein, 12 aa, Homo sapiens
ASSLGNRIGELF

SEQ ID NO: 171 - protein, 11 aa, Homo sapiens
ASSPGLTYEQY

SEQ ID NO: 172 - protein, 11 aa, Homo sapiens
ASSVGGGYEQY

SEQ ID NO: 173 - protein, 9 aa, Homo sapiens
ASSIGYEQF

SEQ ID NO: 174 - protein, 11 aa, Homo sapiens
ASSLGQTYEQY

SEQ ID NO: 175 - protein, 8 aa, Homo sapiens
AGIMDSNY

SEQ ID NO: 176 - protein, 12 aa, Homo sapiens
WSGLGGSQGNLI

SEQ ID NO: 177 - protein, 10 aa, Homo sapiens
GTQGGSEKLV

SEQ ID NO: 178 - protein, 7 aa, Homo sapiens
AVVNDMR

SEQ ID NO: 179 - protein, 8 aa, Homo sapiens
AYASFDMR

SEQ ID NO: 180 - protein, 10 aa, Homo sapiens
AEWGQGGKLI

SEQ ID NO: 181 - protein, 12 aa, Homo sapiens
AFMPGAGSYQLT

SEQ ID NO: 182 - protein, 10 aa, Homo sapiens
AVSGGSYIPT

SEQ ID NO: 183 - protein, 10 aa, Homo sapiens
AVSGGSYIPT

SEQ ID NO: 184 - protein, 9 aa, Homo sapiens
AVTGNTPLV

SEQ ID NO: 185 - protein, 10 aa, Homo sapiens
AALNRDDKII

SEQ ID NO: 186 - protein, 10 aa, Homo sapiens
AFPYNTDKLI

SEQ ID NO: 187 - protein, 15 aa, Homo sapiens
AVSEKTGSGNTGKLI

SEQ ID NO: 188 - protein, 13 aa, Homo sapiens
ALGYSGAGSYQLT

SEQ ID NO: 189 - protein, 12 aa, Artificial
  CDR3 beta chain consensus sequence
ASSXGXXXXXXF

SEQ ID NO: 190 - protein, 5 aa, Homo sapiens
AVNPY

SEQ ID NO: 191 - protein, 16 aa, Homo sapiens
SYNEQFFGPGTRLTVL

SEQ ID NO: 192 - protein, 16 aa, Homo sapiens
NTGELFFGEGSRLTVL

SEQ ID NO: 193 - protein, 15 aa, Homo sapiens
LRGAAGRLGGGLLVL

SEQ ID NO: 194 - protein, 16 aa, Homo sapiens
STDTQYFGPGTRLTVL

SEQ ID NO: 195 - protein, 16 aa, Homo sapiens
AKNIQYFGAGTRLSVL

SEQ ID NO: 196 - protein, 15 aa, Homo sapiens
QETQYFGPGTRLLVL

SEQ ID NO: 197 - protein, 17 aa, Homo sapiens
SGANVLTFGAGSRLTVL

SEQ ID NO: 198 - protein, 15 aa, Homo sapiens
SYEQYFGPGTRLTVT

SEQ ID NO: 199 - protein, 270 aa, Homo sapiens
MMKSLGVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVVNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 200 - protein, 357 aa, Homo sapiens [sequence omitted]

SEQ ID NO: 201 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 202 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 203 - DNA, 16 bp, Artificial
  Primer sequence
gctccggcatgtgcaa

SEQ ID NO: 204 - DNA, 19 bp, Artificial
  Primer sequence
aggatcttcatgaggtagt

SEQ ID NO: 205 - DNA, 23 bp, Artificial
  Primer sequence
agtcagatttgttgctccaggcc

SEQ ID NO: 206 - DNA, 21 bp, Artificial
  Primer sequence
ttcacccaccagctcagctcc

SEQ ID NO: 207 - DNA, 19 bp, Artificial
  Primer sequence
caccgggnngggnngggnn

SEQ ID NO: 208 - DNA, 27 bp, Artificial
  Primer sequence
atacgcgttctctcagctggtacacgg

SEQ ID NO: 209 - DNA, 28 bp, Artificial
  Primer sequence
atacgcgtagatctctgcttctgatggc